SEQUENCE LISTING (116 sequences)

SEQ ID NO: 1 - protein, 234 aa, Homo sapiens
MRAPLCLLLLVAHAVDMLALNRRKKQVGTGLGGNCTGCIICSEENGCSTCQQRLFLFIRREGIRQYGKCLHDCPPGYFGIRGQEVNRCKKCGATCESCFSQDFCIRCKRQFYLYKGKCLPTCPPGTLAHQNTRECQGECELGPWGGWSPCTHNGKTCGSAWGLESRVREAGRAGHEEAATCQVLSESRKCPIQRPCPGERSPGQKKGRKDRRPRKDRKLDRRLDVRPRQPGLQP

SEQ ID NO: 2 - DNA, 714 bp, Homo sapiens
gctgcccagatgcgggcgccactctgcctgctcctgctcgtcgcccacgccgtggacatgctcgccctgaaccgaaggaagaagcaagtgggcactggcctggggggcaactgcacaggctgtatcatctgctcagaggagaacggctgttccacctgccagcagaggctcttcctgttcatccgccgggaaggcatccgccagtacggcaagtgcctgcacgactgtccccctgggtacttcggcatccgcggccaggaggtcaacaggtgcaaaaagtgtggggccacttgtgagagctgcttcagccaggacttctgcatccggtgcaagaggcagttttacttgtacaaggggaagtgtctgcccacctgcccgccgggcactttggcccaccagaacacacgggagtgccagggggagtgtgaactgggtccctggggcggctggagcccctgcacacacaatggaaagacctgcggctcggcttggggcctggagagccgggtacgagaggctggccgggctgggcatgaggaggcagccacctgccaggtgctttctgagtcaaggaaatgtcccatccagaggccctgcccaggagagaggagccccggccagaagaagggcaggaaggaccggcgcccacgcaaggacaggaagctggaccgcaggctggacgtgaggccgcgccagcccggcctgcagccctga

SEQ ID NO: 3 - protein, 234 aa, Homo sapiens
MRAPLCLLLLVAHAVDMLALNRRKKQVGTGLGGNCTGCIICSEENGCSTCQQRLFLFIRREGIRRYGKCLHDCPPGYFGIRGQEVNRCKKCGATCESCFSQDFCIRCKRQFYLYKGKCLPTCPPGTLAHQNTRECQGECELGPWGGWSPCTHNGKTCGSAWGLESRVREAGRAGHEEAATCQVLSESRKCPIQRPCPGERSPGQKKGRKDRRPRKDRKLDRRLDVRPRQPGLQP

SEQ ID NO: 4 - protein, 234 aa, Homo sapiens
MRAPLCLLLLVAHAVDMLALNRRKKQVGTGLGGNCTGCIICSEENGCSTCQQRLFLFIRREGIRQYGKCLHDCPPGYFGIRGQEVNRCKKCGATFESCFSQDFCIRCKRQFYLYKGKCLPTCPPGTLAHQNTRECQGECELGPWGGWSPCTHNGKTCGSAWGLESRVREAGRAGHEEAATCQVLSESRKCPIQRPCPGERSPGQKKGRKDRRPRKDRKLDRRLDVRPRQPGLQP

SEQ ID NO: 5 - protein, 234 aa, Homo sapiens
MRAPLCLLLLVAHAVDMLALNRRKKQVGTGLGGNCTGCIICSEENGCSTCQQRLFLFIRREGIRQYGKCLHDCPPGYFGIRGQEVNRCKKCGATCESCFSQDFCIRRKRQFYLYKGKCLPTCPPGTLAHQNTRECQGECELGPWGGWSPCTHNGKTCGSAWGLESRVREAGRAGHEEAATCQVLSESRKCPIQRPCPGERSPGQKKGRKDRRPRKDRKLDRRLDVRPRQPGLQP

SEQ ID NO: 6 - protein, 234 aa, Homo sapiens
MRAPLCLLLLVAHAVDMLALNRRKKQVGTGLGGNCTGCIICSEENGCSTCQQRLFLFIRREGIRQYGKCLHDCPPGYFGIRGQEVNRCKKCGATCESCFSQDFCIRCKRQFYLYKGKYLPTCPPGTLAHQNTRECQGECELGPWGGWSPCTHNGKTCGSAWGLESRVREAGRAGHEEAATCQVLSESRKCPIQRPCPGERSPGQKKGRKDRRPRKDRKLDRRLDVRPRQPGLQP

SEQ ID NO: 7 - DNA, 705 bp, Homo sapiens
atgcgggcgccactctgcctgctcctgctcgtcgcccacgccgtggacatgctcgccctgaaccgaaggaagaagcaagtgggcactggcctggggggcaactgcacaggctgtatcatctgctcagaggagaacggctgttccacctgccagcagaggctcttcctgttcatccgccgggaaggcatccgccggtacggcaagtgcctgcacgactgtccccctgggtacttcggcatccgcggccaggaggtcaacaggtgcaaaaagtgtggggccacttgtgagagctgcttcagccaggacttctgcatccggtgcaagaggcagttttacttgtacaaggggaagtgtctgcccacctgcccgccgggcactttggcccaccagaacacacgggagtgccagggggagtgtgaactgggtccctggggcggctggagcccctgcacacacaatggaaagacctgcggctcggcttggggcctggagagccgggtacgagaggctggccgggctgggcatgaggaggcagccacctgccaggtgctttctgagtcaaggaaatgtcccatccagaggccctgcccaggagagaggagccccggccagaagaagggcaggaaggaccggcgcccacgcaaggacaggaagctggaccgcaggctggacgtgaggccgcgccagcccggcctgcagccctga

SEQ ID NO: 8 - DNA, 705 bp, Homo sapiens
atgcgggcgccactctgcctgctcctgctcgtcgcccacgccgtggacatgctcgccctgaaccgaaggaagaagcaagtgggcactggcctggggggcaactgcacaggctgtatcatctgctcagaggagaacggctgttccacctgccagcagaggctcttcctgttcatccgccgggaaggcatccgccagtacggcaagtgcctgcacgactgtccccctgggtacttcggcatccgcggccaggaggtcaacaggtgcaaaaagtgtggggccacttttgagagctgcttcagccaggacttctgcatccggtgcaagaggcagttttacttgtacaaggggaagtgtctgcccacctgcccgccgggcactttggcccaccagaacacacgggagtgccagggggagtgtgaactgggtccctggggcggctggagcccctgcacacacaatggaaagacctgcggctcggcttggggcctggagagccgggtacgagaggctggccgggctgggcatgaggaggcagccacctgccaggtgctttctgagtcaaggaaatgtcccatccagaggccctgcccaggagagaggagccccggccagaagaagggcaggaaggaccggcgcccacgcaaggacaggaagctggaccgcaggctggacgtgaggccgcgccagcccggcctgcagccctga

SEQ ID NO: 9 - DNA, 705 bp, Homo sapiens
atgcgggcgccactctgcctgctcctgctcgtcgcccacgccgtggacatgctcgccctgaaccgaaggaagaagcaagtgggcactggcctggggggcaactgcacaggctgtatcatctgctcagaggagaacggctgttccacctgccagcagaggctcttcctgttcatccgccgggaaggcatccgccagtacggcaagtgcctgcacgactgtccccctgggtacttcggcatccgcggccaggaggtcaacaggtgcaaaaagtgtggggccacttgtgagagctgcttcagccaggacttctgcatccggcgcaagaggcagttttacttgtacaaggggaagtgtctgcccacctgcccgccgggcactttggcccaccagaacacacgggagtgccagggggagtgtgaactgggtccctggggcggctggagcccctgcacacacaatggaaagacctgcggctcggcttggggcctggagagccgggtacgagaggctggccgggctgggcatgaggaggcagccacctgccaggtgctttctgagtcaaggaaatgtcccatccagaggccctgcccaggagagaggagccccggccagaagaagggcaggaaggaccggcgcccacgcaaggacaggaagctggaccgcaggctggacgtgaggccgcgccagcccggcctgcagccctga

SEQ ID NO: 10 - DNA, 705 bp, Homo sapiens
atgcgggcgccactctgcctgctcctgctcgtcgcccacgccgtggacatgctcgccctgaaccgaaggaagaagcaagtgggcactggcctggggggcaactgcacaggctgtatcatctgctcagaggagaacggctgttccacctgccagcagaggctcttcctgttcatccgccgggaaggcatccgccagtacggcaagtgcctgcacgactgtccccctgggtacttcggcatccgcggccaggaggtcaacaggtgcaaaaagtgtggggccacttgtgagagctgcttcagccaggacttctgcatccggtgcaagaggcagttttacttgtacaaggggaagtatctgcccacctgcccgccgggcactttggcccaccagaacacacgggagtgccagggggagtgtgaactgggtccctggggcggctggagcccctgcacacacaatggaaagacctgcggctcggcttggggcctggagagccgggtacgagaggctggccgggctgggcatgaggaggcagccacctgccaggtgctttctgagtcaaggaaatgtcccatccagaggccctgcccaggagagaggagccccggccagaagaagggcaggaaggaccggcgcccacgcaaggacaggaagctggaccgcaggctggacgtgaggccgcgccagcccggcctgcagccctga

SEQ ID NO: 11 - DNA, 688 bp, Homo sapiens
cctgctcctgctcgtcgcccacgccgtggacatgctcgccctgaaccgaaggaagaagcaagtgggcactggcctggggggcaactgcacaggctgtatcatctgctcagaggagaacggctgttccacctgccagcagaggctcttcctgttcatccgccgggaaggcatccgccagtacggcaagtgcctgcacgactgtccccctgggtacttcggcatccgcggccaggaggtcaacaggtgcaaaaagtgtggggccacttgtgagagctgcttcagccaggacttctgcatccggtgcaagaggcagttttacttgtacaaggggaagtgtctgcccacctgcccgccgggcactttggcccaccagaacacacgggagtgccagggggagtgtgaactgggtccctggggcggctggagcccctgcacacacaatggaaagacctgcggctcggcttggggcctggagagccgggtacgagaggctggccgggctgggcatgaggaggcagccacctgccaggtgctttctgagtcaaggaaatgtcccatccagaggccctgcccaggagagaggagccccggccagaagaagggcaggaaggaccggcgcccacgcaaggacaggaagctggaccgcaggctggacgtgaggccgcgccagcccggcctgcagccctga

SEQ ID NO: 12 - DNA, 657 bp, Homo sapiens
atgcgggcgccactctgcctgctcctgctcgtcgcccacgccgtggacatgctcgccctgaaccgaaggaagaagcaagtgggcactggcctggctgtatcatctgctcagaggagaacggctgttccacctgccagcagaggctcttcctgttcatccgccgggaaggcatccgccagtacggcaagtgcctgcacgactgtccccctgggtacttcggcatccgcggccaggaggtcaacaggtgcaaaaagtgtggggccacttgtgagagctgcttcagccaggacttctgcatccggtgcaagaggcagttttacttgtacaaggggaagtgtctgcccacctgcccgccgggcactttggcccaccagaacacacgggagtgccagggggagtgtgaactgggtccctggggcggctggagcccctgcacacacaatggaaagacctgcggctcggcttggggcctggagagccgggtacgagaggctggccgggctgggcatgaggaggcagccacctgccaggtgctttctgagtcaaggaaatgtcccatccagaggccctgcccaggagagaggagccccggccagaagaagggcaggaaggaccggcgcccacgcaaggacaggaagctggaccgcaggctggacg

SEQ ID NO: 13 - protein, 219 aa, Homo sapiens
MRAPLCLLLLVAHAVDMLALNRRKKQVGTGLAVSSAQRRTAVPPASRGSSCSSAGKASASTASACTTVPLGTSASAARRSTGAKSVGPLVRAASARTSASGARGSFTCTRGSVCPPARRALWPTRTHGSARGSVNWVPGAAGAPAHTMERPAARLGAWRAGYERLAGLGMRRQPPARCFLSQGNVPSRGPAQERGAPARRRAGRTGAHARTGSWTAGWT

SEQ ID NO: 14 - protein, 234 aa, Homo sapiens
IRAPLCLLLLVAHAVDMLALNRRKKQVGTGLGGNCTGCIICSEENGCSTCQQRLFLFIRREGIRQYGKCLHDCPPGYFGIRGQEVNRCKKCGATCESCFSQDFCIRCKRQFYLYKGKCLPTCPPGTLAHQNTRECQGECELGPWGGWSPCTHNGKTCGSAWGLESRVREAGRAGHEEAATCQVLSESRKCPIQRPCPGERSPGQKKGRKDRRPRKDRKLDRRLDVRPRQPGLQP

SEQ ID NO: 15 - DNA, 705 bp, Homo sapiens
atacgggcgccactctgcctgctcctgctcgtcgcccacgccgtggacatgctcgccctgaaccgaaggaagaagcaagtgggcactggcctggggggcaactgcacaggctgtatcatctgctcagaggagaacggctgttccacctgccagcagaggctcttcctgttcatccgccgggaaggcatccgccagtacggcaagtgcctgcacgactgtccccctgggtacttcggcatccgcggccaggaggtcaacaggtgcaaaaagtgtggggccacttgtgagagctgcttcagccaggacttctgcatccggtgcaagaggcagttttacttgtacaaggggaagtgtctgcccacctgcccgccgggcactttggcccaccagaacacacgggagtgccagggggagtgtgaactgggtccctggggcggctggagcccctgcacacacaatggaaagacctgcggctcggcttggggcctggagagccgggtacgagaggctggccgggctgggcatgaggaggcagccacctgccaggtgctttctgagtcaaggaaatgtcccatccagaggccctgcccaggagagaggagccccggccagaagaagggcaggaaggaccggcgcccacgcaaggacaggaagctggaccgcaggctggacgtgaggccgcgccagcccggcctgcagccctga

SEQ ID NO: 16 - DNA, 8556 bp, Homo sapiens
ccccaccctgcaggaggggagaaggggagagatggggttgaagggagagacagagaaaagggagaaccagaggcccagccaggaggacacagacagtgagcctgagagagagacggccggcaagagtaaaggatgcaggaacagccaggcagggtcgggggcagagcaggggagcgcgggccgcggaaagaccgagaaagcaggagacaaggagttgtccttaagggccagaaggaggaacagacagagaaaggggactggggggagggaaagaaaatcacaggcgctgagagggcgcgggggaccgtacgggtctccgggtgtccgcgcatctgtacctgcgcgcgcgtgcgtacctgtggctggcggtgggcacgtgcacgtgctcgggggcagtgctgggggcgggaaagacgcaagaccgccggctgcgggacagatcgaactcgagggccccgacccgggtgacccccgccccctccccgcgcgcgctcccgggccccgagctggttaacgcgccgcccccgccgcgccggctcctccccgccagggcagtgccccggctccgccaacgccctcactagacctggcggccggaccgacccgcgcctggcggatgcgcccggcgcgcccacagcagcccccgcgcccgccgtgccgccgccgggacgtggggcccttgggccgtcgggccgcctggggagcgccagcccggatccggctgcccagatgcgggcgccactctgcctgctcctgctcgtcgcccacgccgtggacatgctcgccctgaaccgaaggaagaagcaaggtacaaggggtggctgggcagggcggccgggcaggcgctgcggggcagaccgggggccggaggaccgggggcggcggctctgggggcatctgcctggtgcctggcgccctgggtccttcgctcctccagacgagtctcaacctcacttaagatggggagattgaggctccagggcacccagtgagcgagccattgagtagggtgggccaaggagactcacccagaagggagagggtagcagggctctctgtagtagccgcatggtcagcagaaaggaggatggcttcgtgcagagacagaagactcaagagccagcctgcggggcacagggactaggagacagactcagtggcgtagaagccattccaagcatccatgctcggccaggaagtgacatagagagccttgggctgggcgtccagaggccccagtcccagcctcatcactcaccctgactcctggtgtcacctcccagagggcagtgatcctccatggcccccctatgttgagttcagccagacctgagacctgagttcaggttcacttattctaataggcctggctgctcccaaggtgccccttaccactgagaactccctcacacgtgccgggaaggatgtgcaggctcttctatcacactcttcccctcagctctgcccttctgtctcctggtctcctttctctgttcaaagaggaatgggaagggggcagattcgcagggctatggacatgagtttatgtccaagcagggcatgagatgctggcaccttcttgcaggcaccccctgtctttgtgagtttctctggcatctcctcacttcgttggcagggttgggaacagaggaaaagtcacagcaccacaactcacttccctaggctgtccacttctttttattttcttctttattggaaacatggcttcactctgtctctccgactggagtacagtggcacaaacacagctcactgcagccttccccgctcaggcgatcctcccaccttagcctcccaagtagctgggactacaggtgtgtgccaccatacccggctaatttttgtattttttgtagagacagggcctcgccatattgtccaggctggtctcaaactcctggactcaagcaattctcccatcttggcctcccaaaatgctgggatgacaggcatgagccactgtgcctggccaggctgacaatccctaactcccatgagtccctccctgcaagtgttcagcttaatcagggtcctggggtgatggtcagggtaacagagactccttatgagcctgggagaggctcacttaagattctagagcattttccacctcttttttccccagctttggctcctgggtgccaattctggggtaacaaaaaattttctccttaaaaaaatttctcccattgctattcctgatgatggtgggagcccttgctgatggtgacagtgagaaatggagtattgctgagtattgttcaggaacccagcaggagggtgagggtctcaccccaccccactcatcatgtgcacccacacacactcaaaaattcacattactttggtaaagattaagtttgcttgtgagtgatggaaaacttcaaataacagtgatttaagcaagatagatgttgatttctctctcatgtaacagtctacacaggcaggctagagctggtacagcagttctgccttcctcaggcacccaggtgctttttatctgatttttccaccgcctcacgggacatagtttccacctcatgatctgaaatggctgcttgtgctccagccatcgttcacattccacccaacagaaaggatgaggggacgacaaggggcacacccctgccctttaaggcagctttcttgaagtgtcacatgctgctttaccttacatctgattggccagatgttggtcacatgaccatatctagttgtaagggaaggtgggaaatgtagtcttttgtttgggtggccatgtgcccagttactatcccaatgagaccatctcagctgctcgcatatatggtttgaccatctctctctccctttccccctccttctgccctggtcttgtgggcagtgggcactggcctggggggcaactgcacaggctgtatcatctgctcagaggagaacggctgttccacctgccagcagaggctcttcctgttcatccgccgggaaggcatccgccagtacggcaagtgcctgcacgactgtccccctgggtacttcggcatccgcggccaggaggtcaacaggtgcaaaaatacgtggcttctcccttgttctatgctagtgctgggctcctagacaccatgggcttagatcccaccctttcaccccagcacagacagaggggaagtaggtgcatgtctaagcccagactctgagatacctgcctgtgtccaggctgactccgcagcccgaggcaagtcagtccgctctcagccttaattagcactaatagaacaggcaactttcacttctaccctagtttgtggtgtggccttaggcatgtaactcaccttctctggtcttagctcaaataggaaaaggagtcctaattccaagctctaatttttgctgtaactttgggcaaattatcactatttctgaggtgaaaaagaagcagggagtcctcactccagccctggtttgcgggtgacttcaagcaagtcactcgctctctctgagcctcccctcaaatgagaaagggagtgttctcttcacatgcagcccagtttgaccttggccaagtctgggcttcaaaggagacactgagtcctgacccaaatgctaccaagcctggagctacccagagggccttccacctcgggcagacccagtgggccccatccttggcagtctccctcacctggggtgtccctggctctattacagaatgtggggccacttgtgagagctgcttcagccaggacttctgcatccggtgcaagaggcagttttacttgtacaaggggaagtgtctgcccacctgcccgccgggcactttggcccaccagaacacacgggagtgccagggtgagtggggacctccccgccctgcccctgcccctcccctctccctggagcgggggcttggtgagagatgttaggggccctggaagaaattacagtagaatgccatatggtgagggaaggcccagcaccaccatgtcaggtacactgggtacctccacatagtaactgcaaaacactagagccaacataaccgccattactatgattactactacaaccaacactggcattgttattaatgccaagggaatcagcagagtgcagaagaagagcgctgatgtttatttgtttgtttgtttgcttgtttgcttgttttgtttgagatagggtctcactctgttgcccagctggagtgcagtggcatgatcatagctcactgcagcctcaaactcctaggctcagagaatcctcccgcctcagcctcccgagtaactgggaccacaggcatgtaccacaacagctggctatgcgtttatgtttttgtaaagcctgtgtcctggtcctgagggcttcccaggcaaccttccagcctcctctcctgtccccgccttcctgagacttgactcccaactcaggctgccccatctcccaaacatggtgggccgggctcccaaggctttgcacacctgcctggaatgggcttccttcttctcacctgacgagctcccctctctccctttaactcatacatcacctcctccaggaagcctttcctttaaagctagacagtcggccgctctggcttcaggcccatattagtctgtgcatgcattcgtttcgctctcattccaggctcttataaataactctgctcctagcaggtggaagtggtgtcttcagtcttcagaggactgaacccctcaaaccctgcccttggatctgaaggaccctcactgccggctgaccctgtctgtctgtctgtctgtctctcctttcaccccacaggggagtgtgaactgggtccctggggcggctggagcccctgcacacacaatggaaagacctgcggctcggcttggggcctggagagccgggtacgagaggctggccgggctgggcatgaggaggcagccacctgccaggtgctttctgagtcaaggaaatgtcccatccagaggccctgcccaggaggtgagccccaggacaggcacacgaggctgcggtgggaaaggcccacagggacagggcggactcagatcactgccccacaaatagtatctatgagactgcctgaaaggccaccattagccatactatcatgtggagtacacatcaccttccctggtgtcctttcaaaggagggccttccttgctgggttcagtcccaaatacattaggaaaattgagctggccaggagttcccaaccctctcaaactgaacaccccccttttaataagaaggatttgggaataccctctttatcattcttaatgaagttcatggagaatgtggcctacttgcaaaggtaatttttttttatcagtatagtgtcctaactgtaatatagaggagaaagaaaagaaatttcttgatggaagcgcttggcccaacataccaacagacacttagggaagtacaatcccagtgaggaccgcggccgtggtcagcgccccaagtacgtggccacatcgccatcagagatgcgatttcccaacagtgaccaatgattagtaaggtccaacccaattgatctctgattgacaccacactcacagtgccctagaatctgtgagtttcgtatacataaagcacttggggctgtggcctgcatacagtgagcgcttgctaaatgctgaagtattgttgccacagtgttatgcagaagttggtgcagggacacagatgaaaggtgtccagcgtccagcacagagcctgctcagctatggaaggagtgtgccggggaaagccatggggactcccatgaggccacccgacatgctgattggggggccccaggtgaacctgcaggcctggccgagccagatgtccagcacaagaaggccctgaacaagttagtggccctcgccactccctgaagacctagagagaaaggttcagtttggggtaccttagcccacggtccaaactctcaacaggagggactgcaaggtcagtgcccaaatgttctgagccccgttgttggggagtgggtggggcacccttgtctttcaggactgaggaggctcccaggacctaactggccctgcagccttggtcaccgggctctgtcctctcattgcagagaggagccccggccagaagaagggcaggaaggaccggcgcccacgcaaggacaggaagctggaccgcaggctggacgtgaggccgcgccagcccggcctgcagccctgaccgccggctctcccgactctctggtcctagtcctcggcccctgcacacctcctcctgctccttctcctcctctcctcttactctttctcctctgtcttctccatttgtcctctctttctttccacccttctatcatttttctgtcagtctaccttccctttctttttcttttttatttcctttatttcttccacctccattctcctctcctttctccctccctccttcccttccttcctcttctttctcacttatcttttatctttccttttctttcttcctgtgtttcttcctgtccttcaccgcatccttctctctctccctcctcttgtctccctctcacacacactttaagagggaccatgagcctgtgccctcccctgcagctttctctatctacaacttaaagaaagcaaacatcttttcccaggcctttccctgaccccatctttgcagagaaagggtttccagagggcaaagctgggacacagcacaggtgaatcctgaaggccctgcttctgctctgggggaggctccaggaccctgagctgtgagcacctggttctctggacagtccccagaggccatttccacagccttcagccaccagccaccccgaggagctggctggacaaggctccagggcttccagaggcctggcttggacacctcccccagctggccgtggagggtcacaacctggcctctgggtgggcagccagccctggagggcatcctctgcaagctgcctgccaccctcatcggcactcccccacaggcctccctctcatgggttccatgcccctttttcccaagccggatcaggtgagctgtcactgctgggggatccacctgcccagcccagaagaggccactgaaacggaaaggaaagctgagattatccagcagctctgttccccacctcagcgcttcctgcccatgtggggaaacaggtctgagaaggaaggggcttgcccagggtcacacaggaagccttcaggctctgcttctgcctgatggctctgctcagcacattcacggtggagaggagaatttgggggtcacttgaggggggaaatgtagggaattgtgggtggggagcaagggaagatccgtgcactcgtccacacccaccaccacactcgctgacacccacccccacacgctgacacccacccccacacttgcccacacccatcaccgcactcgcccacacccaccaccacactgccccacacccaccaccacactcccccacacccaccaccacactcgcccacacccaccaccagtgacttgagcatctgtgcttcgctgtgacgcccctcgccctaggcaggaacgacgctgggaggagtctccaggtcagacccagcttggaagcaagtctgtcctcactgcctatccttctgccatcataacacccccttcctgctctgctccccggaatcctcagaaacgggatttgtatttgccgtgactggttggcctgaacacgtagggctccgtgactgggacaggaatgggcaggagaagcaagagtcggagctccaaggggcccaggggtggcctggggaaggaagatggtcagcaggctgggggagaggctctaggtgatgaaatattacattcccgaccccaagagagcacccaccctcagacctgccctccacctggcagctggggagccctggcctgaacccccccctcccagcaggcccaccctctctctgacttccctgctctcacctccccgagaacagctagagccccctcctccgcctggccaggccaccagcttctcttctgcaaacgtttgtgcctctgaaatgctccgttgttattgtttcaagaccctaacttttttttaaaactttcttaataaagggaaaagaaacttgtaaatgcttcttgagcatcaagagggtgttgcaaaaccatgatactgctgagtttggagtagcagaatttaaaacatgtggagtggttttcacaggaatgcttggggctgagaggggtcagagtgtattggggattggggtggggtttcagcttggggggagctgataaaagaggaggggccctcagcccctccaggctactctcaagaagcagactcagccagaggcagaagagggtgacacctcgatccccagaacctcgcagtttcacgaaccagatgtctcagggaccaggggtacctaggaggttgacagtcccacggggccatctaaacaccctgggctgctggtgagagtggccttggcattgggaggcacaggtgggagctccagcctgtcaccagctatctgatggggtccaggtcaagtcacttccccttccggggcctc

SEQ ID NO: 17 - DNA, 8556 bp, Homo sapiens
ccccaccctgcaggaggggagaaggggagagatggggttgaagggagagacagagaaaagggagaaccagaggcccagccaggaggacacagacagtgagcctgagagagagacggccggcaagagtaaaggatgcaggaacagccaggcagggtcgggggcagagcaggggagcgcgggccgcggaaagaccgagaaagcaggagacaaggagttgtccttaagggccagaaggaggaacagacagagaaaggggactggggggagggaaagaaaatcacaggcgctgagagggcgcgggggaccgtacgggtctccgggtgtccgcgcatctgtacctgcgcgcgcgtgcgtacctgtggctggcggtgggcacgtgcacgtgctcgggggcagtgctgggggcgggaaagacgcaagaccgccggctgcgggacagatcgaactcgagggccccgacccgggtgacccccgccccctccccgcgcgcgctcccgggccccgagctggttaacgcgccgcccccgccgcgccggctcctccccgccagggcagtgccccggctccgccaacgccctcactagacctggcggccggaccgacccgcgcctggcggatgcgcccggcgcgcccacagcagcccccgcgcccgccgtgccgccgccgggacgtggggcccttgggccgtcgggccgcctggggagcgccagcccggatccggctgcccagatgcgggcgccactctgcctgctcctgctcgtcgcccacgccgtggacatgctcgccctgaaccgaaggaagaagcaaggtacaaggggtggctgggcagggcggccgggcaggcgctgcggggcagaccgggggccggaggaccgggggcggcggctctgggggcatctgcctggtgcctggcgccctgggtccttcgctcctccagacgagtctcaacctcacttaagatggggagattgaggctccagggcacccagtgagcgagccattgagtagggtgggccaaggagactcacccagaagggagagggtagcagggctctctgtagtagccgcatggtcagcagaaaggaggatggcttcgtgcagagacagaagactcaagagccagcctgcggggcacagggactaggagacagactcagtggcgtagaagccattccaagcatccatgctcggccaggaagtgacatagagagccttgggctgggcgtccagaggccccagtcccagcctcatcactcaccctgactcctggtgtcacctcccagagggcagtgatcctccatggcccccctatgttgagttcagccagacctgagacctgagttcaggttcacttattctaataggcctggctgctcccaaggtgccccttaccactgagaactccctcacacgtgccgggaaggatgtgcaggctcttctatcacactcttcccctcagctctgcccttctgtctcctggtctcctttctctgttcaaagaggaatgggaagggggcagattcgcagggctatggacatgagtttatgtccaagcagggcatgagatgctggcaccttcttgcaggcaccccctgtctttgtgagtttctctggcatctcctcacttcgttggcagggttgggaacagaggaaaagtcacagcaccacaactcacttccctaggctgtccacttctttttattttcttctttattggaaacatggcttcactctgtctctccgactggagtacagtggcacaaacacagctcactgcagccttccccgctcaggcgatcctcccaccttagcctcccaagtagctgggactacaggtgtgtgccaccatacccggctaatttttgtattttttgtagagacagggcctcgccatattgtccaggctggtctcaaactcctggactcaagcaattctcccatcttggcctcccaaaatgctgggatgacaggcatgagccactgtgcctggccaggctgacaatccctaactcccatgagtccctccctgcaagtgttcagcttaatcagggtcctggggtgatggtcagggtaacagagactccttatgagcctgggagaggctcacttaagattctagagcattttccacctcttttttccccagctttggctcctgggtgccaattctggggtaacaaaaaattttctccttaaaaaaatttctcccattgctattcctgatgatggtgggagcccttgctgatggtgacagtgagaaatggagtattgctgagtattgttcaggaacccagcaggagggtgagggtctcaccccaccccactcatcatgtgcacccacacacactcaaaaattcacattactttggtaaagattaagtttgcttgtgagtgatggaaaacttcaaataacagtgatttaagcaagatagatgttgatttctctctcatgtaacagtctacacaggcaggctagagctggtacagcagttctgccttcctcaggcacccaggtgctttttatctgatttttccaccgcctcacgggacatagtttccacctcatgatctgaaatggctgcttgtgctccagccatcgttcacattccacccaacagaaaggatgaggggacgacaaggggcacacccctgccctttaaggcagctttcttgaagtgtcacatgctgctttaccttacatctgattggccagatgttggtcacatgaccatatctagttgtaagggaaggtgggaaatgtagtcttttgtttgggtggccatgtgcccagttactatcccaatgagaccatctcagctgctcgcatatatggtttgaccatctctctctccctttccccctccttctgccctggtcttgtgggcagtgggcactggcctggggggcaactgcacaggctgtatcatctgctcagaggagaacggctgttccacctgccagcagaggctcttcctgttcatccgccgggaaggcatccgccagtacggcaagtgcctgcacgactgtccccctgggtacttcggcatccgcggccaggaggtcaacaggtgcaaaagtacgtggcttctcccttgttctatgctagtgctgggctcctagacaccatgggcttagatcccaccctttcaccccagcacagacagaggggaagtaggtgcatgtctaagcccagactctgagatacctgcctgtgtccaggctgactccgcagcccgaggcaagtcagtccgctctcagccttaattagcactaatagaacaggcaactttcacttctaccctagtttgtggtgtggccttaggcatgtaactcaccttctctggtcttagctcaaataggaaaaggagtcctaattccaagctctaatttttgctgtaactttgggcaaattatcactatttctgaggtgaaaaagaagcagggagtcctcactccagccctggtttgcgggtgacttcaagcaagtcactcgctctctctgagcctcccctcaaatgagaaagggagtgttctcttcacatgcagcccagtttgaccttggccaagtctgggcttcaaaggagacactgagtcctgacccaaatgctaccaagcctggagctacccagagggccttccacctcgggcagacccagtgggccccatccttggcagtctccctcacctggggtgtccctggctctattacaaaatgtggggccacttgtgagagctgcttcagccaggacttctgcatccggtgcaagaggcagttttacttgtacaaggggaagtgtctgcccacctgcccgccgggcactttggcccaccagaacacacgggagtgccagggtgagtggggacctccccgccctgcccctgcccctcccctctccctggagcgggggcttggtgagagatgttaggggccctggaagaaattacagtagaatgccatatggtgagggaaggcccagcaccaccatgtcaggtacactgggtacctccacatagtaactgcaaaacactagagccaacataaccgccattactatgattactactacaaccaacactggcattgttattaatgccaagggaatcagcagagtgcagaagaagagcgctgatgtttatttgtttgtttgtttgcttgtttgcttgttttgtttgagatagggtctcactctgttgcccagctggagtgcagtggcatgatcatagctcactgcagcctcaaactcctaggctcagagaatcctcccgcctcagcctcccgagtaactgggaccacaggcatgtaccacaacagctggctatgcgtttatgtttttgtaaagcctgtgtcctggtcctgagggcttcccaggcaaccttccagcctcctctcctgtccccgccttcctgagacttgactcccaactcaggctgccccatctcccaaacatggtgggccgggctcccaaggctttgcacacctgcctggaatgggcttccttcttctcacctgacgagctcccctctctccctttaactcatacatcacctcctccaggaagcctttcctttaaagctagacagtcggccgctctggcttcaggcccatattagtctgtgcatgcattcgtttcgctctcattccaggctcttataaataactctgctcctagcaggtggaagtggtgtcttcagtcttcagaggactgaacccctcaaaccctgcccttggatctgaaggaccctcactgccggctgaccctgtctgtctgtctgtctgtctctcctttcaccccacaggggagtgtgaactgggtccctggggcggctggagcccctgcacacacaatggaaagacctgcggctcggcttggggcctggagagccgggtacgagaggctggccgggctgggcatgaggaggcagccacctgccaggtgctttctgagtcaaggaaatgtcccatccagaggccctgcccaggaggtgagccccaggacaggcacacgaggctgcggtgggaaaggcccacagggacagggcggactcagatcactgccccacaaatagtatctatgagactgcctgaaaggccaccattagccatactatcatgtggagtacacatcaccttccctggtgtcctttcaaaggagggccttccttgctgggttcagtcccaaatacattaggaaaattgagctggccaggagttcccaaccctctcaaactgaacaccccccttttaataagaaggatttgggaataccctctttatcattcttaatgaagttcatggagaatgtggcctacttgcaaaggtaatttttttttatcagtatagtgtcctaactgtaatatagaggagaaagaaaagaaatttcttgatggaagcgcttggcccaacataccaacagacacttagggaagtacaatcccagtgaggaccgcggccgtggtcagcgccccaagtacgtggccacatcgccatcagagatgcgatttcccaacagtgaccaatgattagtaaggtccaacccaattgatctctgattgacaccacactcacagtgccctagaatctgtgagtttcgtatacataaagcacttggggctgtggcctgcatacagtgagcgcttgctaaatgctgaagtattgttgccacagtgttatgcagaagttggtgcagggacacagatgaaaggtgtccagcgtccagcacagagcctgctcagctatggaaggagtgtgccggggaaagccatggggactcccatgaggccacccgacatgctgattggggggccccaggtgaacctgcaggcctggccgagccagatgtccagcacaagaaggccctgaacaagttagtggccctcgccactccctgaagacctagagagaaaggttcagtttggggtaccttagcccacggtccaaactctcaacaggagggactgcaaggtcagtgcccaaatgttctgagccccgttgttggggagtgggtggggcacccttgtctttcaggactgaggaggctcccaggacctaactggccctgcagccttggtcaccgggctctgtcctctcattgcagagaggagccccggccagaagaagggcaggaaggaccggcgcccacgcaaggacaggaagctggaccgcaggctggacgtgaggccgcgccagcccggcctgcagccctgaccgccggctctcccgactctctggtcctagtcctcggcccctgcacacctcctcctgctccttctcctcctctcctcttactctttctcctctgtcttctccatttgtcctctctttctttccacccttctatcatttttctgtcagtctaccttccctttctttttcttttttatttcctttatttcttccacctccattctcctctcctttctccctccctccttcccttccttcctcttctttctcacttatcttttatctttccttttctttcttcctgtgtttcttcctgtccttcaccgcatccttctctctctccctcctcttgtctccctctcacacacactttaagagggaccatgagcctgtgccctcccctgcagctttctctatctacaacttaaagaaagcaaacatcttttcccaggcctttccctgaccccatctttgcagagaaagggtttccagagggcaaagctgggacacagcacaggtgaatcctgaaggccctgcttctgctctgggggaggctccaggaccctgagctgtgagcacctggttctctggacagtccccagaggccatttccacagccttcagccaccagccaccccgaggagctggctggacaaggctccagggcttccagaggcctggcttggacacctcccccagctggccgtggagggtcacaacctggcctctgggtgggcagccagccctggagggcatcctctgcaagctgcctgccaccctcatcggcactcccccacaggcctccctctcatgggttccatgcccctttttcccaagccggatcaggtgagctgtcactgctgggggatccacctgcccagcccagaagaggccactgaaacggaaaggaaagctgagattatccagcagctctgttccccacctcagcgcttcctgcccatgtggggaaacaggtctgagaaggaaggggcttgcccagggtcacacaggaagccttcaggctctgcttctgcctgatggctctgctcagcacattcacggtggagaggagaatttgggggtcacttgaggggggaaatgtagggaattgtgggtggggagcaagggaagatccgtgcactcgtccacacccaccaccacactcgctgacacccacccccacacgctgacacccacccccacacttgcccacacccatcaccgcactcgcccacacccaccaccacactgccccacacccaccaccacactcccccacacccaccaccacactcgcccacacccaccaccagtgacttgagcatctgtgcttcgctgtgacgcccctcgccctaggcaggaacgacgctgggaggagtctccaggtcagacccagcttggaagcaagtctgtcctcactgcctatccttctgccatcataacacccccttcctgctctgctccccggaatcctcagaaacgggatttgtatttgccgtgactggttggcctgaacacgtagggctccgtgactgggacaggaatgggcaggagaagcaagagtcggagctccaaggggcccaggggtggcctggggaaggaagatggtcagcaggctgggggagaggctctaggtgatgaaatattacattcccgaccccaagagagcacccaccctcagacctgccctccacctggcagctggggagccctggcctgaacccccccctcccagcaggcccaccctctctctgacttccctgctctcacctccccgagaacagctagagccccctcctccgcctggccaggccaccagcttctcttctgcaaacgtttgtgcctctgaaatgctccgttgttattgtttcaagaccctaacttttttttaaaactttcttaataaagggaaaagaaacttgtaaatgcttcttgagcatcaagagggtgttgcaaaaccatgatactgctgagtttggagtagcagaatttaaaacatgtggagtggttttcacaggaatgcttggggctgagaggggtcagagtgtattggggattggggtggggtttcagcttggggggagctgataaaagaggaggggccctcagcccctccaggctactctcaagaagcagactcagccagaggcagaagagggtgacacctcgatccccagaacctcgcagtttcacgaaccagatgtctcagggaccaggggtacctaggaggttgacagtcccacggggccatctaaacaccctgggctgctggtgagagtggccttggcattgggaggcacaggtgggagctccagcctgtcaccagctatctgatggggtccaggtcaagtcacttccccttccggggcctc

SEQ ID NO: 18 - DNA, 2722 bp, Homo sapiens
cacagcagcccccgcgcccgccgtgccgccgccgggacgtggggcccttgggccgtcgggccgcctggggagcgccagcccggatccggctgcccagatgcgggcgccactctgcctgctcctgctcgtcgcccacgccgtggacatgctcgccctgaaccgaaggaagaagcaagtgggcactggcctggggggcaactgcacaggctgtatcatctgctcagaggagaacggctgttccacctgccagcagaggctcttcctgttcatccgccgggaaggcatccgccagtacggcaagtgcctgcacgactgtccccctgggtacttcggcatccgcggccaggaggtcaacaggtgcaaaaaatgtggggccacttgtgagagctgcttcagccaggacttctgcatccggtgcaagaggcagttttacttgtacaaggggaagtgtctgcccacctgcccgccgggcactttggcccaccagaacacacgggagtgccagggggagtgtgaactgggtccctggggcggctggagcccctgcacacacaatggaaagacctgcggctcggcttggggcctggagagccgggtacgagaggctggccgggctgggcatgaggaggcagccacctgccaggtgctttctgagtcaaggaaatgtcccatccagaggccctgcccaggagagaggagccccggccagaagaagggcaggaaggaccggcgcccacgcaaggacaggaagctggaccgcaggctggacgtgaggccgcgccagcccggcctgcagccctgaccgccggctctcccgactctctggtcctagtcctcggcccctgcacacctcctcctgctccttctcctcctctcctcttactctttctcctctgtcttctccatttgtcctctctttctttccacccttctatcatttttctgtcagtctaccttccctttctttttcttttttatttcctttatttcttccacctccattctcctctcctttctccctccctccttcccttccttcctcttctttctcacttatcttttatctttccttttctttcttcctgtgtttcttcctgtccttcaccgcatccttctctctctccctcctcttgtctccctctcacacacactttaagagggaccatgagcctgtgccctcccctgcagctttctctatctacaacttaaagaaagcaaacatcttttcccaggcctttccctgaccccatctttgcagagaaagggtttccagagggcaaagctgggacacagcacaggtgaatcctgaaggccctgcttctgctctgggggaggctccaggaccctgagctgtgagcacctggttctctggacagtccccagaggccatttccacagccttcagccaccagccaccccgaggagctggctggacaaggctccagggcttccagaggcctggcttggacacctcccccagctggccgtggagggtcacaacctggcctctgggtgggcagccagccctggagggcatcctctgcaagctgcctgccaccctcatcggcactcccccacaggcctccctctcatgggttccatgcccctttttcccaagccggatcaggtgagctgtcactgctgggggatccacctgcccagcccagaagaggccactgaaacggaaaggaaagctgagattatccagcagctctgttccccacctcagcgcttcctgcccatgtggggaaacaggtctgagaaggaaggggcttgcccagggtcacacaggaagccttcaggctctgcttctgcctgatggctctgctcagcacattcacggtggagaggagaatttgggggtcacttgaggggggaaatgtagggaattgtgggtggggagcaagggaagatccgtgcactcgtccacacccaccaccacactcgctgacacccacccccacacgctgacacccacccccacacttgcccacacccatcaccgcactcgcccacacccaccaccacactgccccacacccaccaccacactcccccacacccaccaccacactcgcccacacccaccaccagtgacttgagcatctgtgcttcgctgtgacgcccctcgccctaggcaggaacgacgctgggaggagtctccaggtcagacccagcttggaagcaagtctgtcctcactgcctatccttctgccatcataacacccccttcctgctctgctccccggaatcctcagaaacgggatttgtatttgccgtgactggttggcctgaacacgtagggctccgtgactgggacaggaatgggcaggagaagcaagagtcggagctccaaggggcccaggggtggcctggggaaggaagatggtcagcaggctgggggagaggctctaggtgatgaaatattacattcccgaccccaagagagcacccaccctcagacctgccctccacctggcagctggggagccctggcctgaacccccccctcccagcaggcccaccctctctctgacttccctgctctcacctccccgagaacagctagagccccctcctccgcctggccaggccaccagcttctcttctgcaaacgtttgtgcctctgaaatgctccgttgttattgtttcaagaccctaacttttttttaaaactttcttaataaagggaaaagaaacttgtaaaaaaaaaaaaaaaaaa

SEQ ID NO: 19 - DNA, 8556 bp, Homo sapiens
ccccaccctgcaggaggggagaaggggagagatggggttgaagggagagacagagaaaagggagaaccagaggcccagccaggaggacacagacagtgagcctgagagagagacggccggcaagagtaaaggatgcaggaacagccaggcagggtcgggggcagagcaggggagcgcgggccgcggaaagaccgagaaagcaggagacaaggagttgtccttaagggccagaaggaggaacagacagagaaaggggactggggggagggaaagaaaatcacaggcgctgagagggcgcgggggaccgtacgggtctccgggtgtccgcgcatctgtacctgcgcgcgcgtgcgtacctgtggctggcggtgggcacgtgcacgtgctcgggggcagtgctgggggcgggaaagacgcaagaccgccggctgcgggacagatcgaactcgagggccccgacccgggtgacccccgccccctccccgcgcgcgctcccgggccccgagctggttaacgcgccgcccccgccgcgccggctcctccccgccagggcagtgccccggctccgccaacgccctcactagacctggcggccggaccgacccgcgcctggcggatgcgcccggcgcgcccacagcagcccccgcgcccgccgtgccgccgccgggacgtggggcccttgggccgtcgggccgcctggggagcgccagcccggatccggctgcccagatgcgggcgccactctgcctgctcctgctcgtcgcccacgccgtggacatgctcgccctgaaccgaaggaagaagcaaggtacaaggggtggctgggcagggcggccgggcaggcgctgcggggcagaccgggggccggaggaccgggggcggcggctctgggggcatctgcctggtgcctggcgccctgggtccttcgctcctccagacgagtctcaacctcacttaagatggggagattgaggctccagggcacccagtgagcgagccattgagtagggtgggccaaggagactcacccagaagggagagggtagcagggctctctgtagtagccgcatggtcagcagaaaggaggatggcttcgtgcagagacagaagactcaagagccagcctgcggggcacagggactaggagacagactcagtggcgtagaagccattccaagcatccatgctcggccaggaagtgacatagagagccttgggctgggcgtccagaggccccagtcccagcctcatcactcaccctgactcctggtgtcacctcccagagggcagtgatcctccatggcccccctatgttgagttcagccagacctgagacctgagttcaggttcacttattctaataggcctggctgctcccaaggtgccccttaccactgagaactccctcacacgtgccgggaaggatgtgcaggctcttctatcacactcttcccctcagctctgcccttctgtctcctggtctcctttctctgttcaaagaggaatgggaagggggcagattcgcagggctatggacatgagtttatgtccaagcagggcatgagatgctggcaccttcttgcaggcaccccctgtctttgtgagtttctctggcatctcctcacttcgttggcagggttgggaacagaggaaaagtcacagcaccacaactcacttccctaggctgtccacttctttttattttcttctttattggaaacatggcttcactctgtctctccgactggagtacagtggcacaaacacagctcactgcagccttccccgctcaggcgatcctcccaccttagcctcccaagtagctgggactacaggtgtgtgccaccatacccggctaatttttgtattttttgtagagacagggcctcgccatattgtccaggctggtctcaaactcctggactcaagcaattctcccatcttggcctcccaaaatgctgggatgacaggcatgagccactgtgcctggccaggctgacaatccctaactcccatgagtccctccctgcaagtgttcagcttaatcagggtcctggggtgatggtcagggtaacagagactccttatgagcctgggagaggctcacttaagattctagagcattttccacctcttttttccccagctttggctcctgggtgccaattctggggtaacaaaaaattttctccttaaaaaaatttctcccattgctattcctgatgatggtgggagcccttgctgatggtgacagtgagaaatggagtattgctgagtattgttcaggaacccagcaggagggtgagggtctcaccccaccccactcatcatgtgcacccacacacactcaaaaattcacattactttggtaaagattaagtttgcttgtgagtgatggaaaacttcaaataacagtgatttaagcaagatagatgttgatttctctctcatgtaacagtctacacaggcaggctagagctggtacagcagttctgccttcctcaggcacccaggtgctttttatctgatttttccaccgcctcacgggacatagtttccacctcatgatctgaaatggctgcttgtgctccagccatcgttcacattccacccaacagaaaggatgaggggacgacaaggggcacacccctgccctttaaggcagctttcttgaagtgtcacatgctgctttaccttacatctgattggccagatgttggtcacatgaccatatctagttgtaagggaaggtgggaaatgtagtcttttgtttgggtggccatgtgcccagttactatcccaatgagaccatctcagctgctcgcatatatggtttgaccatctctctctccctttccccctccttctgccctggtcttgtgggcagtgggcactggcctggggggcaactgcacaggctgtatcatctgctcagaggagaacggctgttccacctgccagcagaggctcttcctgttcatccgccgggaaggcatccgccagtacggcaagtgcctgcacgactgtccccctgggtacttcggcatccgcggccaggaggtcaacaggtgcaaaagtacgtggcttctcccttgttctatgctagtgctgggctcctagacaccatgggcttagatcccaccctttcaccccagcacagacagaggggaagtaggtgcatgtctaagcccagactctgagatacctgcctgtgtccaggctgactccgcagcccgaggcaagtcagtccgctctcagccttaattagcactaatagaacaggcaactttcacttctaccctagtttgtggtgtggccttaggcatgtaactcaccttctctggtcttagctcaaataggaaaaggagtcctaattccaagctctaatttttgctgtaactttgggcaaattatcactatttctgaggtgaaaaagaagcagggagtcctcactccagccctggtttgcgggtgacttcaagcaagtcactcgctctctctgagcctcccctcaaatgagaaagggagtgttctcttcacatgcagcccagtttgaccttggccaagtctgggcttcaaaggagacactgagtcctgacccaaatgctaccaagcctggagctacccagagggccttccacctcgggcagacccagtgggccccatccttggcagtctccctcacctggggtgtccctggctctattacagaatgtggggccacttgtgagagctgcttcagccaggacttctgcatccggtgcaagaggcagttttacttgtacaaggggaagtgtctgcccacctgcccgccgggcactttggcccaccagaacacacgggagtgccagggtgagtggggacctccccgccctgcccctgcccctcccctctccctggagcgggggcttggtgagagatgttaggggccctggaagaaattacagtagaatgccatatggtgagggaaggcccagcaccaccatgtcaggtacactgggtacctccacatagtaactgcaaaacactagagccaacataaccgccattactatgattactactacaaccaacactggcattgttattaatgccaagggaatcagcagagtgcagaagaagagcgctgatgtttatttgtttgtttgtttgcttgtttgcttgttttgtttgagatagggtctcactctgttgcccagctggagtgcagtggcatgatcatagctcactgcagcctcaaactcctaggctcagagaatcctcccgcctcagcctcccgagtaactgggaccacaggcatgtaccacaacagctggctatgcgtttatgtttttgtaaagcctgtgtcctggtcctgagggcttcccaggcaaccttccagcctcctctcctgtccccgccttcctgagacttgactcccaactcaggctgccccatctcccaaacatggtgggccgggctcccaaggctttgcacacctgcctggaatgggcttccttcttctcacctgacgagctcccctctctccctttaactcatacatcacctcctccaggaagcctttcctttaaagctagacagtcggccgctctggcttcaggcccatattagtctgtgcatgcattcgtttcgctctcattccaggctcttataaataactctgctcctagcaggtggaagtggtgtcttcagtcttcagaggactgaacccctcaaaccctgcccttggatctgaaggaccctcactgccggctgaccctgtctgtctgtctgtctgtctctcctttcaccccacaggggagtgtgaactgggtccctggggcggctggagcccctgcacacacaatggaaagacctgcggctcggcttggggcctggagagccgggtacgagaggctggccgggctgggcatgaggaggcagccacctgccaggtgctttctgagtcaaggaaatgtcccatccagaggccctgcccaggaggtgagccccaggacaggcacacgaggctgcggtgggaaaggcccacagggacagggcggactcagatcactgccccacaaatagtatctatgagactgcctgaaaggccaccattagccatactatcatgtggagtacacatcaccttccctggtgtcctttcaaaggagggccttccttgctgggttcagtcccaaatacattaggaaaattgagctggccaggagttcccaaccctctcaaactgaacaccccccttttaataagaaggatttgggaataccctctttatcattcttaatgaagttcatggagaatgtggcctacttgcaaaggtaatttttttttatcagtatagtgtcctaactgtaatatagaggagaaagaaaagaaatttcttgatggaagcgcttggcccaacataccaacagacacttagggaagtacaatcccagtgaggaccgcggccgtggtcagcgccccaagtacgtggccacatcgccatcagagatgcgatttcccaacagtgaccaatgattagtaaggtccaacccaattgatctctgattgacaccacactcacagtgccctagaatctgtgagtttcgtatacataaagcacttggggctgtggcctgcatacagtgagcgcttgctaaatgctgaagtattgttgccacagtgttatgcagaagttggtgcagggacacagatgaaaggtgtccagcgtccagcacagagcctgctcagctatggaaggagtgtgccggggaaagccatggggactcccatgaggccacccgacatgctgattggggggccccaggtgaacctgcaggcctggccgagccagatgtccagcacaagaaggccctgaacaagttagtggccctcgccactccctgaagacctagagagaaaggttcagtttggggtaccttagcccacggtccaaactctcaacaggagggactgcaaggtcagtgcccaaatgttctgagccccgttgttggggagtgggtggggcacccttgtctttcaggactgaggaggctcccaggacctaactggccctgcagccttggtcaccgggctctgtcctctcattgcagagaggagccccggccagaagaagggcaggaaggaccggcgcccacgcaaggacaggaagctggaccgcaggctggacgtgaggccgcgccagcccggcctgcagccctgaccgccggctctcccgactctctggtcctagtcctcggcccctgcacacctcctcctgctccttctcctcctctcctcttactctttctcctctgtcttctccatttgtcctctctttctttccacccttctatcatttttctgtcagtctaccttccctttctttttcttttttatttcctttatttcttccacctccattctcctctcctttctccctccctccttcccttccttcctcttctttctcacttatcttttatctttccttttctttcttcctgtgtttcttcctgtccttcaccgcatccttctctctctccctcctcttgtctccctctcacacacactttaagagggaccatgagcctgtgccctcccctgcagctttctctatctacaacttaaagaaagcaaacatcttttcccaggcctttccctgaccccatctttgcagagaaagggtttccagagggcaaagctgggacacagcacaggtgaatcctgaaggccctgcttctgctctgggggaggctccaggaccctgagctgtgagcacctggttctctggacagtccccagaggccatttccacagccttcagccaccagccaccccgaggagctggctggacaaggctccagggcttccagaggcctggcttggacacctcccccagctggccgtggagggtcacaacctggcctctgggtgggcagccagccctggagggcatcctctgcaagctgcctgccaccctcatcggcactcccccacaggcctccctctcatgggttccatgcccctttttcccaagccggatcaggtgagctgtcactgctgggggatccacctgcccagcccagaagaggccactgaaacggaaaggaaagctgagattatccagcagctctgttccccacctcagcgcttcctgcccatgtggggaaacaggtctgagaaggaaggggcttgcccagggtcacacaggaagccttcaggctctgcttctgcctgatggctctgctcagcacattcacggtggagaggagaatttgggggtcacttgaggggggaaatgtagggaattgtgggtggggagcaagggaagatccgtgcactcgtccacacccaccaccacactcgctgacacccacccccacacgctgacacccacccccacacttgcccacacccatcaccgcactcgcccacacccaccaccacactgccccacacccaccaccacactcccccacacccaccaccacactcgcccacacccaccaccagtgacttgagcatctgtgcttcgctgtgacgcccctcgccctaggcaggaacgacgctgggaggagtctccaggtcagacccagcttggaagcaagtctgtcctcactgcctatccttctgccatcataacacccccttcctgctctgctccccggaatcctcagaaacgggatttgtatttgccgtgactggttggcctgaacacgtagggctccgtgactgggacaggaatgggcaggagaagcaagagtcggagctccaaggggcccaggggtggcctggggaaggaagatggtcagcaggctgggggagaggctctaggtgatgaaatattacattcccgaccccaagagagcacccaccctcagacctgccctccacctggcagctggggagccctggcctgaacccccccctcccagcaggcccaccctctctctgacttccctgctctcacctccccgagaacagctagagccccctcctccgcctggccaggccaccagcttctcttctgcaaacgtttgtgcctctgaaatgctccgttgttattgtttcaagaccctaacttttttttaaaactttcttaataaagggaaaagaaacttgtaaatgcttcttgagcatcaagagggtgttgcaaaaccatgatactgctgagtttggagtagcagaatttaaaacatgtggagtggttttcacaggaatgcttggggctgagaggggtcagagtgtattggggattggggtggggtttcagcttggggggagctgataaaagaggaggggccctcagcccctccaggctactctcaagaagcagactcagccagaggcagaagagggtgacacctcgatccccagaacctcgcagtttcacgaaccagatgtctcagggaccaggggtacctaggaggttgacagtcccacggggccatctaaacaccctgggctgctggtgagagtggccttggcattgggaggcacaggtgggagctccagcctgtcaccagctatctgatggggtccaggtcaagtcacttccccttccggggcctc

SEQ ID NO: 20 - DNA, 8556 bp, Homo sapiens
ccccaccctgcaggaggggagaaggggagagatggggttgaagggagagacagagaaaagggagaaccagaggcccagccaggaggacacagacagtgagcctgagagagagacggccggcaagagtaaaggatgcaggaacagccaggcagggtcgggggcagagcaggggagcgcgggccgcggaaagaccgagaaagcaggagacaaggagttgtccttaagggccagaaggaggaacagacagagaaaggggactggggggagggaaagaaaatcacaggcgctgagagggcgcgggggaccgtacgggtctccgggtgtccgcgcatctgtacctgcgcgcgcgtgcgtacctgtggctggcggtgggcacgtgcacgtgctcgggggcagtgctgggggcgggaaagacgcaagaccgccggctgcgggacagatcgaactcgagggccccgacccgggtgacccccgccccctccccgcgcgcgctcccgggccccgagctggttaacgcgccgcccccgccgcgccggctcctccccgccagggcagtgccccggctccgccaacgccctcactagacctggcggccggaccgacccgcgcctggcggatgcgcccggcgcgcccacagcagcccccgcgcccgccgtgccgccgccgggacgtggggcccttgggccgtcgggccgcctggggagcgccagcccggatccggctgcccagatgcgggcgccactctgcctgctcctgctcgtcgcccacgccgtggacatgctcgccctgaaccgaaggaagaagcaagatacaaggggtggctgggcagggcggccgggcaggcgctgcggggcagaccgggggccggaggaccgggggcggcggctctgggggcatctgcctggtgcctggcgccctgggtccttcgctcctccagacgagtctcaacctcacttaagatggggagattgaggctccagggcacccagtgagcgagccattgagtagggtgggccaaggagactcacccagaagggagagggtagcagggctctctgtagtagccgcatggtcagcagaaaggaggatggcttcgtgcagagacagaagactcaagagccagcctgcggggcacagggactaggagacagactcagtggcgtagaagccattccaagcatccatgctcggccaggaagtgacatagagagccttgggctgggcgtccagaggccccagtcccagcctcatcactcaccctgactcctggtgtcacctcccagagggcagtgatcctccatggcccccctatgttgagttcagccagacctgagacctgagttcaggttcacttattctaataggcctggctgctcccaaggtgccccttaccactgagaactccctcacacgtgccgggaaggatgtgcaggctcttctatcacactcttcccctcagctctgcccttctgtctcctggtctcctttctctgttcaaagaggaatgggaagggggcagattcgcagggctatggacatgagtttatgtccaagcagggcatgagatgctggcaccttcttgcaggcaccccctgtctttgtgagtttctctggcatctcctcacttcgttggcagggttgggaacagaggaaaagtcacagcaccacaactcacttccctaggctgtccacttctttttattttcttctttattggaaacatggcttcactctgtctctccgactggagtacagtggcacaaacacagctcactgcagccttccccgctcaggcgatcctcccaccttagcctcccaagtagctgggactacaggtgtgtgccaccatacccggctaatttttgtattttttgtagagacagggcctcgccatattgtccaggctggtctcaaactcctggactcaagcaattctcccatcttggcctcccaaaatgctgggatgacaggcatgagccactgtgcctggccaggctgacaatccctaactcccatgagtccctccctgcaagtgttcagcttaatcagggtcctggggtgatggtcagggtaacagagactccttatgagcctgggagaggctcacttaagattctagagcattttccacctcttttttccccagctttggctcctgggtgccaattctggggtaacaaaaaattttctccttaaaaaaatttctcccattgctattcctgatgatggtgggagcccttgctgatggtgacagtgagaaatggagtattgctgagtattgttcaggaacccagcaggagggtgagggtctcaccccaccccactcatcatgtgcacccacacacactcaaaaattcacattactttggtaaagattaagtttgcttgtgagtgatggaaaacttcaaataacagtgatttaagcaagatagatgttgatttctctctcatgtaacagtctacacaggcaggctagagctggtacagcagttctgccttcctcaggcacccaggtgctttttatctgatttttccaccgcctcacgggacatagtttccacctcatgatctgaaatggctgcttgtgctccagccatcgttcacattccacccaacagaaaggatgaggggacgacaaggggcacacccctgccctttaaggcagctttcttgaagtgtcacatgctgctttaccttacatctgattggccagatgttggtcacatgaccatatctagttgtaagggaaggtgggaaatgtagtcttttgtttgggtggccatgtgcccagttactatcccaatgagaccatctcagctgctcgcatatatggtttgaccatctctctctccctttccccctccttctgccctggtcttgtgggcagtgggcactggcctggggggcaactgcacaggctgtatcatctgctcagaggagaacggctgttccacctgccagcagaggctcttcctgttcatccgccgggaaggcatccgccagtacggcaagtgcctgcacgactgtccccctgggtacttcggcatccgcggccaggaggtcaacaggtgcaaaagtacgtggcttctcccttgttctatgctagtgctgggctcctagacaccatgggcttagatcccaccctttcaccccagcacagacagaggggaagtaggtgcatgtctaagcccagactctgagatacctgcctgtgtccaggctgactccgcagcccgaggcaagtcagtccgctctcagccttaattagcactaatagaacaggcaactttcacttctaccctagtttgtggtgtggccttaggcatgtaactcaccttctctggtcttagctcaaataggaaaaggagtcctaattccaagctctaatttttgctgtaactttgggcaaattatcactatttctgaggtgaaaaagaagcagggagtcctcactccagccctggtttgcgggtgacttcaagcaagtcactcgctctctctgagcctcccctcaaatgagaaagggagtgttctcttcacatgcagcccagtttgaccttggccaagtctgggcttcaaaggagacactgagtcctgacccaaatgctaccaagcctggagctacccagagggccttccacctcgggcagacccagtgggccccatccttggcagtctccctcacctggggtgtccctggctctattacagaatgtggggccacttgtgagagctgcttcagccaggacttctgcatccggtgcaagaggcagttttacttgtacaaggggaagtgtctgcccacctgcccgccgggcactttggcccaccagaacacacgggagtgccagggtgagtggggacctccccgccctgcccctgcccctcccctctccctggagcgggggcttggtgagagatgttaggggccctggaagaaattacagtagaatgccatatggtgagggaaggcccagcaccaccatgtcaggtacactgggtacctccacatagtaactgcaaaacactagagccaacataaccgccattactatgattactactacaaccaacactggcattgttattaatgccaagggaatcagcagagtgcagaagaagagcgctgatgtttatttgtttgtttgtttgcttgtttgcttgttttgtttgagatagggtctcactctgttgcccagctggagtgcagtggcatgatcatagctcactgcagcctcaaactcctaggctcagagaatcctcccgcctcagcctcccgagtaactgggaccacaggcatgtaccacaacagctggctatgcgtttatgtttttgtaaagcctgtgtcctggtcctgagggcttcccaggcaaccttccagcctcctctcctgtccccgccttcctgagacttgactcccaactcaggctgccccatctcccaaacatggtgggccgggctcccaaggctttgcacacctgcctggaatgggcttccttcttctcacctgacgagctcccctctctccctttaactcatacatcacctcctccaggaagcctttcctttaaagctagacagtcggccgctctggcttcaggcccatattagtctgtgcatgcattcgtttcgctctcattccaggctcttataaataactctgctcctagcaggtggaagtggtgtcttcagtcttcagaggactgaacccctcaaaccctgcccttggatctgaaggaccctcactgccggctgaccctgtctgtctgtctgtctgtctctcctttcaccccacaggggagtgtgaactgggtccctggggcggctggagcccctgcacacacaatggaaagacctgcggctcggcttggggcctggagagccgggtacgagaggctggccgggctgggcatgaggaggcagccacctgccaggtgctttctgagtcaaggaaatgtcccatccagaggccctgcccaggaggtgagccccaggacaggcacacgaggctgcggtgggaaaggcccacagggacagggcggactcagatcactgccccacaaatagtatctatgagactgcctgaaaggccaccattagccatactatcatgtggagtacacatcaccttccctggtgtcctttcaaaggagggccttccttgctgggttcagtcccaaatacattaggaaaattgagctggccaggagttcccaaccctctcaaactgaacaccccccttttaataagaaggatttgggaataccctctttatcattcttaatgaagttcatggagaatgtggcctacttgcaaaggtaatttttttttatcagtatagtgtcctaactgtaatatagaggagaaagaaaagaaatttcttgatggaagcgcttggcccaacataccaacagacacttagggaagtacaatcccagtgaggaccgcggccgtggtcagcgccccaagtacgtggccacatcgccatcagagatgcgatttcccaacagtgaccaatgattagtaaggtccaacccaattgatctctgattgacaccacactcacagtgccctagaatctgtgagtttcgtatacataaagcacttggggctgtggcctgcatacagtgagcgcttgctaaatgctgaagtattgttgccacagtgttatgcagaagttggtgcagggacacagatgaaaggtgtccagcgtccagcacagagcctgctcagctatggaaggagtgtgccggggaaagccatggggactcccatgaggccacccgacatgctgattggggggccccaggtgaacctgcaggcctggccgagccagatgtccagcacaagaaggccctgaacaagttagtggccctcgccactccctgaagacctagagagaaaggttcagtttggggtaccttagcccacggtccaaactctcaacaggagggactgcaaggtcagtgcccaaatgttctgagccccgttgttggggagtgggtggggcacccttgtctttcaggactgaggaggctcccaggacctaactggccctgcagccttggtcaccgggctctgtcctctcattgcagagaggagccccggccagaagaagggcaggaaggaccggcgcccacgcaaggacaggaagctggaccgcaggctggacgtgaggccgcgccagcccggcctgcagccctgaccgccggctctcccgactctctggtcctagtcctcggcccctgcacacctcctcctgctccttctcctcctctcctcttactctttctcctctgtcttctccatttgtcctctctttctttccacccttctatcatttttctgtcagtctaccttccctttctttttcttttttatttcctttatttcttccacctccattctcctctcctttctccctccctccttcccttccttcctcttctttctcacttatcttttatctttccttttctttcttcctgtgtttcttcctgtccttcaccgcatccttctctctctccctcctcttgtctccctctcacacacactttaagagggaccatgagcctgtgccctcccctgcagctttctctatctacaacttaaagaaagcaaacatcttttcccaggcctttccctgaccccatctttgcagagaaagggtttccagagggcaaagctgggacacagcacaggtgaatcctgaaggccctgcttctgctctgggggaggctccaggaccctgagctgtgagcacctggttctctggacagtccccagaggccatttccacagccttcagccaccagccaccccgaggagctggctggacaaggctccagggcttccagaggcctggcttggacacctcccccagctggccgtggagggtcacaacctggcctctgggtgggcagccagccctggagggcatcctctgcaagctgcctgccaccctcatcggcactcccccacaggcctccctctcatgggttccatgcccctttttcccaagccggatcaggtgagctgtcactgctgggggatccacctgcccagcccagaagaggccactgaaacggaaaggaaagctgagattatccagcagctctgttccccacctcagcgcttcctgcccatgtggggaaacaggtctgagaaggaaggggcttgcccagggtcacacaggaagccttcaggctctgcttctgcctgatggctctgctcagcacattcacggtggagaggagaatttgggggtcacttgaggggggaaatgtagggaattgtgggtggggagcaagggaagatccgtgcactcgtccacacccaccaccacactcgctgacacccacccccacacgctgacacccacccccacacttgcccacacccatcaccgcactcgcccacacccaccaccacactgccccacacccaccaccacactcccccacacccaccaccacactcgcccacacccaccaccagtgacttgagcatctgtgcttcgctgtgacgcccctcgccctaggcaggaacgacgctgggaggagtctccaggtcagacccagcttggaagcaagtctgtcctcactgcctatccttctgccatcataacacccccttcctgctctgctccccggaatcctcagaaacgggatttgtatttgccgtgactggttggcctgaacacgtagggctccgtgactgggacaggaatgggcaggagaagcaagagtcggagctccaaggggcccaggggtggcctggggaaggaagatggtcagcaggctgggggagaggctctaggtgatgaaatattacattcccgaccccaagagagcacccaccctcagacctgccctccacctggcagctggggagccctggcctgaacccccccctcccagcaggcccaccctctctctgacttccctgctctcacctccccgagaacagctagagccccctcctccgcctggccaggccaccagcttctcttctgcaaacgtttgtgcctctgaaatgctccgttgttattgtttcaagaccctaacttttttttaaaactttcttaataaagggaaaagaaacttgtaaatgcttcttgagcatcaagagggtgttgcaaaaccatgatactgctgagtttggagtagcagaatttaaaacatgtggagtggttttcacaggaatgcttggggctgagaggggtcagagtgtattggggattggggtggggtttcagcttggggggagctgataaaagaggaggggccctcagcccctccaggctactctcaagaagcagactcagccagaggcagaagagggtgacacctcgatccccagaacctcgcagtttcacgaaccagatgtctcagggaccaggggtacctaggaggttgacagtcccacggggccatctaaacaccctgggctgctggtgagagtggccttggcattgggaggcacaggtgggagctccagcctgtcaccagctatctgatggggtccaggtcaagtcacttccccttccggggcctc

SEQ ID NO: 21 - DNA, 8556 bp, Homo sapiens
ccccaccctgcaggaggggagaaggggagagatggggttgaagggagagacagagaaaagggagaaccagaggcccagccaggaggacacagacagtgagcctgagagagagacggccggcaagagtaaaggatgcaggaacagccaggcagggtcgggggcagagcaggggagcgcgggccgcggaaagaccgagaaagcaggagacaaggagttgtccttaagggccagaaggaggaacagacagagaaaggggactggggggagggaaagaaaatcacaggcgctgagagggcgcgggggaccgtacgggtctccgggtgtccgcgcatctgtacctgcgcgcgcgtgcgtacctgtggctggcggtgggcacgtgcacgtgctcgggggcagtgctgggggcgggaaagacgcaagaccgccggctgcgggacagatcgaactcgagggccccgacccgggtgacccccgccccctccccgcgcgcgctcccgggccccgagctggttaacgcgccgcccccgccgcgccggctcctccccgccagggcagtgccccggctccgccaacgccctcactagacctggcggccggaccgacccgcgcctggcggatgcgcccggcgcgcccacagcagcccccgcgcccgccgtgccgccgccgggacgtggggcccttgggccgtcgggccgcctggggagcgccagcccggatccggctgcccagatgcgggcgccactctgcctgctcctgctcgtcgcccacgccgtggacatgctcgccctgaaccgaaggaagaagcaaggtacaaggggtggctgggcagggcggccgggcaggcgctgcggggcagaccgggggccggaggaccgggggcggcggctctgggggcatctgcctggtgcctggcgccctgggtccttcgctcctccagacgagtctcaacctcacttaagatggggagattgaggctccagggcacccagtgagcgagccattgagtagggtgggccaaggagactcacccagaagggagagggtagcagggctctctgtagtagccgcatggtcagcagaaaggaggatggcttcgtgcagagacagaagactcaagagccagcctgcggggcacagggactaggagacagactcagtggcgtagaagccattccaagcatccatgctcggccaggaagtgacatagagagccttgggctgggcgtccagaggccccagtcccagcctcatcactcaccctgactcctggtgtcacctcccagagggcagtgatcctccatggcccccctatgttgagttcagccagacctgagacctgagttcaggttcacttattctaataggcctggctgctcccaaggtgccccttaccactgagaactccctcacacgtgccgggaaggatgtgcaggctcttctatcacactcttcccctcagctctgcccttctgtctcctggtctcctttctctgttcaaagaggaatgggaagggggcagattcgcagggctatggacatgagtttatgtccaagcagggcatgagatgctggcaccttcttgcaggcaccccctgtctttgtgagtttctctggcatctcctcacttcgttggcagggttgggaacagaggaaaagtcacagcaccacaactcacttccctaggctgtccacttctttttattttcttctttattggaaacatggcttcactctgtctctccgactggagtacagtggcacaaacacagctcactgcagccttccccgctcaggcgatcctcccaccttagcctcccaagtagctgggactacaggtgtgtgccaccatacccggctaatttttgtattttttgtagagacagggcctcgccatattgtccaggctggtctcaaactcctggactcaagcaattctcccatcttggcctcccaaaatgctgggatgacaggcatgagccactgtgcctggccaggctgacaatccctaactcccatgagtccctccctgcaagtgttcagcttaatcagggtcctggggtgatggtcagggtaacagagactccttatgagcctgggagaggctcacttaagattctagagcattttccacctcttttttccccagctttggctcctgggtgccaattctggggtaacaaaaaattttctccttaaaaaaatttctcccattgctattcctgatgatggtgggagcccttgctgatggtgacagtgagaaatggagtattgctgagtattgttcaggaacccagcaggagggtgagggtctcaccccaccccactcatcatgtgcacccacacacactcaaaaattcacattactttggtaaagattaagtttgcttgtgagtgatggaaaacttcaaataacagtgatttaagcaagatagatgttgatttctctctcatgtaacagtctacacaggcaggctagagctggtacagcagttctgccttcctcaggcacccaggtgctttttatctgatttttccaccgcctcacgggacatagtttccacctcatgatctgaaatggctgcttgtgctccagccatcgttcacattccacccaacagaaaggatgaggggacgacaaggggcacacccctgccctttaaggcagctttcttgaagtgtcacatgctgctttaccttacatctgattggccagatgttggtcacatgaccatatctagttgtaagggaaggtgggaaatgtagtcttttgtttgggtggccatgtgcccagttactatcccaatgagaccatctcagctgctcgcatatatggtttgaccatctctctctccctttccccctccttctgccctggtcttgtgggcaatgggcactggcctggggggcaactgcacaggctgtatcatctgctcagaggagaacggctgttccacctgccagcagaggctcttcctgttcatccgccgggaaggcatccgccagtacggcaagtgcctgcacgactgtccccctgggtacttcggcatccgcggccaggaggtcaacaggtgcaaaagtacgtggcttctcccttgttctatgctagtgctgggctcctagacaccatgggcttagatcccaccctttcaccccagcacagacagaggggaagtaggtgcatgtctaagcccagactctgagatacctgcctgtgtccaggctgactccgcagcccgaggcaagtcagtccgctctcagccttaattagcactaatagaacaggcaactttcacttctaccctagtttgtggtgtggccttaggcatgtaactcaccttctctggtcttagctcaaataggaaaaggagtcctaattccaagctctaatttttgctgtaactttgggcaaattatcactatttctgaggtgaaaaagaagcagggagtcctcactccagccctggtttgcgggtgacttcaagcaagtcactcgctctctctgagcctcccctcaaatgagaaagggagtgttctcttcacatgcagcccagtttgaccttggccaagtctgggcttcaaaggagacactgagtcctgacccaaatgctaccaagcctggagctacccagagggccttccacctcgggcagacccagtgggccccatccttggcagtctccctcacctggggtgtccctggctctattacagaatgtggggccacttgtgagagctgcttcagccaggacttctgcatccggtgcaagaggcagttttacttgtacaaggggaagtgtctgcccacctgcccgccgggcactttggcccaccagaacacacgggagtgccagggtgagtggggacctccccgccctgcccctgcccctcccctctccctggagcgggggcttggtgagagatgttaggggccctggaagaaattacagtagaatgccatatggtgagggaaggcccagcaccaccatgtcaggtacactgggtacctccacatagtaactgcaaaacactagagccaacataaccgccattactatgattactactacaaccaacactggcattgttattaatgccaagggaatcagcagagtgcagaagaagagcgctgatgtttatttgtttgtttgtttgcttgtttgcttgttttgtttgagatagggtctcactctgttgcccagctggagtgcagtggcatgatcatagctcactgcagcctcaaactcctaggctcagagaatcctcccgcctcagcctcccgagtaactgggaccacaggcatgtaccacaacagctggctatgcgtttatgtttttgtaaagcctgtgtcctggtcctgagggcttcccaggcaaccttccagcctcctctcctgtccccgccttcctgagacttgactcccaactcaggctgccccatctcccaaacatggtgggccgggctcccaaggctttgcacacctgcctggaatgggcttccttcttctcacctgacgagctcccctctctccctttaactcatacatcacctcctccaggaagcctttcctttaaagctagacagtcggccgctctggcttcaggcccatattagtctgtgcatgcattcgtttcgctctcattccaggctcttataaataactctgctcctagcaggtggaagtggtgtcttcagtcttcagaggactgaacccctcaaaccctgcccttggatctgaaggaccctcactgccggctgaccctgtctgtctgtctgtctgtctctcctttcaccccacaggggagtgtgaactgggtccctggggcggctggagcccctgcacacacaatggaaagacctgcggctcggcttggggcctggagagccgggtacgagaggctggccgggctgggcatgaggaggcagccacctgccaggtgctttctgagtcaaggaaatgtcccatccagaggccctgcccaggaggtgagccccaggacaggcacacgaggctgcggtgggaaaggcccacagggacagggcggactcagatcactgccccacaaatagtatctatgagactgcctgaaaggccaccattagccatactatcatgtggagtacacatcaccttccctggtgtcctttcaaaggagggccttccttgctgggttcagtcccaaatacattaggaaaattgagctggccaggagttcccaaccctctcaaactgaacaccccccttttaataagaaggatttgggaataccctctttatcattcttaatgaagttcatggagaatgtggcctacttgcaaaggtaatttttttttatcagtatagtgtcctaactgtaatatagaggagaaagaaaagaaatttcttgatggaagcgcttggcccaacataccaacagacacttagggaagtacaatcccagtgaggaccgcggccgtggtcagcgccccaagtacgtggccacatcgccatcagagatgcgatttcccaacagtgaccaatgattagtaaggtccaacccaattgatctctgattgacaccacactcacagtgccctagaatctgtgagtttcgtatacataaagcacttggggctgtggcctgcatacagtgagcgcttgctaaatgctgaagtattgttgccacagtgttatgcagaagttggtgcagggacacagatgaaaggtgtccagcgtccagcacagagcctgctcagctatggaaggagtgtgccggggaaagccatggggactcccatgaggccacccgacatgctgattggggggccccaggtgaacctgcaggcctggccgagccagatgtccagcacaagaaggccctgaacaagttagtggccctcgccactccctgaagacctagagagaaaggttcagtttggggtaccttagcccacggtccaaactctcaacaggagggactgcaaggtcagtgcccaaatgttctgagccccgttgttggggagtgggtggggcacccttgtctttcaggactgaggaggctcccaggacctaactggccctgcagccttggtcaccgggctctgtcctctcattgcagagaggagccccggccagaagaagggcaggaaggaccggcgcccacgcaaggacaggaagctggaccgcaggctggacgtgaggccgcgccagcccggcctgcagccctgaccgccggctctcccgactctctggtcctagtcctcggcccctgcacacctcctcctgctccttctcctcctctcctcttactctttctcctctgtcttctccatttgtcctctctttctttccacccttctatcatttttctgtcagtctaccttccctttctttttcttttttatttcctttatttcttccacctccattctcctctcctttctccctccctccttcccttccttcctcttctttctcacttatcttttatctttccttttctttcttcctgtgtttcttcctgtccttcaccgcatccttctctctctccctcctcttgtctccctctcacacacactttaagagggaccatgagcctgtgccctcccctgcagctttctctatctacaacttaaagaaagcaaacatcttttcccaggcctttccctgaccccatctttgcagagaaagggtttccagagggcaaagctgggacacagcacaggtgaatcctgaaggccctgcttctgctctgggggaggctccaggaccctgagctgtgagcacctggttctctggacagtccccagaggccatttccacagccttcagccaccagccaccccgaggagctggctggacaaggctccagggcttccagaggcctggcttggacacctcccccagctggccgtggagggtcacaacctggcctctgggtgggcagccagccctggagggcatcctctgcaagctgcctgccaccctcatcggcactcccccacaggcctccctctcatgggttccatgcccctttttcccaagccggatcaggtgagctgtcactgctgggggatccacctgcccagcccagaagaggccactgaaacggaaaggaaagctgagattatccagcagctctgttccccacctcagcgcttcctgcccatgtggggaaacaggtctgagaaggaaggggcttgcccagggtcacacaggaagccttcaggctctgcttctgcctgatggctctgctcagcacattcacggtggagaggagaatttgggggtcacttgaggggggaaatgtagggaattgtgggtggggagcaagggaagatccgtgcactcgtccacacccaccaccacactcgctgacacccacccccacacgctgacacccacccccacacttgcccacacccatcaccgcactcgcccacacccaccaccacactgccccacacccaccaccacactcccccacacccaccaccacactcgcccacacccaccaccagtgacttgagcatctgtgcttcgctgtgacgcccctcgccctaggcaggaacgacgctgggaggagtctccaggtcagacccagcttggaagcaagtctgtcctcactgcctatccttctgccatcataacacccccttcctgctctgctccccggaatcctcagaaacgggatttgtatttgccgtgactggttggcctgaacacgtagggctccgtgactgggacaggaatgggcaggagaagcaagagtcggagctccaaggggcccaggggtggcctggggaaggaagatggtcagcaggctgggggagaggctctaggtgatgaaatattacattcccgaccccaagagagcacccaccctcagacctgccctccacctggcagctggggagccctggcctgaacccccccctcccagcaggcccaccctctctctgacttccctgctctcacctccccgagaacagctagagccccctcctccgcctggccaggccaccagcttctcttctgcaaacgtttgtgcctctgaaatgctccgttgttattgtttcaagaccctaacttttttttaaaactttcttaataaagggaaaagaaacttgtaaatgcttcttgagcatcaagagggtgttgcaaaaccatgatactgctgagtttggagtagcagaatttaaaacatgtggagtggttttcacaggaatgcttggggctgagaggggtcagagtgtattggggattggggtggggtttcagcttggggggagctgataaaagaggaggggccctcagcccctccaggctactctcaagaagcagactcagccagaggcagaagagggtgacacctcgatccccagaacctcgcagtttcacgaaccagatgtctcagggaccaggggtacctaggaggttgacagtcccacggggccatctaaacaccctgggctgctggtgagagtggccttggcattgggaggcacaggtgggagctccagcctgtcaccagctatctgatggggtccaggtcaagtcacttccccttccggggcctc

SEQ ID NO: 22 - protein, 27 aa, Homo sapiens
MRAPLCLLLLVAHAVDMLALNRRKKQV

SEQ ID NO: 23 - protein, 63 aa, Homo sapiens
GTGLGGNCTGCIICSEENGCSTCQQRLFLFIRREGIRQYGKCLHDCPPGYFGIRGQEVNRCKK

SEQ ID NO: 24 - protein, 47 aa, Homo sapiens
CGATCESCFSQDFCIRCKRQFYLYKGKCLPTCPPGTLAHQNTRECQG

SEQ ID NO: 25 - protein, 62 aa, Homo sapiens
ECELGPWGGWSPCTHNGKTCGSAWGLESRVREAGRAGHEEAATCQVLSESRKCPIQRPCPGE

SEQ ID NO: 26 - protein, 35 aa, Homo sapiens
RSPGQKKGRKDRRPRKDRKLDRRLDVRPRQPGLQP

SEQ ID NO: 27 - DNA, 81 bp, Homo sapiens
atgcgggcgccactctgcctgctcctgctcgtcgcccacgccgtggacatgctcgccctgaaccgaaggaagaagcaagtg

SEQ ID NO: 28 - DNA, 189 bp, Homo sapiens
ggcactggcctggggggcaactgcacaggctgtatcatctgctcagaggagaacggctgttccacctgccagcagaggctcttcctgttcatccgccgggaaggcatccgccagtacggcaagtgcctgcacgactgtccccctgggtacttcggcatccgcggccaggaggtcaacaggtgcaaaaag

SEQ ID NO: 29 - DNA, 141 bp, Homo sapiens
tgtggggccacttgtgagagctgcttcagccaggacttctgcatccggtgcaagaggcagttttacttgtacaaggggaagtgtctgcccacctgcccgccgggcactttggcccaccagaacacacgggagtgccagggg

SEQ ID NO: 30 - DNA, 186 bp, Homo sapiens
gagtgtgaactgggtccctggggcggctggagcccctgcacacacaatggaaagacctgcggctcggcttggggcctggagagccgggtacgagaggctggccgggctgggcatgaggaggcagccacctgccaggtgctttctgagtcaaggaaatgtcccatccagaggccctgcccaggagag

SEQ ID NO: 31 - DNA, 105 bp, Homo sapiens
aggagccccggccagaagaagggcaggaaggaccggcgcccacgcaaggacaggaagctggaccgcaggctggacgtgaggccgcgccagcccggcctgcagccc

SEQ ID NO: 32 - DNA, 20 bp, Artificial Sequence
  Description of Artificial Sequence Synthetic primer
cgtggctacatggtgtattg

SEQ ID NO: 33 - DNA, 20 bp, Artificial Sequence
  Description of Artificial Sequence Synthetic primer
gagtgaggaccttctaggaa

SEQ ID NO: 34 - DNA, 20 bp, Artificial Sequence
  Description of Artificial Sequence Synthetic primer
ggacagggcagtggtttcat

SEQ ID NO: 35 - DNA, 20 bp, Artificial Sequence
  Description of Artificial Sequence Synthetic primer
ggcaacagagcaagattctg

SEQ ID NO: 36 - DNA, 20 bp, Artificial Sequence
  Description of Artificial Sequence Synthetic primer
accttgggcaacatagcaag

SEQ ID NO: 37 - DNA, 20 bp, Artificial Sequence
  Description of Artificial Sequence Synthetic primer
ccggagtttattctccagtg

SEQ ID NO: 38 - DNA, 20 bp, Artificial Sequence
  Description of Artificial Sequence Synthetic primer
ttacaggtgtgagccaccat

SEQ ID NO: 39 - DNA, 20 bp, Artificial Sequence
  Description of Artificial Sequence Synthetic primer
attgcaccactgcactccaa

SEQ ID NO: 40 - DNA, 24 bp, Artificial Sequence
  Description of Artificial Sequence Synthetic primer
cagccgtggtattcagagcaagta

SEQ ID NO: 41 - DNA, 24 bp, Artificial Sequence
  Description of Artificial Sequence Synthetic primer
ggatagtccaggcaagacgtaatg

SEQ ID NO: 42 - DNA, 24 bp, Artificial Sequence
  Description of Artificial Sequence Synthetic primer
ctgggagagtggaaatgggtaagt

SEQ ID NO: 43 - DNA, 24 bp, Artificial Sequence
  Description of Artificial Sequence Synthetic primer
cacaggacgttccaccacacttga

SEQ ID NO: 44 - DNA, 24 bp, Artificial Sequence
  Description of Artificial Sequence Synthetic primer
caacccagaacctggcacaaagca

SEQ ID NO: 45 - DNA, 24 bp, Artificial Sequence
  Description of Artificial Sequence Synthetic primer
ggtctgtctgcttagccacatttg

SEQ ID NO: 46 - DNA, 24 bp, Artificial Sequence
  Description of Artificial Sequence Synthetic primer
gccttagtcttttccctctagcag

SEQ ID NO: 47 - DNA, 24 bp, Artificial Sequence
  Description of Artificial Sequence Synthetic primer
gacaaagccactggggaagttctt

SEQ ID NO: 48 - DNA, 24 bp, Artificial Sequence
  Description of Artificial Sequence Synthetic primer
gcaacagccccgattagtctttgt

SEQ ID NO: 49 - DNA, 20 bp, Artificial Sequence
  Description of Artificial Sequence Synthetic primer
ccaacgccctcactagacct

SEQ ID NO: 50 - DNA, 24 bp, Artificial Sequence
  Description of Artificial Sequence Synthetic primer
ccatctcagctgctcgcatatatg

SEQ ID NO: 51 - DNA, 24 bp, Artificial Sequence
  Description of Artificial Sequence Synthetic primer
cactgagtcctgacccaaatgcta

SEQ ID NO: 52 - DNA, 24 bp, Artificial Sequence
  Description of Artificial Sequence Synthetic primer
tcaaaccctgcccttggatctgaa

SEQ ID NO: 53 - DNA, 24 bp, Artificial Sequence
  Description of Artificial Sequence Synthetic primer
ggcacccttgtctttcaggactga

SEQ ID NO: 54 - DNA, 24 bp, Artificial Sequence
  Description of Artificial Sequence Synthetic primer
ctgcagccaccagccaagttcttt

SEQ ID NO: 55 - DNA, 24 bp, Artificial Sequence
  Description of Artificial Sequence Synthetic primer
cgtctccattttggtctcaggtgt

SEQ ID NO: 56 - DNA, 24 bp, Artificial Sequence
  Description of Artificial Sequence Synthetic primer
catctgtgaagtgaggtgggtaag

SEQ ID NO: 57 - DNA, 24 bp, Artificial Sequence
  Description of Artificial Sequence Synthetic primer
gtctccaagccttaggaaggtatt

SEQ ID NO: 58 - DNA, 24 bp, Artificial Sequence
  Description of Artificial Sequence Synthetic primer
ggtgaggacagaggagtagccaat

SEQ ID NO: 59 - DNA, 24 bp, Artificial Sequence
  Description of Artificial Sequence Synthetic primer
cccttgtgctatggtctcatgcaa

SEQ ID NO: 60 - DNA, 24 bp, Artificial Sequence
  Description of Artificial Sequence Synthetic primer
gccttttctccaagggcagtcctt

SEQ ID NO: 61 - DNA, 24 bp, Artificial Sequence
  Description of Artificial Sequence Synthetic primer
cctcacaccgccacatcatgttga

SEQ ID NO: 62 - DNA, 24 bp, Artificial Sequence
  Description of Artificial Sequence Synthetic primer
cctgactcaccttcatgtgcttag

SEQ ID NO: 63 - DNA, 24 bp, Artificial Sequence
  Description of Artificial Sequence Synthetic primer
ggatggacactccacctgctgatt

SEQ ID NO: 64 - DNA, 24 bp, Artificial Sequence
  Description of Artificial Sequence Synthetic primer
accctactcagggtcagagatcaa

SEQ ID NO: 65 - DNA, 24 bp, Artificial Sequence
  Description of Artificial Sequence Synthetic primer
ccctaggtcctaaacttgactcca

SEQ ID NO: 66 - DNA, 24 bp, Artificial Sequence
  Description of Artificial Sequence Synthetic primer
gcctggattgggattgttgttgac

SEQ ID NO: 67 - DNA, 24 bp, Artificial Sequence
  Description of Artificial Sequence Synthetic primer
cccctacacctctggtatttcaga

SEQ ID NO: 68 - DNA, 24 bp, Artificial Sequence
  Description of Artificial Sequence Synthetic primer
gcagatgtgcactgtcagctttag

SEQ ID NO: 69 - DNA, 24 bp, Artificial Sequence
  Description of Artificial Sequence Synthetic primer
gaccgttggagcagactctgtaga

SEQ ID NO: 70 - DNA, 24 bp, Artificial Sequence
  Description of Artificial Sequence Synthetic primer
tggaggggacttcaaggactcaat

SEQ ID NO: 71 - DNA, 24 bp, Artificial Sequence
  Description of Artificial Sequence Synthetic primer
ggcagcttccgatgtgcaaataca

SEQ ID NO: 72 - DNA, 24 bp, Artificial Sequence
  Description of Artificial Sequence Synthetic primer
gttgagactcgtctggaggagcga

SEQ ID NO: 73 - DNA, 24 bp, Artificial Sequence
  Description of Artificial Sequence Synthetic primer
ctgggcttagacatgcacctactt

SEQ ID NO: 74 - DNA, 24 bp, Artificial Sequence
  Description of Artificial Sequence Synthetic primer
ccctcaccatatggcattctactg

SEQ ID NO: 75 - DNA, 24 bp, Artificial Sequence
  Description of Artificial Sequence Synthetic primer
cctttcaggcagtctcatagatac

SEQ ID NO: 76 - DNA, 21 bp, Artificial Sequence
  Description of Artificial Sequence Synthetic primer
cgaggactaggaccagagagt

SEQ ID NO: 77 - DNA, 24 bp, Artificial Sequence
  Description of Artificial Sequence Synthetic primer
cccctgatccatccagcactttct

SEQ ID NO: 78 - DNA, 24 bp, Artificial Sequence
  Description of Artificial Sequence Synthetic primer
ggtactctgagtttgggcagaaga

SEQ ID NO: 79 - DNA, 24 bp, Artificial Sequence
  Description of Artificial Sequence Synthetic primer
gaagctctggattcgtaccgttaa

SEQ ID NO: 80 - DNA, 24 bp, Artificial Sequence
  Description of Artificial Sequence Synthetic primer
ggacacatccaccctattcctcat

SEQ ID NO: 81 - DNA, 24 bp, Artificial Sequence
  Description of Artificial Sequence Synthetic primer
ggatagctggctggaatccctcta

SEQ ID NO: 82 - DNA, 24 bp, Artificial Sequence
  Description of Artificial Sequence Synthetic primer
ggctgaaaagccacaaaagcaagt

SEQ ID NO: 83 - DNA, 24 bp, Artificial Sequence
  Description of Artificial Sequence Synthetic primer
accttcattgctgccacactgaac

SEQ ID NO: 84 - DNA, 24 bp, Artificial Sequence
  Description of Artificial Sequence Synthetic primer
gtctgcaaacacatgagcagaatc

SEQ ID NO: 85 - DNA, 24 bp, Artificial Sequence
  Description of Artificial Sequence Synthetic primer
agcctggtgctctatcgtgctctt

SEQ ID NO: 86 - DNA, 24 bp, Artificial Sequence
  Description of Artificial Sequence Synthetic primer
tggaccttgtagcactggagctaa

SEQ ID NO: 87 - DNA, 24 bp, Artificial Sequence
  Description of Artificial Sequence Synthetic primer
cagcagaggctcttcctcttcatc

SEQ ID NO: 88 - DNA, 24 bp, Artificial Sequence
  Description of Artificial Sequence Synthetic primer
gagccacaggtcttcccattgtgt

SEQ ID NO: 89 - DNA, 20 bp, Artificial Sequence
  Description of Artificial Sequence Synthetic primer
ccctgtggtgttagattgga

SEQ ID NO: 90 - DNA, 20 bp, Artificial Sequence
  Description of Artificial Sequence Synthetic primer
caccccagcaggcattgatt

SEQ ID NO: 91 - DNA, 20 bp, Artificial Sequence
  Description of Artificial Sequence Synthetic primer
ctcacacagccttcatgaag

SEQ ID NO: 92 - DNA, 20 bp, Artificial Sequence
  Description of Artificial Sequence Synthetic primer
cttctggtactttcctccat

SEQ ID NO: 93 - DNA, 20 bp, Artificial Sequence
  Description of Artificial Sequence Synthetic primer
gacctaccactgatcttgtt

SEQ ID NO: 94 - DNA, 20 bp, Artificial Sequence
  Description of Artificial Sequence Synthetic primer
gcactgaggctctttgagtt

SEQ ID NO: 95 - DNA, 20 bp, Artificial Sequence
  Description of Artificial Sequence Synthetic primer
tctggaccatgcctgtcttt

SEQ ID NO: 96 - DNA, 20 bp, Artificial Sequence
  Description of Artificial Sequence Synthetic primer
gccacttctttgagtcttca

SEQ ID NO: 97 - DNA, 31 bp, Homo sapiens
tggcctggggggcaactgcacaggctgtatc

SEQ ID NO: 98 - DNA, 31 bp, Homo sapiens
tggcctggctgtatcatctgctcagaggaga

SEQ ID NO: 99 - DNA, 21 bp, Homo sapiens
ggcatccgccagtacggcaag

SEQ ID NO: 100 - DNA, 21 bp, Homo sapiens
ggcatccgccggtacggcaag

SEQ ID NO: 101 - protein, 110 aa, Homo sapiens
GTGLGGNCTGCIICSEENGCSTCQQRLFLFIRREGIRRYGKCLHDCPPGYFGIRGQEVNRCKKCGATFESCFSQDFCIRRKRQFYLYKGKYLPTCPPGTLAHQNTRECQG

SEQ ID NO: 102 - protein, 110 aa, Homo sapiens
GTGLGGNCTGCIICSEENGCSTCQQRLFLFIRREGIRQYGKCLHDCPPGYFGIRGQEVNRCKKCGATCESCFSQDFCIRCKRQFYLYKGKCLPTCPPGTLAHQNTRECQG

SEQ ID NO: 103 - protein, 114 aa, Homo sapiens
SAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPA

SEQ ID NO: 104 - protein, 111 aa, Homo sapiens
SYVSNPICKGCLSCSKDNGCSRCQQKLFFFLRREGMRQYGECLHSCPSGYYGHRAPDMNRCARCRIENCDSCFSKDFCTKCKVGFYLHRGRCFDECPDGFAPLEETMECVE

SEQ ID NO: 105 - protein, 113 aa, Homo sapiens
HPNVSQGCQGGCATCSDYNGCLSCKPRLFFALERIGMKQIGVCLSSCPSGYYGTRYPDINKCTKCKADCDTCFNKNFCTKCKSGFYLHLGKCLDNCPEGLEANNHTMECVSIV

SEQ ID NO: 106 - DNA, 21 bp, Homo sapiens
ctctattacagaatgtggggc

SEQ ID NO: 107 - DNA, 21 bp, Homo sapiens
ctctattacaaaatgtggggc

SEQ ID NO: 108 - DNA, 21 bp, Homo sapiens
  modified_base (11)..(11): a, c, t, g, unknown or other
ctctattacanaatgtggggc

SEQ ID NO: 109 - DNA, 38 bp, Homo sapiens
gatccggctgcccagatgcgggcgccactctgcctgct

SEQ ID NO: 110 - DNA, 26 bp, Homo sapiens
gctgcccagatgcgggcgccactctg

SEQ ID NO: 111 - DNA, 38 bp, Homo sapiens
gatccgcctgctcctgctcgtcgcccacgccgtggaca

SEQ ID NO: 112 - DNA, 19 bp, Homo sapiens
  modified_base (7)..(19): a, c, t, g, unknown or other
gatccgnnnnnnnnnnnnn

SEQ ID NO: 113 - DNA, 19 bp, Homo sapiens
tgcccagatgcgggcgcca

SEQ ID NO: 114 - DNA, 19 bp, Homo sapiens
tgcccagatacgggcgcca

SEQ ID NO: 115 - DNA, 19 bp, Homo sapiens
  modified_base (10)..(10): a, c, t, g, unknown or other
tgcccagatncgggcgcca

SEQ ID NO: 116 - DNA, 26 bp, Homo sapiens
gctgcccagatgcgggcgccactctg

(19) United States
(12) Patent Application Publication
(10) Pub. No.: US 2009/0208484 A1
(43) Pub. Date: Aug. 20, 2009

(54) R-SPONDIN COMPOSITIONS AND METHODS OF USE THEREOF

(75) Inventor: Angela M. Christiano, Upper Saddle River, NJ (US)

Correspondence Address: WilmerHale/Columbia University, 399 PARK AVENUE, NEW YORK, NY, 10022, US

(73) Assignee: THE TRUSTEES OF COLUMBIA UNIVERSITY IN THE CITY OF NEW YORK, New York, NY (US)

(21) Appl. No.: 12/369,893

(22) Filed: Feb. 12, 2009

Related U.S. Application Data
(63) Continuation of application No. PCT/US2007/016197, filed on Jul. 17, 2007.
(60) Provisional application No. 60/837,546, filed on Aug. 14, 2006.

(30) Foreign Application Priority Data
Jan. 16, 2007 (JP) 2007-007227

(51) Int. Cl. A61K 39/395; A61K 31/7088; A61K 31/7105; A61K 38/00; C07K 14/435; C07H 21/00; C12Q 1/68; C12Q 1/02; C40B 30/00

(52) U.S. Cl. 424/130.1; 514/15; 514/17; 530/350; 536/23.5; 435/6; 435/29; 506/7; 514/44 R

(57) ABSTRACT

The invention provides for a method for screening compounds that bind to and modulate a regulator of Wnt signaling, R-spondin 4. The invention further provides for methods for diagnosing a keratin-related abnormality, such as anonychia congenital, in a subject. The invention also provides for isolated RSPO4 mutant molecules.

This application is a continuation of International Patent Application No. PCT/IS2007/016197, filed Jul. 17, 2007, which claims the benefit of U.S. Provisional Patent Application No. 60/837,546, filed Aug. 14, 2006, and Japanese Patent Application No. 2007-007227, filed Jan. 16, 2007. All patents, patent applications and publications cited herein are hereby incorporated by reference in their entirety. The disclosures of these publications in their entireties are hereby incorporated by reference into this application.

### GOVERNMENT INTERESTS

The work described herein was supported in whole, or in part, by National Institute of Health Grant No. NIH RO1 AR44924. Thus, the United States Government has certain rights to the invention.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND

Anonychia/hyponychia congenita (OMIM 206800) is a rare, usually autosomal recessive condition. Most cases of anonychia occur as part of syndromes, particularly in association with hypoplasia or absence of distal phalanges, for example, Cooks syndrome (OMIM 106995). In isolated (non-syndromic) anonychia, there is variable expression of the nail phenotypes ranging from individuals with no nail field at all to a nail field of reduced size with an absent or diminutive nail rudiment. The nail plate (visible part of the nail) is a keratinized structure that grows continuously due to maturation and keratinization of the nail matrix (a germinative epithelium located beneath the cuticle). The nail plate is closely attached to the nail bed (the skin beneath the nail plate), and the dermis of the nail bed is attached to the distal phalanx of the digit. Hence, malformations of the nail are frequently found in combination with underlying bone alterations. The nail plate separates the tissue beneath the nail (subungual) and beside it (periungual), thereby maintaining the dimensions of the nail field. When the nail plate is absent or malformed, as in anonychia, the nail field is subsequently reduced in size.

### SUMMARY OF THE INVENTION

An aspect of the present invention is directed to a method for treating a nail, hoof, or claw keratin-related abnormality in a subject, wherein the method comprises administering to the subject an effective amount of a composition comprising a R-spondin 4 modulating compound, thereby treating keratin-related abnormality in the subject. In one embodiment, the abnormality is characterized by weakening of the nail, hoof, or claw. In another embodiment, the abnormality is characterized by slow or absent growth or repair of the nail, hoof, or claw. In a further embodiment, the abnormality is characterized by hyperplasia of the nail, hoof, or claw. In some embodiments, the abnormality is an inherited abnormality. In particular embodiments, the inherited abnormality is selected from the group consisting of anonychia congenita, hyponychia congenita, Cooks syndrome, nail patella syndrome, ectodermal dysplasias, and epidermolysis bullosa. In further embodiments, the abnormality is caused by an infection of the nail, hoof, or claw. In other embodiments, infection is caused by a bacterium, a fungus, a yeast, a mold, a virus, or any combination thereof. In one embodiment, the compound comprises an antibody directed to R-spondin 4 comprising SEQ ID NO: 1, or a fragment thereof. In another embodiment, the compound comprises a R-spondin 4 antisense RNA or antisense DNA; a R-spondin 4 siRNA; or a combination of nucleic acids described. In a further embodiment, the compound decreases expression of R-spondin 4 via RNA interference. In other embodiments, R-spondin 4 comprises a polynucleotide molecule comprising SEQ ID NO: 2, 27, 28, 29, 30, or 31. In some embodiments, the compound comprises a R-spondin 4 polypeptide molecule comprising at least 10 amino acids of SEQ ID NO: 1, or a fragment, variant, or peptidomimetic thereof, while in other embodiments, the compound comprises a R-spondin 4 peptide comprising at least 5 amino acids of SEQ ID NO: 22, or a fragment, variant, or peptidomimetic thereof. In further embodiments, the compound comprises a R-spondin 4 peptide comprising at least 5 amino acids of SEQ ID NO: 23, or a fragment, variant, or peptidomimetic thereof. In some embodiments, the compound comprises a R-spondin 4 peptide comprising at least 5 amino acids of SEQ ID NO: 24, or a fragment, variant, or peptidomimetic thereof. In further embodiments, the compound comprises a R-spondin 4 peptide comprising at least 5 amino acids of SEQ ID NO: 25, or a fragment, variant, or peptidomimetic thereof. In other embodiments, the compound comprises a R-spondin 4 peptide comprising at least 5 amino acids of SEQ ID NO: 26, or a fragment, variant, or peptidomimetic thereof. In particular embodiments, the compound comprises a R-spondin 4 peptide having at least 46%, 48%, 50%, 55%, 60%, 70%, 75%, 80%, 90%, 95%, or 99% identity to SEQ ID NO: 1. In yet further embodiments of the invention, the subject is a human, a cat, a dog, a horse, a cow, a sheep, a goat, a pig, a chicken, an avian, a domestic pet, or a mammal reared for agricultural uses. In some embodiments, the composition is administered to, or in the vicinity of, one of more nails, hooves, or claws, while in other embodiments of the invention, the composition is administered topically. In particular embodiments, the composition is formulated as a cream or lotion, an oil, or as a paint or lacquer. In further embodiments, the composition comprises one or more carriers, excipients, solvents or bases.

One aspect of the invention is directed to a method for strengthening, repairing, or stimulating growth of a nail, hoof, or claw in a subject, wherein the method comprises administering to the subject an effective amount of a composition comprising a R-spondin 4 modulating compound, wherein the compound increases the activity or the expression of R-spondin 4. Another aspect of the invention provides for a method for inhibiting the growth of, or weakening, a nail, hoof, or claw in a subject, wherein the method comprises administering to the subject an effective amount of a composition comprising a R-spondin 4 modulating compound, wherein the compound decreases the activity or the expression of R-spondin 4. In one embodiment, the compound comprises an antibody directed to R-spondin 4 comprising SEQ ID NO: 1, or a fragment thereof. In another embodiment, the compound comprises a R-spondin 4 antisense RNA or antisense DNA; a R-spondin 4 siRNA; or a combination of nucleic acids described. In a further embodiment, the compound decreases expression of R-spondin 4 via RNA interference. In other embodiments, R-spondin 4 comprises a polynucleotide molecule comprising SEQ ID NO: 2, 27, 28, 29, 30, or 31. In some embodiments, the compound comprises a R-spondin 4 polypeptide molecule comprising at least 10 amino acids of SEQ ID NO: 1, or a fragment, variant, or peptidomimetic thereof, while in other embodiments, the compound comprises a R-spondin 4 peptide comprising at least 5 amino acids of SEQ ID NO: 22, or a fragment, variant, or peptidomimetic thereof. In further embodiments, the compound comprises a R-spondin 4 peptide comprising at least 5 amino acids of SEQ ID NO: 23, or a fragment, variant, or peptidomimetic thereof. In some embodiments, the compound comprises a R-spondin 4 peptide comprising at least 5 amino acids of SEQ ID NO: 24, or a fragment, variant, or peptidomimetic thereof. In further embodiments, the compound comprises a R-spondin 4 peptide comprising at least 5 amino acids of SEQ ID NO: 25, or a fragment, variant, or peptidomimetic thereof. In other embodiments, the compound comprises a R-spondin 4 peptide comprising at least 5 amino acids of SEQ ID NO: 26, or a fragment, variant, or peptidomimetic thereof. In particular embodiments, the compound comprises a R-spondin 4 peptide having at least 46%, 48%, 50%, 55%, 60%, 70%, 75%, 80%, 90%, 95%, or 99% identity to SEQ ID NO: 1. In yet further embodiments of the invention, the subject is a human, a cat, a dog, a horse, a cow, a sheep, a goat, a pig, a chicken, an avian, a domestic pet, or a mammal reared for agricultural uses. In some embodiments, the composition is administered to, or in the vicinity of, one of more nails, hooves, or claws, while in other embodiments of the invention, the composition is administered topically. In particular embodiments, the composition is formulated as a cream or lotion, an oil, or as a paint or lacquer. In further embodiments, the composition comprises one or more carriers, excipients, solvents or bases.

An aspect of the invention is directed to a method for identifying a compound that modulates R-spondin 4 activity, wherein the method comprises (a) expressing R-spondin 4 in a cell; (b) contacting the cell with a ligand source for an effective period of time; (c) measuring a secondary messenger response; (d) isolating the ligand from the ligand source; and (e) identifying the structure of the ligand that binds R-spondin 4, thereby identifying which compound would modulate the activity of R-spondin 4. In one embodiment, the method can further comprise: (f) obtaining or synthesizing the compound determined to bind to R-spondin 4 or to be a potential modulator of R-spondin 4 activity; (g) contacting R-spondin 4 protein with the compound under a condition suitable for binding; and (h) determining whether the compound modulates R-spondin 4 activity using a diagnostic assay. In another embodiment, the compound is a R-spondin 4 agonist or a R-spondin 4 antagonist. In a further embodiment, the antagonist decreases R-spondin 4 expression or R-spondin 4 activity by at least 10%, 20%, 30%, 40%, 50%, 60%, 70%, 75%, 80%, 90%, 95%, 99%, or 100%. In other embodiments, the agonist increases R-spondin 4 expression or R-spondin 4 activity by at least 10%, 20%, 30%, 40%, 50%, 60%, 70%, 75%, 80%, 90%, 95%, 99%, or 100%. In some embodiments, the compound comprises an antibody directed to R-spondin 4 or a fragment thereof, a R-spondin 4 antisense RNA or antisense DNA; a R-spondin 4 siRNA; or a R-spondin 4 peptide comprising at least 10 amino acids of SEQ ID NO: 1, or a fragment or variant thereof. In other embodiments of the invention, the cell is a bacterium, a yeast, an insect cell, or a mammalian cell. In further embodiments, the ligand source is a compound library. In particular embodiments, measuring comprises detecting an increase or decease in a secondary messenger concentration. In other embodiments, the assay determines the concentration of the secondary messenger within the cell. In some embodiments, the secondary messenger comprises Tcf1, Lef1, phosphorylated Dsh, Axin, β-catenin, or a combination thereof. In other embodiments of the invention, contacting comprises administering the compound to a mammal in vivo or a cell in vitro. In other embodiments, the mammal is a mouse.

One aspect of the invention is directed to an isolated mutant human R-spondin 4 polypeptide comprising a C>Y mutation at amino acid position 118 of SEQ ID NO: 1, comprising the amino acid sequence of SEQ ID NO: 6.

Another aspect of the invention provides for an isolated mutant human R-spondin 4 polypeptide comprising a M>I mutation at amino acid position 1 of SEQ ID NO: 1, comprising the amino acid sequence of SEQ ID NO: 14.

An aspect of the invention is also directed to an isolated mutant human R-spondin 4 polypeptide encoded by a nucleic acid comprising the sequence of SEQ ID NO: 11.

Another aspect of the invention provides for an isolated mutant human R-spondin 4 polynucleotide comprising the nucleic acid sequence of SEQ ID NO: 10, 11, or 15.

An aspect of the invention is directed to a pharmaceutical, veterinary, or cosmetic composition comprising a R-spondin 4 polypeptide molecule having at least 46%, 48%, 50%, 55%, 60%, 70%, 75%, 80%, 90%, 95%, or 99% identity to SEQ ID NO: 1, or a variant, fragment, or peptidomimetic thereof. In one embodiment, R-spondin 4 comprises at least 5 amino acids comprising the amino acid sequence of SEQ ID NO: 22, 23, 24, 25, or 26. In another embodiment, R-spondin comprises at least 10 amino acids comprising the amino acid sequence of SEQ ID NO: 1. In a further embodiment, R-spondin 4 antisense RNA or antisense DNA; a R-spondin 4 siRNA; or a combination thereof. In some embodiments, R-spondin 4 comprises a polynucleotide molecule comprising SEQ ID NO: 2, 27, 28, 29, 30, or 31. In other embodiments, the composition is formulated for administration to, or in the vicinity of, one of more nails, hooves or claws. In further embodiments, the composition is formulated for topical administration. In particular embodiments, the composition is formulated as a cream or lotion, an oil, or a paint or lacquer. In some embodiments of the invention, the composition further comprises one or more carriers, excipients, solvents or bases. In other embodiments, the R-spondin is present in a therapeutically or cosmetically effective amount.

One aspect of the invention provides a method for diagnosing anonychia congenita in a subject. The present invention provides methods for the diagnosis of inherited diseases, disorders, syndromes and the like that affect keratin-containing limb appendages, such as a nails, hooves, or claws. The method comprises testing the subject for a mutation in the R-spondin 4 gene, wherein a DNA sample is obtained from the subject. In one embodiment, the subject is a human. In another embodiment, the mutation comprises a nucleic acid sequence comprising SEQ ID NO: 11, wherein the first 26 nucleic acid residues from SEQ ID NO: 2 are deleted; a nucleic acid sequence comprising SEQ ID NO: 10, wherein a G>A mutation occurs at nucleic acid position +353 of SEQ ID NO: 2; a nucleic acid sequence comprising SEQ ID NO: 15, wherein an G>A mutation occurs at nucleic acid position +3 of SEQ ID NO: 2; or a combination thereof. In a further embodiment, the mutation comprises a nucleic acid encoding a polypeptide molecule comprising an amino acid sequence comprising SEQ ID NO: 6, wherein a C>Y mutation occurs at amino acid position 118 of SEQ ID NO: 1; a nucleic acid encoding a polypeptide molecule comprising an amino acid sequence comprising SEQ ID NO: 14, wherein a M>I mutation occurs at amino acid position 1 of SEQ ID NO: 1; or a combination thereof. In some embodiments, the mutation comprises a nucleic acid comprising SEQ ID NO: 16, wherein a G>A mutation occurs at nucleic acid position 3077 of SEQ ID NO: 19; a nucleic acid comprising SEQ ID NO: 17, wherein a G>A mutation occurs at nucleic acid position 3711 of SEQ ID NO: 19; a nucleic acid comprising SEQ ID NO: 20, wherein a G>A mutation occurs at nucleic acid position 809 of SEQ ID NO: 19; or a combination thereof. In other embodiments, the mutation comprises a G>A nucleic acid mutation at about nucleotide position 3853 of SEQ ID NO: 19, which lies at the intron 3-exon 3 boundary; a G>A nucleic acid mutation at about nucleotide position 4797 of SEQ ID NO: 19, which lies at the intron 3-exon 4 boundary; a G>A nucleic acid mutation at about nucleotide position 4984 of SEQ ID NO: 19, which lies at the intron 4-exon 4 boundary; a G>A nucleic acid mutation at about nucleotide position 6095 of SEQ ID NO: 19, which lies at the intron 4-exon 5 boundary; or a combination thereof. In further embodiments, the mutation occurs in a nucleic acid sequence encoding a polypeptide molecule comprising SEQ ID NO: 22, 23, 24, 25, 26, or a combination thereof. In yet other embodiments of the invention, the mutation attenuates the function of the R-spondin 4 protein or produces a truncated R-spondin protein.

### BRIEF DESCRIPTION OF THE FIGURES

FIG. 1A is a photograph of a hand displaying a clinical phenotype seen in anonychia patients. The nail field is reduced in size, the nail plate is absent, the nail matrix is swollen (see arrow), and the nail bed has protective hyperkeratosis.

FIG. 1B is a photograph of a hand displaying a clinical phenotype seen in anonychia patients. The nail field is reduced in size, the nail plate is absent, and the nail bed has protective hyperkeratosis.

FIG. 1C is a graph depicting a parametric LOD score analysis of genome-wide SNP genotypes with Allegro for three anonychia families (PI, Finnish, and Irish).

FIG. 1D is a diagram of two Pedigrees (PI and F) with genotypes of microsatellite/SNPs mapping to 20p13. Affected individuals are indicated by filled symbols. The red boxes indicate the microsatellite markers used in original genome-wide linkage analysis.

FIG. 2A is a diagram that depicts haplotypes of family P2. Additional microsatellite markers between D20S117 and D20S906 were used to fine map the linkage in family P2.

FIG. 2B. is a schematic showing the minimal region harboring the anonychia gene based on recombination mapping. Additional microsatellite markers between D20S117 and D20S906 were used to fine map the linkage in family P2. The linked haplotype is indicated in red. RSPO4 is one of four genes mapping within the minimal region of linkage, indicated in pink.

FIG. 2C represents DNA chromatographs depicting examples of two mutations (32 GfsX220 mutant, left panel (SEQ ID NOS 97 (top) and 98 (bottom)); Q65R mutant, right panel (SEQ ID NOS 99 (top) and 100 (bottom))) detected in the anonychia families. Normal sequence traces are shown on the top row and the mutant sequences are shown in the bottom row of each panel. The box in the left panel (top row) indicates the 16 bp deletion and the arrow (right panel) indicates the A to G base change.

FIG. 2D is a schematic of the genomic structure of RSPO4 gene showing the positions of all the detected mutations. The family identifier is shown above each mutation (F, Finnish; I, Irish). The RSPO4 mRNA sequence is numbered according to NM_{—}001029871, with nucleotide numbering starting from the first ATG codon. Amino acid substitutions are shown in brackets with reference to amino acid sequence NP_{—}001025042.

FIG. 2E represents an amino acid alignment of the residues affected by the missense mutations showing the conservation among R-spondin paralogs. FIG. 2E discloses SEQ ID NOS 101-105, respectively, in order of appearance.

FIG. 2F is a photograph of whole mount in situ of RSPO4 in mouse embryogenesis (e15.5) showing specific expression at the sites of nail development. The RSPO4 probe is labeled AS (antisense) (left panel, top) and the negative control is labeled S (sense) (right panel, top). A section (bottom panel) reveals RSPO4 expression is confined to the nail mesoderm.

FIG. 3 is a schematic of pedigree P2 with genotype data. Affected individuals are indicated by filled symbols and the linked haplotype on chromosome 20p13 is shown in red. Linkage studies of several anonychia families indicated that the mutation mapped on Chr20p13, a region of 1292 Kbp. Genotyping of additional large anonychia families from Pakistan reduced the region to 850 Kbp (D20 S17-D₂OS906).

FIG. 4 is a diagram depicting a spectrum of RSPO4 mutations in inherited Anonychia.

FIG. 5A is a photograph of whole mount in situ of RSPO4 in mouse embryogenesis (e15.5). Rspo4 mRNA is expressed at sites of nail development, at the tips of fingers (Left Panel) and around vibrissa (Right Panel).

FIG. 5B is a photograph of whole mount in situ of RSPO4 in mouse embryogenesis (e15.5). Rspo4 mRNA is expressed at the tips of fingers and toes, and around vibrissa (Left Panel). The negative control shows no mRNA expression (Right Panel).

FIG. 6 is a photograph of a section through the nail region showing that Rspo4 mRNA expression is confined to the nail mesoderm. The boundary between dermis and epidermis is marked with a broken line, and the green asterisk denotes the tip of the finger.

FIG. 7 is a schematic of the role R-spondin plays in signaling.

FIG. 8A-C are diagrams of pedigrees representing Pakistani families N1-N3 with anonychia. Affected males and females are indicated by filled squares and circles, respectively. Double lines between figures are representative of consanguineous unions. DNA was obtained from numbered individuals in this study.

FIG. 8D-E are diagrams of pedigrees representing Pakistani families N4-N5 with anonychia. Affected males and females are indicated by filled squares and circles, respectively. Double lines between figures are representative of consanguineous unions. DNA was obtained from numbered individuals in this study.

FIG. 8F-G are photographs of the clinical features of inherited anonychia in the fingernails (FIG. 8F) and toenails (FIG. 8G).

FIG. 9A depicts homozygous mutations in RSPO4 that are responsible for anonychia. Families N1, N2 and N4 have a IVS-1G>A mutation at the exon 2-intron 2 boundary. A diagram of a pedigree (Top) is shown that represents a Pakistani family N2 with inherited anonychia. DNA chromatograms illustrate the wild type (SEQ ID NO: 106) (top chromatogram), homozygous (SEQ ID NO: 107) (middle chromatogram), and heterozygous (SEQ ID NO: 108) (bottom chromatogram) mutants.

FIG. 9B depicts homozygous mutations in RSPO4 that are responsible for anonychia. Family N3 has a −9-+17del26 mutation in exon 1 (26 bp deletion disclosed as SEQ ID NO: 110). A diagram of a pedigree (Top) is shown that represents a Pakistani family N3 with inherited anonychia. DNA chromatograms illustrate the wild type (SEQ ID NO: 109) (top chromatogram), homozygous (SEQ ID NO: 111) (middle chromatogram), and heterozygous (SEQ ID NO: 112) (bottom chromatogram) mutants.

FIG. 9C depicts homozygous mutations in RSPO4 that are responsible for anonychia. Family N5 has a 3G>A (M1I) mutation in exon 1. A diagram of a pedigree (Top) is shown that represents a Pakistani family N5 with inherited anonychia. DNA chromatograms illustrate the wild type (SEQ ID NO: 113) (top chromatogram), homozygous (SEQ ID NO: 114) (middle chromatogram), and heterozygous (SEQ ID NO: 115) (bottom chromatogram) mutants

FIG. 9D is a Schematic of reported RSPO4 gene mutations. −9-+17del26: Pakistani P2 (see Example 1), Pakistani N3 (see Example 2); 3G>A: Pakistani N5 (see Example 2); IVS1+1G>A: Pakistani P4, Irish (see Example 1); IVS-1G>A: English E1 (see Example 1); 92_{—}93insG: German (Bergmann et al., 2006 Am J Hum Genet. 79, 1105-1109); 95-110del16: Indian In1 (see Example 1); 194A>G: Finnish (see Example 1); 218G>A: German (Bergmann et al., 2006 Am J Hum Genet. 79, 1105-1109); IVS2-1G>A: Pakistani N1, N2, N4 (see Example 2); 284G>T: English E1, E2 (see Example 1); 319T>C: Irish, English E2 (see Example 1); 353G>A: Pakistani P3 (see Example 1).

FIG. 10A-B are reproductions of in situ hybridization studies showing mRspo3 (FIG. 10A) or mRspo4 (FIG. 10B) being expressed in the nail field mesenchyme at e14.5.

FIG. 10C is a reproduction of a northern blot depicting mRspo4 expression in e14.5 dermis and in adult whole skin. mRspo4 is not present in e14.5 epidermis, as detected by RT-PCR. D, dermis; E, epidermis; WS, whole skin.

### DETAILED DESCRIPTION

Very little is known about the molecular signals involved in the development of the nail. Formation of the human set of nails begins in the 9^{th} week of gestation and is completed by week 20, with development of the toenails lagging approximately four weeks behind the fingernails. Much of what is known about the genes involved in this process has been inferred from the study of mouse models as well as human genetic disorders in which nail dysplasia forms part of the phenotype.

Proper epithelial-mesenchymal interactions both within the skin, as well as the underlying bone appear crucial for nail development and growth factors such as bone morphogenic protein-4 and fibroblast growth factor-4, as well as signaling molecules such as Wnt7A and sonic hedgehog all play an important role (Chuong, C. M., Widelitz, R. B., Ting-Berreth, S. & Jiang, T. X. Early events during avian skin appendage regeneration: dependence on epithelial-mesenchymal interaction and order of molecular reappearance. J Invest Dermatol 107, 639-46 (1996)). Expression of transcription factors such as LMY1B and MSX1 are also essential, as seen when they are mutated in nail-patella-syndrome (NPS, OMIM 256020) and Witkop syndrome (OMIM 189500), respectively (Dreyer, S. D. et al. Mutations in LMX1B cause abnormal skeletal patterning and renal dysplasia in nail patella syndrome. Nat Genet. 19, 47-50 (1998); Chen, H. et al. Limb and kidney defects in Lmx1b mutant mice suggest an involvement of LMX1B in human nail patella syndrome. Nat Genet. 19, 51-5 (1998); Jumlongras, D. et al. A nonsense mutation in MSX1 causes Witkop syndrome Am J Hum Genet. 69, 67-74 (2001)). Ablation or ectopic expression of transcription factors in mouse models such as Engrailed have also provided insights into nail development (Loomis, C. A. et al. The mouse Engrailed-1 gene and ventral limb patterning. Nature 382, 360-3 (1996)).

As used herein, the term “subject” is used to refer to any animal that would normally possess keratin containing limb appendages, such as nails, hooves or claws. Thus, included within the scope of the “subjects” of the invention are individual animals that do not posses keratin containing limb appendages, such as nails, hooves or claws, for example as the result of a disease or inherited abnormality. Any type of animal that possesses, or should possess keratin containing limb appendages may be a subject, including, but not limited to mammals, reptiles, and birds. For example, mammalian subjects of the invention include, but are not limited to, humans, cats, dogs, cows, horses, sheep, goats and the like. Avian subjects of the invention include, for example, chickens. The subjects of the invention may be wild animals, domestic pets, or animals raised for agriculture or sport. The subjects referred to herein may be in need of treatment to facilitate or enhance the growth and/or strengthen these keratin containing limb appendages. The subjects of the invention may also be in need of, or desirous of, cosmetic enhancement of the keratin containing limb appendages. For example, in the case of human subjects, the subjects may desire a cosmetic treatment to enhance the appearance, strength, or growth rate of their nails. The subjects referred to herein may also be in need of, or desirous of, treatment to inhibit or decrease the growth and/or strength of these keratin containing limb appendages. For example, in some embodiments, the subjects of the invention may be animals, such as cats or dogs, in need of claw reduction or removal. The methods and compositions of the invention may be useful for inhibiting the growth of claws, thereby reducing or eliminating the need for de-clawing. Similarly, the methods and compositions of the invention may be useful for reducing the strength of claws, thereby facilitating removal or trimming of claws.

As used herein, the term “keratin-containing limb appendage” includes nails, hooves, claws, talons, and the like.

As used herein, the term “keratin-related abnormality” is used to refer to any disease, disorder, syndrome or condition that affects keratin-containing limb appendages. Such abnormalities may involve, for example, absence, loss, reduced size, reduced strength, reduced growth or malformation of at least one keratin-containing appendage in a subject. The term “keratin-related abnormality” includes inherited genetic abnormalities. For example, there are various human genetic disorders that are associated with nail abnormalities, including congenital anonychia, congenital hyponychia, Cooks syndrome, nail patella syndrome, ectodermal dysplasias, epidermolysis bullosa, Witkop syndrome, and the like. Also, included within the scope of the present invention, are abnormalities associated with, or caused by, aberrant expression of R-spondin 4 protein, including, but not limited to, congenital anonychia. These genetic diseases are within the scope of the invention, as are other similar human and animal inherited diseases. The term “keratin-related abnormality” also includes infections, such as bacterial, fungal, viral, and parasitic infections, conditions caused by nutritional deficiencies, including, but not limited to iron and calcium deficiencies, and damage or disformity caused by traumatic injury, mechanical injury, burns and the like. For example, accidental injury to human nails, including complete loss of one or more nails, or breakage of one or more nails is included within the meaning of abnormality. Other types of abnormalities that are within the scope of the present invention include, but are not limited to, psoriasis, eczema and koilonychias.

As used herein, the terms “treat”, “treating” or “treatment”, refer to processes intended to cure, ameliorate, reduce the symptoms of, reduce the duration of, or facilitate recovery from, an abnormality.

The R-spondins are proteins involved in Wnt and Frizzled signaling, such as those described in WO/2205/040418, and in the following publications: Nam et al. Mouse cristin/R-spondin family proteins are novel ligands for the Frizzled 8 and LRP6 receptors and activate beta-catenin-dependent gene expression. J Biol. Chem. 2006 May 12; 281(19):13247-57; Kim et al. R-Spondin proteins: a novel link to beta-catenin activation. Cell Cycle. 2006 January; 5(1):23-6; and Kamata et al, R-spondin, a novel gene with thrombospondin type 1 domain, was expressed in the dorsal neural tube and affected in Wnt mutants. Biochim Biophys Acta. 2004 Jan. 5; 1676(1):51-62.

Wnts are secreted from cells, however rarely as a soluble form (Papkoff J and B Schryver, Mol Cell Biol, 1990, 10:2723-30; Burrus L W and McMahon A P, Exp Cell Res, 1995, 220:363-73; Willert K, et al., Nature, 2003 423:448-52). Wnt proteins are glycosylated (Mason J O, et al., Mol Biol Cell, 1992, 3:521-33) and palmitoylated (Willert K, et al., Nature, 2003 423:448-52). In the Wnt signaling pathway, Wnt binds to Frizzled (Frz), a cell surface receptor that is found on various cell types. In the presence of Dishevelled (Dsh), binding of Wnt to the Frz receptor purportedly results in inhibiting GSK3β mediated phosphorylation. Inhibition of this phosphorylation event allegedly would then subsequently halt phosphorylation-dependent degradation of β-catenin. Thus, Wnt binding stabilizes cellular β-catenin. β-catenin can then accumulate in the cytoplasm in the presence of Wnt binding and can subsequently bind to a transcription factor, such as Lef1. The β-catenin-Lef1 complex is then capable of translocating to the nucleus, where the β-catenin-Lef1 complex can mediate transcriptional activation. Other effects and components of the Wnt signaling pathway are described in the following: Arias A M, et al., Curr Opin GenetDev, 1999, 9: 447-454; Nusse R, Development, 2003, 130(22):5297-305; Nelson W J and R Nusse, Science, 2004, 303:1483-7; Logan C Y and R Nusse, Annu Rev Cell Dev Biol, 2004, 20:781-810; Moon R T, et al., Nat Rev Genet, 2004, 5(9):691-701; Brennan K R and A M Brown, J Mammary Gland Biol Neoplasia, 2004, 9(2):119-31; Johnson M L, et al., Bone Miner Res, 2004, 19(11):1749-57; Nusse R, Nature, 2005, 438:747-9; Reya T and H Clevers Nature, 2005, 434:843-50; Gregorieff A and H Clevers, Genes Dev, 2005, 19(8):877-90; Bejsovec A, Cell, 2005, 120(1):11-4; Brembeck F H, et al., Curr Opin Genet Dev, 2006, 16(1):51-9 which are herein incorporated by reference.

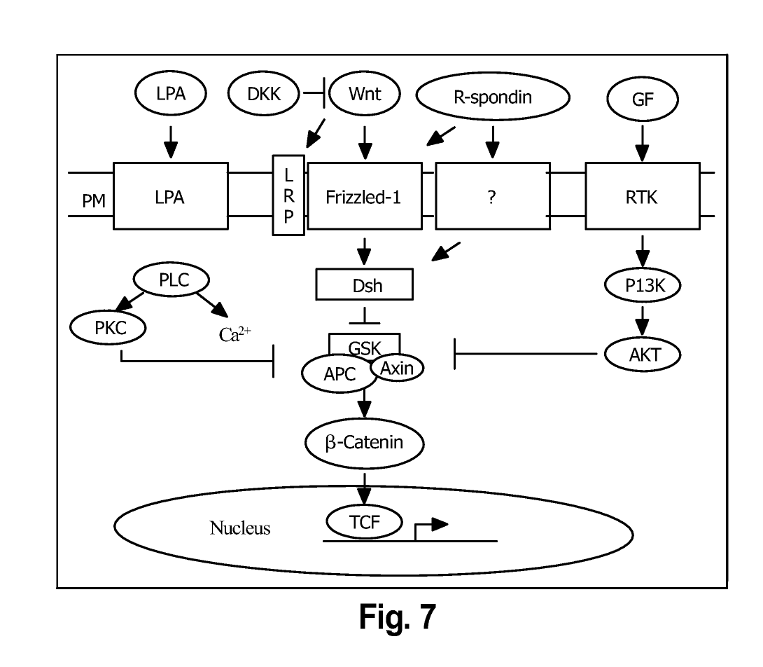

There are currently four human R-spondin family members known, termed R-spondin 1, 2, 3, and 4. These proteins can also be referred to as Futrins, such as Futrin 1, 2, 3, or 4. The R-spondin proteins are encoded by the RSPO genes, such as the RSPO1, 2, 3, and 4 genes. R-spondin 4 (RSPO4), a secreted protein implicated in Wnt signaling, is mutated in inherited anonychia. RSPO4 is a member of the R-spondin family, which comprises four distinct secreted proteins. R-spondins are purported activators of β-catenin signaling (see novel frizzled ligands, discussed in Nam et al. 2006 and FIG. 7). For example, RSPO-1 is reported to be involved in the stimulation of epithelial proliferation in the mammalian intestine. RSPO-4, for example, here is reported to be expressed in the digit tip mesenchyme, and stimulates proliferation of the overlying epithelial cells of the nail plate, potentially via maintaining a Wnt signal.

Provided herein are the amino acid and nucleotide sequences of human R-spondin 4, having SEQ ID NO: 1 and SEQ ID NO: 2, respectively. R-spondin 4 nucleotide and amino acid sequences are also available in GenBank and have deposit numbers NM_{—}001029871 and NP_{—}001025042, respectively.

SEQ ID NO: 1 is the human wild type amino acid sequence corresponding to R-spondin 4 (residues 1-234):

MRAPLCLLLL VAHAVDMLAL NRRKKQVGTG LGGNCTGCII
CSEENGCSTC QQRLFLFIRR EGIRQYGKCL HDCPPGYFGI
RGQEVNRCKK CGATCESCFS QDFCIRCKRQ FYLYKGKCLP
TCPPGTLAHQ NTRECQGECE LGRWGGWSRC THNGKTCGSA
WGLESRVREA GRAGHEEAAT CQVLSESRKC PIQRPCPGER
SPGQKKGRKD RRPRKDRKLD RRLDVRPRQP GLQP

Exon 1 of R-spondin 4 comprises amino acids at positions of about 1 through about 27. Exon 2 of R-spondin 4 (underlined) comprises amino acids at positions of about 28 through about 90. Exon 3 of R-spondin 4 (Bold) comprises amino acids at positions of about 91 through about 137. Exon 4 of R-spondin 4 (Italics) comprises amino acids at positions of about 138 through about 199. Exon 5 of R-spondin 4 (bold and underlined) comprises amino acids at positions of about 200 through about 234.

SEQ ID NO: 2 is the human wild type nucleic acid sequence corresponding to R-spondin 4 (residues −9 to 705):

⁻⁹GCT GCC CAG ⁺¹ATG CGG GCG CCA CTC TGC CTG CTC
CTG CTC GTC GCC CAC GCC GTG GAC ATG CTC GCC CTG
AAC CGA AGG AAG AAG CAA GTG GGC ACT GGC CTG GGG
GGC AAC TGC ACA GGC TGT ATC ATC TGC TCA GAG GAG
AAC GGC TGT TCC ACC TGC CAG CAG AGG CTC TTC CTG
TTC ATC CGC CGG GAA GGC ATC CGC CAG TAC GGC TGT
CCC CCT GGG TAC TTC GGC ATC CGC GGC CAG GAG GTC
AAC AGG TGC AAA AAG CTC TTC CTG TTC ATC CGC CGG
GAA GGC ATC CGC CAG TAC GGC AAG TGC CTG CAC GAC
TGT CCC CCT GGG TAC TTC GGC ATC CGC GGC CAG GAG
GTC AAC AGG TGC AAA AAGTGT GGG GCC ACT TGT GAG
AGC TGC TTC AGC CAG GAC TTC TGC ATC CGG TGC AAG
AGG CAG TTT TAC TTG TAC AAG GGG AAG TGT CTG CCC
ACC TGC CCG CCG GGC ACT TTG GCC CAC CAG AAC ACA
CGG GAG TGC CAG GGG GAG TGT GAA CTG GGT CCC TGG
GGC GGC TGG AGC CCC TGC ACA CAC AAT GGA AAG ACC
TGC GGC TCG GCT TGG GGC CTG GAG AGC CGG GTA CGA
GAG GCT GGC CGG GCT GGG CAT GAG GAG GCA GCC ACC
TGC CAG GTG CTT TCT GAG TCA AGG AAA TGT CCC ATC
CAG AGG CCC TGC CCA GGA GAGAGG AGC CCC GGC CAG
AAG AAG GGC AGG AAG GAC CGG CGC CCA CGC AAG GAC
AGG AAG CTG GAC CGC AGG CTG GAC GTG AGG CCG CGC
CAG CCC GGC CTG CAG CCC TGA

Exon 1 of R-spondin 4 precedes exon 2 of R-spondin 4 as underlined text in SEQ ID NO: 2. Exon 3 of R-spondin 4 in SEQ ID NO: 2 is in bold while exon 4 of R-spondin 4 is in italics. Exon 5 of R-spondin 4 in SEQ ID NO: 2 is in bold and underlined.

SEQ ID NO: 18 is the human wild type nucleotide sequence corresponding to R-spondin 4 (nucleotides 1-2722):

CACAGCAGCCCCCGCGCCCGCCGTGCCGCCGCCGGGACGTGGGGCCCTTG
GGCCGTCGGGCCGCCTGGGGAGCGCCAGCCCGGATCCGGCTGCCCAGATG
CGGGCGCCACTCTGCCTGCTCCTGCTCGTCGCCCACGCCGTGGACATGCT
CGCCCTGAACCGAAGGAAGAAGCAAGTGGGCACTGGCCTGGGGGGCAACT
GCACAGGCTGTATCATCTGCTCAGAGGAGAACGGCTGTTCCACCTGCCAG
CAGAGGCTCTTCCTGTTCATCCGCCGGGAAGGCATCCGCCAGTACGGCAA
GTGCCTGCACGACTGTCCCCCTGGGTACTTCGGCATCCGCGGCCAGGAGG
TCAACAGGTGCAAAAAATGTGGGGCCACTTGTGAGAGCTGCTTCAGCCAG
GACTTCTGCATCCGGTGCAAGAGGCAGTTTTACTTGTACAAGGGGAAGTG
TCTGCCCACCTGCCCGCCGGGCACTTTGGCCCACCAGAACACACGGGAGT
GCCAGGGGGAGTGTGAACTGGGTCCCTGGGGCGGCTGGAGCCCCTGCACA
CACAATGGAAAGACCTGCGGCTCGGCTTGGGGCCTGGAGAGCCGGGTACG
AGAGGCTGGCCGGGCTGGGCATGAGGAGGCAGCCACCTGCCAGGTGCTTT
CTGAGTCAAGGAAATGTCCCATCCAGAGGCCCTGCCCAGGAGAGAGGAGC
CCCGGCCAGAAGAAGGGCAGGAAGGACCGGCGCCCACGCAAGGACAGGAA
GCTGGACCGCAGGCTGGACGTGAGGCCGCGCCAGCCCGGCCTGCAGCCCT
GACCGCCGGCTCTCCCGACTCTCTGGTCCTAGTCCTCGGCCCCTGCACAC
CTCCTCCTGCTCCTTCTCCTCCTCTCCTCTTACTCTTTCTCCTCTGTCTT
CTCCATTTGTCCTCTCTTTCTTTCCACCCTTCTATCATTTTTCTGTCAGT
CTACCTTCCCTTTCTTTTTCTTTTTTATTTCCTTTATTTCTTCCACCTCC
ATTCTCCTCTCCTTTCTCCCTCCCTCCTTCCCTTCCTTCCTCTTCTTTCT
CACTTATCTTTTATCTTTCCTTTTCTTTCTTCCTGTGTTTCTTCCTGTCC
TTCACCGCATCCTTCTCTCTCTCCCTCCTCTTGTCTCCCTCTCACACACA
CTTTAAGAGGGACCATGAGCCTGTGCCCTCCCCTGCAGCTTTCTCTATCT
ACAACTTAAAGAAAGCAAACATCTTTTCCCAGGCCTTTCCCTGACCCCAT
CTTTGCAGAGAAAGGGTTTCCAGAGGGCAAAGCTGGGACACAGCACAGGT
GAATCCTGAAGGCCCTGCTTCTGCTCTGGGGGAGGCTCCAGGACCCTGAG
CTGTGAGCACCTGGTTCTCTGGACAGTCCCCAGAGGCCATTTCCACAGCC
TTCAGCCACCAGCCACCCCGAGGAGCTGGCTGGACAAGGCTCCAGGGCTT
CCAGAGGCCTGGCTTGGACACCTCCCCCAGCTGGCCGTGGAGGGTCACAA
CCTGGCCTCTGGGTGGGCAGCCAGCCCTGGAGGGCATCCTCTGCAAGCTG
CCTGCCACCCTCATCGGCACTCCCCCACAGGCCTCCCTCTCATGGGTTCC
ATGCCCCTTTTTCCCAAGCCGGATCAGGTGAGCTGTCACTGCTGGGGGAT
CCACCTGCCCAGCCCAGAAGAGGCCACTGAAACGGAAAGGAAAGCTGAGA
TTATCCAGCAGCTCTGTTCCCCACCTCAGCGCTTCCTGCCCATGTGGGGA
AACAGGTCTGAGAAGGAAGGGGCTTGCCCAGGGTCACACAGGAAGCCTTC
AGGCTCTGCTTCTGCCTGATGGCTCTGCTCAGCACATTCACGGTGGAGAG
GAGAATTTGGGGGTCACTTGAGGGGGGAAATGTAGGGAATTGTGGGTGGG
GAGCAAGGGAAGATCCGTGCACTCGTCCACACCCACCACCACACTCGCTG
ACACCCACCCCCACACGCTGACACCCACCCCCACACTTGCCCACACCCAT
CACCGCACTCGCCCACACCCACCACCACACTGCCCCACACCCACCACCAC
ACTCCCCCACACCCACCACCACACTCGCCCACACCCACCACCAGTGACTT
GAGCATCTGTGCTTCGCTGTGACGCCCCTCGCCCTAGGCAGGAACGACGC
TGGGAGGAGTCTCCAGGTCAGACCCAGCTTGGAAGCAAGTCTGTCCTCAC
TGCCTATCCTTCTGCCATCATAACACCCCCTTCCTGCTCTGCTCCCCGGA
ATCCTCAGAAACGGGATTTGTATTTGCCGTGACTGGTTGGCCTGAACACG
TAGGGCTCCGTGACTGGGACAGGAATGGGCAGGAGAAGCAAGAGTCGGAG
CTCCAAGGGGCCCAGGGGTGGCCTGGGGAAGGAAGATGGTCAGCAGGCTG
GGGGAGAGGCTCTAGGTGATGAAATATTACATTCCCGACCCCAAGAGAGC
ACCCACCCTCAGACCTGCCCTCCACCTGGCAGCTGGGGAGCCCTGGCCTG
AACCCCCCCCTCCCAGCAGGCCCACCCTCTCTCTGACTTCCCTGCTCTCA
CCTCCCCGAGAACAGCTAGAGCCCCCTCCTCCGCCTGGCCAGGCCACCAG
CTTCTCTTCTGCAAACGTTTGTGCCTCTGAAATGCTCCGTTGTTATTGTT
TCAAGACCCTAACTTTTTTTTAAAACTTTCTTAATAAAGGGAAAAGAAAC
TTGTAAAAAAAAAAAAAAAAAA

SEQ ID NO: 19 is the human wild type nucleotide sequence (including intron sequences) that corresponds to R-spondin 4 (nucleotides 1-8556):

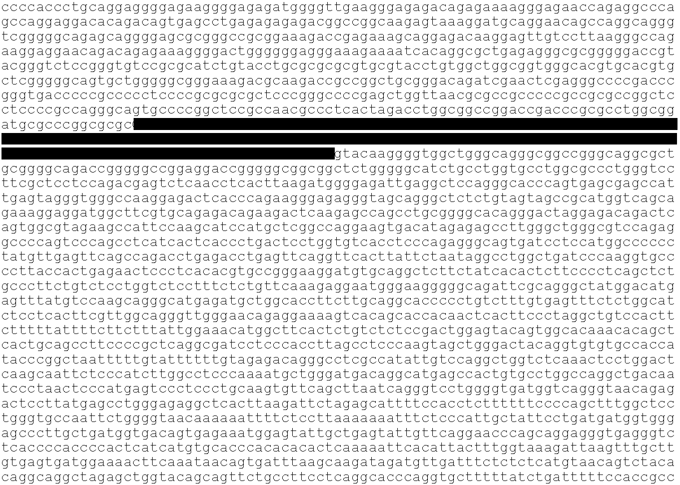

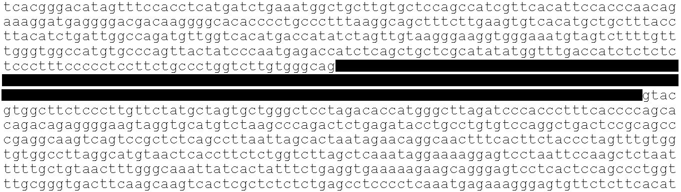

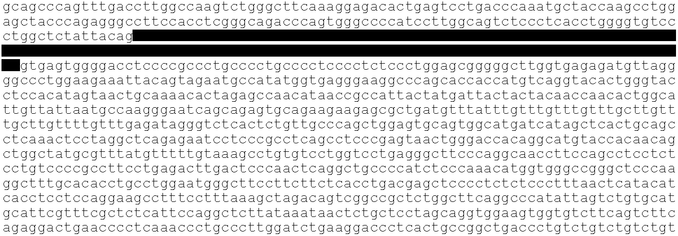

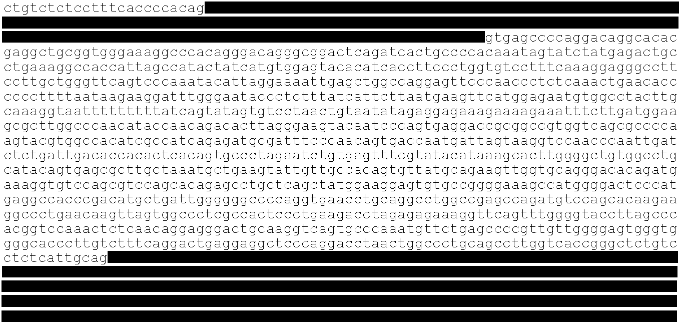

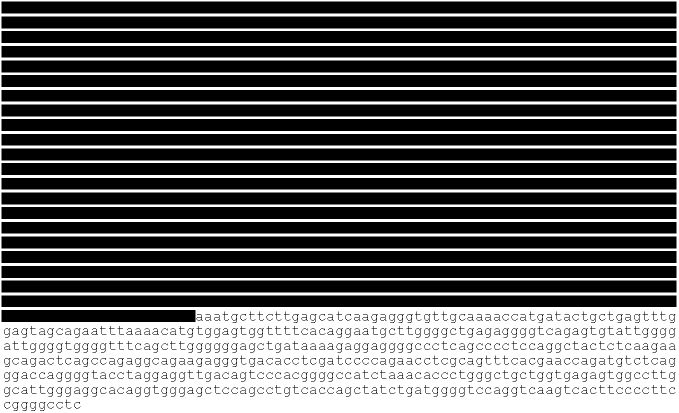

The exon sequences (Exons 1-5, respectively) are shadowed in SEQ ID NO. 19. Exon 1 of R-spondin 4 comprises nucleic acids at positions from about 632 through about 808. Exon 2 of R-spondin 4 comprises nucleic acids at positions from about 2888 through about 3076. Exon 3 of R-spondin 4 comprises nucleic acids at positions from about 3712 through about 3852. Exon 4 of R-spondin 4 comprises nucleic acids at positions from about 4798 through about 4983. Exon 5 of R-spondin 4 comprises nucleic acids at positions from about 6096 through about 6351.

According to the invention, R-spondin 4 molecules can comprise polynucleotide molecules or variants thereof, and polypeptide molecules, or variants, fragments, or peptidomimetics thereof. Contemplated variants of the R-spondin 4 proteins described herein include those having at least from about 46% to about 50% identity to SEQ ID NO: 1, or having at least from about 50.1% to about 55% identity to SEQ ID NO: 1, or having at least from about 55.1% to about 60% identity to SEQ ID NO: 1, or having from at least about 60.1% to about 65% identity to SEQ ID NO: 1, or having from about 65.1% to about 70% identity to SEQ ID NO: 1, or having at least from about 70.1% to about 75% identity to SEQ ID NO: 1, or having at least from about 75.1% to about 80% identity to SEQ ID NO: 1, or having at least from about 80.1% to about 85% identity to SEQ ID NO: 1, or having at least from about 85.1% to about 90% identity to SEQ ID NO: 1, or having at least from about 90.1% to about 95% identity to SEQ ID NO: 1, or having at least from about 95.1% to about 97% identity to SEQ ID NO: 1, or having at least from about 97.1% to about 99% identity to SEQ ID NO: 1.

DNA and Amino Acid Manipulation Methods and Purification Thereof

The present invention is also directed isolated nucleic acids encoding any one of the R-spondin 4 polypeptide molecules, variants, or fragments thereof. It utilizes conventional molecular biology, microbiology, and recombinant DNA techniques available to one of ordinary skill in the art. Such techniques are well known to the skilled worker and are explained fully in the literature. See, e.g., Maniatis, Fritsch & Sambrook, “Molecular Cloning: A Laboratory Manual” (1982): “DNA Cloning: A Practical Approach,” Volumes I and II (D. N. Glover, ed., 1985); “Oligonucleotide Synthesis” (M. J. Gait, ed., 1984); “Nucleic Acid Hybridization” (B. D. Hames & S. J. Higgins, eds., 1985); “Transcription and Translation” (B. D. Hames & S. J. Higgins, eds., 1984); “Animal Cell Culture” (R. I. Freshney, ed., 1986); “Immobilized Cells and Enzymes” (IRL Press, 1986): B. Perbal, “A Practical Guide to Molecular Cloning” (1984), and Sambrook, et al., “Molecular Cloning: a Laboratory Manual” (1989).

Programs and algorithms for sequence alignment and comparison of % identity and/or homology between nucleic acid sequences, or polypeptides, are well known in the art, and include BLAST, SIM alignment tool, and so forth. TBLAST Program (Altschul, S. F., et al., “Basic Local Alignment Search Tool,” J. Mol. Biol. 215:403 410 (1990).

The invention provides for a nucleic acid encoding a R-spondin 4 polypeptide molecule having at least 46%, 48%, 50%, 55%, 60%, 70%, 75%, 80%, 90%, 95%, or 99% identity to SEQ ID NO: 1. The invention also provides for a nucleic acid encoding a R-spondin 4 polypeptide molecule fragment or variant thereof. In one embodiment, the nucleic acid molecule is expressed in an expression cassette, for example to achieve overexpression in a cell. The nucleic acids of the invention can be an RNA, cDNA, cDNA-like, or a DNA nucleic acid molecule of interest in an expressible format, such as an expression cassette, which can be expressed from the natural promoter or a derivative thereof or an entirely heterologous promoter. Alternatively, the nucleic acid molecule of interest can encode an antisense RNA or a silencing RNA (siRNA). For example, the antisense RNA or siRNA molecule can be directed to a particular portion of R-spondin 4 (such as exon 1, exon 2, exon 3, exon 4, or exon 5, which can comprise SEQ ID NO: 27, 28, 29, 30, or 31, respectively). The nucleic acid of interest can encode a R-spondin 4 polypeptide molecule having 46%, 48%, 50%, 55%, 60%, 70%, 75%, 80%, 90%, 95%, or 99% identity to SEQ ID NO: 2, and may or may not include introns.

Protein variants are well understood to those of skill in the art and can involve amino acid sequence modifications. For example, amino acid sequence modifications typically fall into one or more of three classes: substitutional, insertional or deletional variants. Insertions can include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site-specific mutagenesis of nucleotides in the DNA encoding the protein, thereby producing DNA encoding the variant, and thereafter expressing the DNA in recombinant cell culture.

Techniques for making substitution mutations at predetermined sites in DNA having a known sequence are well known, for example M13 primer mutagenesis and PCR mutagenesis. Amino acid substitutions are typically of single residues, but can occur at a number of different locations at once. Insertions usually can be on the order of about from 1 to 10 amino acid residues, while deletions can range about from 1 to 30 residues. Deletions or insertions can be made in adjacent pairs (for example, a deletion of 2 residues or insertion of 2 residues). Substitutions, deletions, insertions or any combination thereof can be combined to arrive at a final construct. The mutations must not place the sequence out of reading frame and should not create complementary regions that could produce secondary mRNA structure. Substitutional variants are those in which at least one residue has been removed and a different residue inserted in its place.

In one embodiment, an isolated mutant human R-spondin 4 polypeptide can comprise a Q>R mutation at amino acid position 65 of SEQ ID NO: 1. The R-spondin 4 Q>R mutant can comprise the amino acid sequence of SEQ ID NO: 3, wherein the mutation is found in exon 2 of R-spondin 4.

SEQ ID NO: 3 is the human mutant amino acid sequence corresponding to the R-spondin 4 Q>R mutation at amino acid position 65 (bold and underlined) of SEQ ID NO: 1 (residues 1-234):

MRAPLCLLLLVAHAVDMLALNRRKKQVGTGLGGNCTGCIICSEENGCSTC
QQRLFLFIRREGIRRYGKCLHDCPPGYFGIRGQEVNRCKKCGATCESCFS
QDFCIRCKRQFYLYKGKCLPTCPPGTLAHQNTRECQGECELGPWGGWSPC
THNGKTCGSAWGLESRVREAGRAGHEEAATCQVLSESRKCPIQRPCPGER
SPGQKKGRKDRRPRKDRKLDRRLDVRPRQPGLQP

SEQ ID NO: 7 (residues 1-705) is the human mutant nucleic acid sequence corresponding to the R-spondin 4 A>G mutation at nucleic acid position 194 (bold and underlined) of SEQ ID NO: 2:

ATGCGGGCGCCACTCTGCCTGCTCCTGCTCGTCGCCCACGCCGTGGACAT
GCTCGCCCTGAACCGAAGGAAGAAGCAAGTGGGCACTGGCCTGGGGGGCA
ACTGCACAGGCTGTATCATCTGCTCAGAGGAGAACGGCTGTTCCACCTGC
CAGCAGAGGCTCTTCCTGTTCATCCGCCGGGAAGGCATCCGCCGGTACGG
CAAGTGCCTGCACGACTGTCCCCCTGGGTACTTCGGCATCCGCGGCCAGG
AGGTCAACAGGTGCAAAAAGTGTGGGGCCACTTGTGAGAGCTGCTTCAGC
CAGGACTTCTGCATCCGGTGCAAGAGGCAGTTTTACTTGTACAAGGGGAA
GTGTCTGCCCACCTGCCCGCCGGGCACTTTGGCCCACCAGAACACACGGG
AGTGCCAGGGGGAGTGTGAACTGGGTCCCTGGGGCGGCTGGAGCCCCTGC
ACACACAATGGAAAGACCTGCGGCTCGGCTTGGGGCCTGGAGAGCCGGGT
ACGAGAGGCTGGCCGGGCTGGGCATGAGGAGGCAGCCACCTGCCAGGTGC
TTTCTGAGTCAAGGAAATGTCCCATCCAGAGGCCCTGCCCAGGAGAGAGG
AGCCCCGGCCAGAAGAAGGGCAGGAAGGACCGGCGCCCACGCAAGGACAG
GAAGCTGGACCGCAGGCTGGACGTGAGGCCGCGCCAGCCCGGCCTGCAGC
CCTGA

In another embodiment, an isolated mutant human R-spondin 4 polypeptide can comprise a C>F mutation at amino acid position 95 (bold and underlined) of SEQ ID NO: 1. The R-spondin 4 C>F mutant can comprise the amino acid sequence of SEQ ID NO: 4, wherein the mutation is found in exon 3 of R-spondin 4.

SEQ ID NO: 4 is the human mutant amino acid sequence corresponding to the R-spondin 4 C>F mutation at amino acid position 95 of SEQ ID NO: 1 (residues 1-234):

MRAPLCLLLLVAHAVDMLALNRRKKQVGTGLGGNCTGCIICSEENGCSTC
QQRLFLFIRREGIRQYGKCLHDCPPGYFGIRGQEVNRCKKCGATFESCFS
QDFCIRCKRQFYLYKGKCLPTCPPGTLAHQNTRECQGECELGPWGGWSPC
THNGKTCGSAWGLESRVREAGRAGHEEAATCQVLSESRKCPIQRPCPGER
SPGQKKGRKDRRPRKDRKLDRRLDVRPRQPGLQP

SEQ ID NO: 8 (residues 1-705) is the human mutant nucleic acid sequence corresponding to the R-spondin 4 G>T mutation at nucleic acid position 284 (bold and underlined) of SEQ ID NO: 2:

ATGCGGGCGCCACTCTGCCTGCTCCTGCTCGTCGCCCACGCCGTGGACAT
GCTCGCCCTGAACCGAAGGAAGAAGCAAGTGGGCACTGGCCTGGGGGGCA
ACTGCACAGGCTGTATCATCTGCTCAGAGGAGAACGGCTGTTCCACCTGC
CAGCAGAGGCTCTTCCTGTTCATCCGCCGGGAAGGCATCCGCCAGTACGG
CAAGTGCCTGCACGACTGTCCCCCTGGGTACTTCGGCATCCGCGGCCAGG
AGGTCAACAGGTGCAAAAAGTGTGGGGCCACTTTTGAGAGCTGCTTCAGC
CAGGACTTCTGCATCCGGTGCAAGAGGCAGTTTTACTTGTACAAGGGGAA
GTGTCTGCCCACCTGCCCGCCGGGCACTTTGGCCCACCAGAACACACGGG
AGTGCCAGGGGGAGTGTGAACTGGGTCCCTGGGGCGGCTGGAGCCCCTGC
ACACACAATGGAAAGACCTGCGGCTCGGCTTGGGGCCTGGAGAGCCGGGT
ACGAGAGGCTGGCCGGGCTGGGCATGAGGAGGCAGCCACCTGCCAGGTGC
TTTCTGAGTCAAGGAAATGTCCCATCCAGAGGCCCTGCCCAGGAGAGAGG
AGCCCCGGCCAGAAGAAGGGCAGGAAGGACCGGCGCCCACGCAAGGACAG
GAAGCTGGACCGCAGGCTGGACGTGAGGCCGCGCCAGCCCGGCCTGCAGC
CCTGA

In a further embodiment, an isolated mutant human R-spondin 4 polypeptide can comprise a C>R mutation at amino acid position 107 of SEQ ID NO: 1. The R-spondin 4 C>R mutant can comprise the amino acid sequence of SEQ ID NO: 5, wherein the mutation is found in exon 3 of R-spondin 4.

SEQ ID NO: 5 is the human mutant amino acid sequence corresponding to the R-spondin 4 C>R mutation at amino acid position 107 (bold and underlined) of SEQ ID NO: 1 (residues 1-234):

MRAPLCLLLLVAHAVDMLALNRRKKQVGTGLGGNCTGCIICSEENGCSTC
QQRLFLFIRREGIRQYGKCLHDCPPGYFGIRGQEVNRCKKCGATCESCFS
QDFCIRRKRQFYLYKGKCLPTCPPGTLAHQNTRECQGECELGPWGGWSPC
THNGKTCGSAWGLESRVREAGRAGHEEAATCQVLSESRKCPIQRPCPGER
SPGQKKGRKDRRPRKDRKLDRRLDVRPRQPGLQP

SEQ ID NO: 9 (residues 1-705) is the human mutant nucleic acid sequence corresponding to the R-spondin 4 T>C mutation at nucleic acid position 319 (bold and underlined) of SEQ ID NO: 2:

ATGCGGGCGCCACTCTGCCTGCTCCTGCTCGTCGCCCACGCCGTGGACAT
GCTCGCCCTGAACCGAAGGAAGAAGCAAGTGGGCACTGGCCTGGGGGGCA
ACTGCACAGGCTGTATCATCTGCTCAGAGGAGAACGGCTGTTCCACCTGC
CAGCAGAGGCTCTTCCTGTTCATCCGCCGGGAAGGCATCCGCCAGTACGG
CAAGTGCCTGCACGACTGTCCCCCTGGGTACTTCGGCATCCGCGGCCAGG
AGGTCAACAGGTGCAAAAAGTGTGGGGCCACTTGTGAGAGCTGCTTCAGC
CAGGACTTCTGCATCCGGCGCAAGAGGCAGTTTTACTTGTACAAGGGGAA
GTGTCTGCCCACCTGCCCGCCGGGCACTTTGGCCCACCAGAACACACGGG
AGTGCCAGGGGGAGTGTGAACTGGGTCCCTGGGGCGGCTGGAGCCCCTGC
ACACACAATGGAAAGACCTGCGGCTCGGCTTGGGGCCTGGAGAGCCGGGT
ACGAGAGGCTGGCCGGGCTGGGCATGAGGAGGCAGCCACCTGCCAGGTGC
TTTCTGAGTCAAGGAAATGTCCCATCCAGAGGCCCTGCCCAGGAGAGAGG
AGCCCCGGCCAGAAGAAGGGCAGGAAGGACCGGCGCCCACGCAAGGACAG
GAAGCTGGACCGCAGGCTGGACGTGAGGCCGCGCCAGCCCGGCCTGCAGC
CCTGA

In some embodiments, an isolated mutant human R-spondin 4 polypeptide can comprise a C>Y mutation at amino acid position 118 of SEQ ID NO: 1. The R-spondin 4 C>Y mutant can comprise the amino acid sequence of SEQ ID NO: 6, wherein the mutation is found in exon 3 of R-spondin 4.

SEQ ID NO: 6 is the human mutant amino acid sequence corresponding to the R-spondin 4 C>Y mutation at amino acid position 118 (bold and underlined) of SEQ ID NO: 1 (residues 1-234):

MRAPLCLLLLVAHAVDMLALNRRKKQVGTGLGGNCTGCIICSEENGCSTC
QQRLFLFIRREGIRQYGKCLHDCPPGYFGIRGQEVNRCKKCGATCESCFS
QDFCIRCKRQFYLYKGKYLPTCPPGTLAHQNTRECQGECELGPWGGWSPC
THNGKTCGSAWGLESRVREAGRAGHEEAATCQVLSESRKCPIQRPCPGER
SPGQKKGRKDRRPRKDRKLDRRLDVRPRQPGLQP

SEQ ID NO: 10 (residues 1-705) is the human mutant nucleic acid sequence corresponding to the R-spondin 4 G>A mutation at nucleic acid position 353 (bold and underlined) of SEQ ID NO: 2:

ATGCGGGCGCCACTCTGCCTGCTCCTGCTCGTCGCCCACGCCGTGGACAT
GCTCGCCCTGAACCGAAGGAAGAAGCAAGTGGGCACTGGCCTGGGGGGCA
ACTGCACAGGCTGTATCATCTGCTCAGAGGAGAACGGCTGTTCCACCTGC
CAGCAGAGGCTCTTCCTGTTCATCCGCCGGGAAGGCATCCGCCAGTACGG
CAAGTGCCTGCACGACTGTCCCCCTGGGTACTTCGGCATCCGCGGCCAGG
AGGTCAACAGGTGCAAAAAGTGTGGGGCCACTTGTGAGAGCTGCTTCAGC
CAGGACTTCTGCATCCGGTGCAAGAGGCAGTTTTACTTGTACAAGGGGAA
GTATCTGCCCACCTGCCCGCCGGGCACTTTGGCCCACCAGAACACACGGG
AGTGCCAGGGGGAGTGTGAACTGGGTCCCTGGGGCGGCTGGAGCCCCTGC
ACACACAATGGAAAGACCTGCGGCTCGGCTTGGGGCCTGGAGAGCCGGGT
ACGAGAGGCTGGCCGGGCTGGGCATGAGGAGGCAGCCACCTGCCAGGTGC
TTTCTGAGTCAAGGAAATGTCCCATCCAGAGGCCCTGCCCAGGAGAGAGG
AGCCCCGGCCAGAAGAAGGGCAGGAAGGACCGGCGCCCACGCAAGGACAG
GAAGCTGGACCGCAGGCTGGACGTGAGGCCGCGCCAGCCCGGCCTGCAGC
CCTGA

The invention also provides for isolated human R-spondin 4 mutant molecules that contain an insertional or deletional mutations at its nucleic acid levels. In one embodiment, an isolated mutant human R-spondin 4 polypeptide can be encoded by a nucleic acid sequence comprising at least 46%, 50%, 60%, 70%, 75%, 80%, 90%, 95%, or 99% of SEQ ID NO: 2. In another embodiment, the isolated human R-spondin 4 mutant molecule can comprise the nucleic acid sequence of SEQ ID NO: 11. For example, the nucleic acid sequence of this mutant contains an deletion mutation of 26 nucleotides, GCT GCC CAG ATG CGG GCG CCA CTC TG (SEQ ID NO: 116), starting at position −9 of SEQ ID NO:2, and comprises SEQ ID NO: 11. The deletion of the first ATG codon can result in the deletion of the first 16 amino acids in SEQ ID NO: 1.

SEQ ID NO: 11 is the human mutant nucleic acid sequence corresponding to the R-spondin 4 26-base pair deletion mutant described above (residues 1-688):

C CTG CTC CTG CTC GTC GCC CAC GCC GTG GAC ATG CTC
GCC CTG AAC CGA AGG AAG AAG CAA GTG GGC ACT GGC
CTG GGG GGC AAC TGC ACA GGC TGT ATC ATC TGC TCA
GAG GAG AAC GGC TGT TCC ACC TGC CAG CAG AGG CTC
TTC CTG TTC ATC CGC CGG GAA GGC ATC CGC CAG TAC
GGC AAG TGC CTG CAC GAC TGT CCC CCT GGG TAC TTC
GGC ATC CGC GGC CAG GAG GTC AAC AGG TGC AAA AAG
TGT GGG GCC ACT TGT GAG AGC TGC TTC AGC CAG GAC
TTC TGC ATC CGG TGC AAG AGG CAG TTT TAC TTG TAC
AAG GGG AAG TGT CTG CCC ACC TGC CCG CCG GGC ACT
TTG GCC CAC CAG AAC ACA CGG GAG TGC CAG GGG GAG
TGT GAA CTG GGT CCC TGG GGC GGC TGG AGC CCC TGC
ACA CAC AAT GGA AAG ACC TGC GGC TCG GCT TGG GGC
CTG GAG AGC CGG GTA CGA GAG GCT GGC CGG GCT GGG
CAT GAG GAG GCA GCC ACC TGC CAG GTG CTT TCT GAG
TCA AGG AAA TGT CCC ATC CAG AGG CCC TGC CCA GGA
GAG AGG AGC CCC GGC CAG AAG AAG GGC AGG AAG GAC
CGG CGC CCA CGC AAG GAC AGG AAG CTG GAC CGC AGG
CTG GAC GTG AGG CCG CGC CAG CCC GGC CTG CAG CCC
TGA

In a further embodiment, the isolated human R-spondin 4 mutant molecule can comprise the nucleic acid sequence of SEQ ID NO: 12. For example, the nucleic acid sequence of this mutant contains a deletion mutation of 16 nucleotides, GCT GCC CAG ATG CGG GCG CCA CTC TG (SEQ ID NO: 116), starting at position 95 of SEQ ID NO:2, and comprises SEQ ID NO: 12.

SEQ ID NO: 12 is the human mutant nucleic acid sequence corresponding to the R-spondin 4 sixteen-base pair deletion mutant described above (residues 1-657):

ATGCGGGCGCCACTCTGCCTGCTCCTGCTCGTCGCCCACGCCGTGGACAT
GCTCGCCCTGAACCGAAGGAAGAAGCAAGTGGGCACTGGCCTGGCTGTAT
CATCTGCTCAGAGGAGAACGGCTGTTCCACCTGCCAGCAGAGGCTCTTCC
TGTTCATCCGCCGGGAAGGCATCCGCCAGTACGGCAAGTGCCTGCACGAC
TGTCCCCCTGGGTACTTCGGCATCCGCGGCCAGGAGGTCAACAGGTGCAA
AAAGTGTGGGGCCACTTGTGAGAGCTGCTTCAGCCAGGACTTCTGCATCC
GGTGCAAGAGGCAGTTTTACTTGTACAAGGGGAAGTGTCTGCCCACCTGC
CCGCCGGGCACTTTGGCCCACCAGAACACACGGGAGTGCCAGGGGGAGTG
TGAACTGGGTCCCTGGGGCGGCTGGAGCCCCTGCACACACAATGGAAAGA
CCTGCGGCTCGGCTTGGGGCCTGGAGAGCCGGGTACGAGAGGCTGGCCGG
GCTGGGCATGAGGAGGCAGCCACCTGCCAGGTGCTTTCTGAGTCAAGGAA
ATGTCCCATCCAGAGGCCCTGCCCAGGAGAGAGGAGCCCCGGCCAGAAGA
AGGGCAGGAAGGACCGGCGCCCACGCAAGGACAGGAAGCTGGACCGCAGG
CTGGACG

The deletion of the 16 base pairs from nucleotide at position 95 to nucleotide at position 110 can result in a truncated RSPO4 protein having SEQ ID NO: 13 (residues 1-219):

MRAPLCLLLLVAHAVDMLALNRRKKQVGTGLAVSSAQRRTAVPPASRGSS
CSSAGKASASTASACTTVPLGTSASAARRSTGAKSVGPLVRAASARTSAS
GARGSFTCTRGSVCPPARRALWPTRTHGSARGSVNWVPGAAGAPAHTMER
PAARLGAWRAGYERLAGLGMRRQPPARCFLSQGNVPSRGPAQERGAPARR
RAGRTGAHARTGSWTAGWT

In other embodiments, an isolated mutant human R-spondin 4 polypeptide can comprise a M>T mutation at amino acid position 1 of SEQ ID NO: 1. The R-spondin 4 M>T mutant can comprise the amino acid sequence of SEQ ID NO: 14, wherein the mutation is found in exon 1 of R-spondin 4. The missense mutation, wherein an amino acid substitution occurs at the first methionine start site for isoleucine, can result in the deletion of the first 16 amino acids in SEQ ID NO: 1.

SEQ ID NO: 14 is the human mutant amino acid sequence corresponding to the R-spondin 4 M>I mutation at amino acid position 1 (bold and underlined) of SEQ ID NO: 1 (residues 1-234):

IRAPLCLLLLVAHAVDMLALNRRKKQVGTGLGGNCTGCIICSEENGCSTC
QQRLFLFIRREGIRQYGKCLHDCPPGYFGIRGQEVNRCKKCGATCESCFS
QDFCIRCKRQFYLYKGKCLPTCPPGTLAHQNTRECQGECELGPWGGWSPC
THNGKTCGSAWGLESRVREAGRAGHEEAATCQVLSESRKCPIQRPCPGER
SPGQKKGRKDRRPRKDRKLDRRLDVRPRQPGLQP

SEQ ID NO: 15 (residues 1-705) is the human mutant nucleic acid sequence corresponding to the R-spondin 4 G>A mutation at nucleic acid position 3 (bold and underlined) of SEQ ID NO: 2:

ATACGGGCGCCACTCTGCCTGCTCCTGCTCGTCGCCCACGCCGTGGACAT
GCTCGCCCTGAACCGAAGGAAGAAGCAAGTGGGCACTGGCCTGGGGGGCA
ACTGCACAGGCTGTATCATCTGCTCAGAGGAGAACGGCTGTTCCACCTGC
CAGCAGAGGCTCTTCCTGTTCATCCGCCGGGAAGGCATCCGCCAGTACGG
CAAGTGCCTGCACGACTGTCCCCCTGGGTACTTCGGCATCCGCGGCCAGG
AGGTCAACAGGTGCAAAAAGTGTGGGGCCACTTGTGAGAGCTGCTTCAGC
CAGGACTTCTGCATCCGGTGCAAGAGGCAGTTTTACTTGTACAAGGGGAA
GTGTCTGCCCACCTGCCCGCCGGGCACTTTGGCCCACCAGAACACACGGG
AGTGCCAGGGGGAGTGTGAACTGGGTCCCTGGGGCGGCTGGAGCCCCTGC
ACACACAATGGAAAGACCTGCGGCTCGGCTTGGGGCCTGGAGAGCCGGGT
ACGAGAGGCTGGCCGGGCTGGGCATGAGGAGGCAGCCACCTGCCAGGTGC
TTTCTGAGTCAAGGAAATGTCCCATCCAGAGGCCCTGCCCAGGAGAGAGG
AGCCCCGGCCAGAAGAAGGGCAGGAAGGACCGGCGCCCACGCAAGGACAG
GAAGCTGGACCGCAGGCTGGACGTGAGGCCGCGCCAGCCCGGCCTGCAGC
CCTGA

In some embodiments, the isolated human R-spondin 4 mutant molecule can comprise the nucleic acid sequence of SEQ ID NO: 16. For example, the nucleic acid sequence of this mutant contains a G>A mutation at the exon 2-intron 2 boundary (position 3077 of SEQ ID NO: 19; see reference IVS2+1G>A in Table 3), which comprises SEQ ID NO: 16, and generates a splice site mutant predicted to result in aberrant splicing of RSPO4.

SEQ ID NO: 16 is the human mutant nucleic acid sequence corresponding to the R-spondin 4 G>A mutation at nucleic acid position 3077 (bold and underlined) of SEQ ID NO: 19 (nucleotides 1-8556):

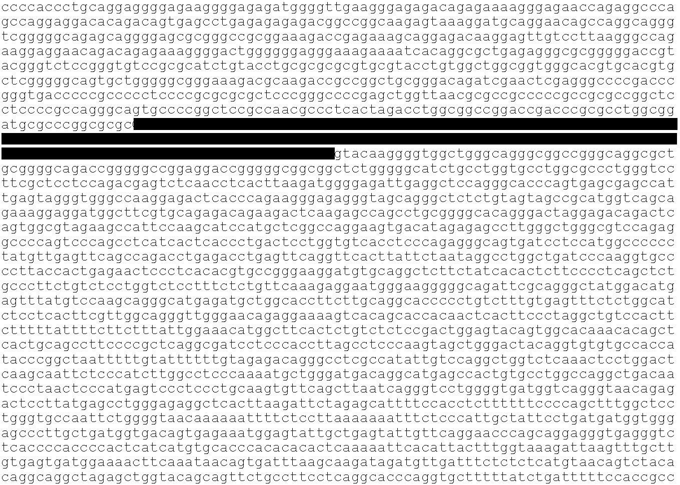

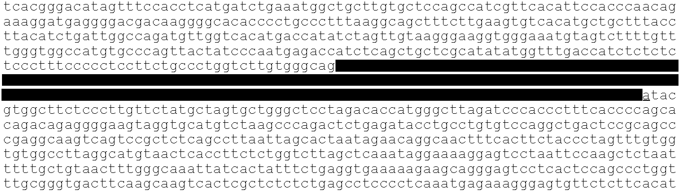

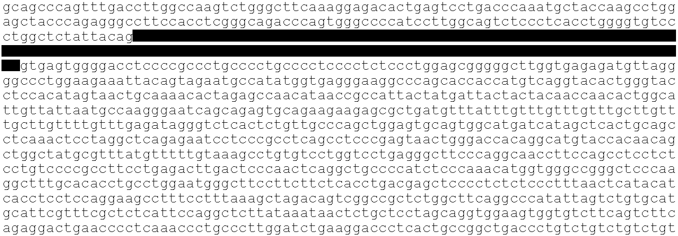

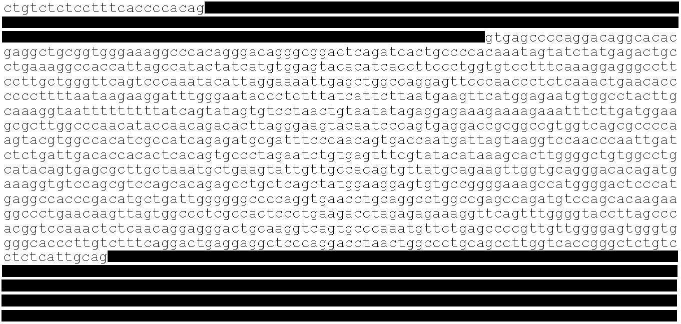

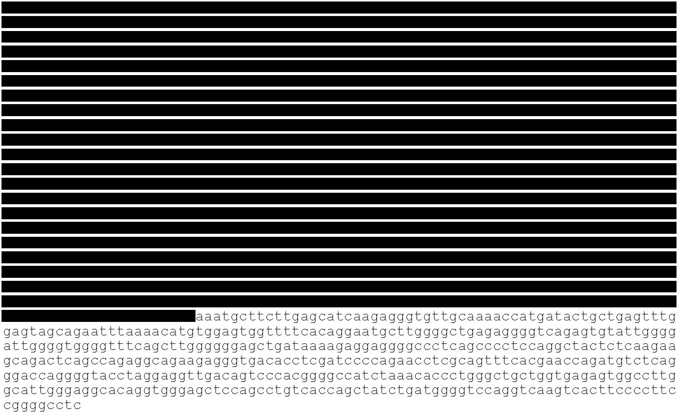

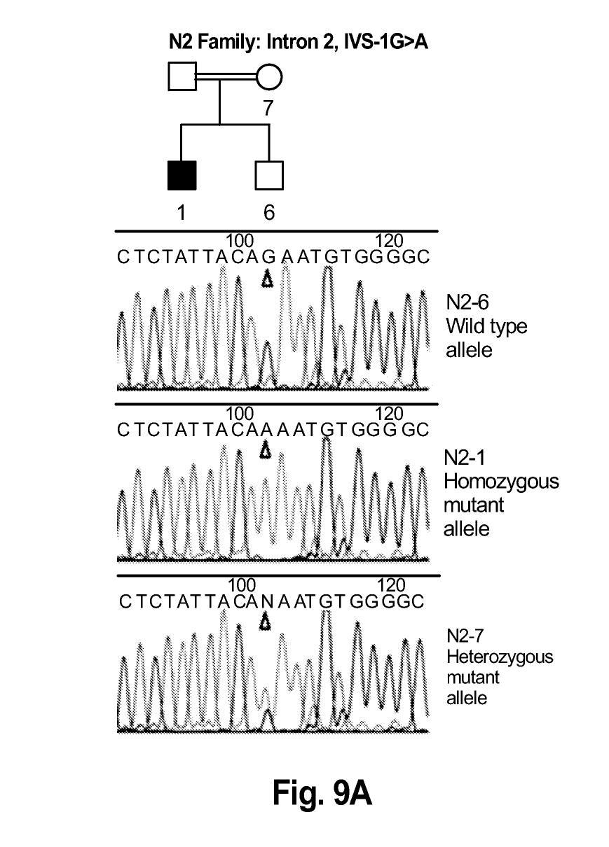

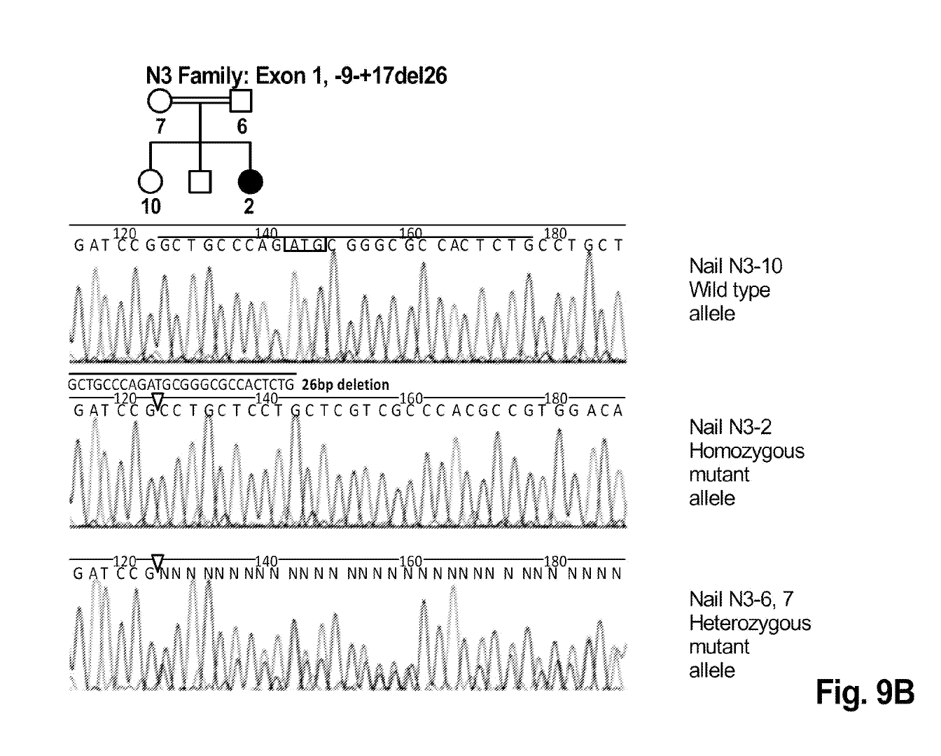

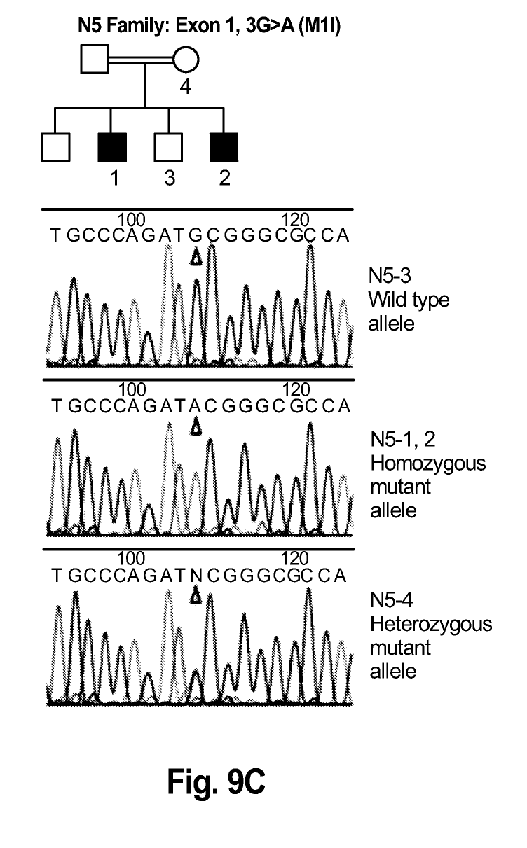

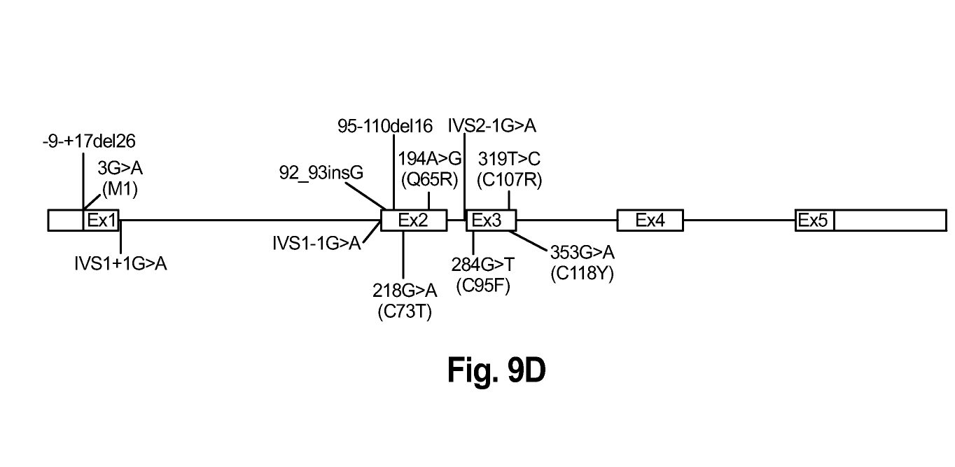

In further embodiments, the isolated human R-spondin 4 mutant molecule can comprise the nucleic acid sequence of SEQ ID NO: 17. For example, the nucleic acid sequence of this mutant contains a G>A mutation at the intron 2-exon 3 boundary (position 3711 of SEQ ID NO: 19; see reference IVS2-1G>A in FIG. 9D), which comprises SEQ ID NO: 17, and generates a splice site mutant predicted to result in aberrant splicing of RSPO4.

SEQ ID NO: 17 is the human mutant nucleic acid sequence corresponding to the R-spondin 4 G>A mutation at nucleic acid position 3711 (bold and underlined) of SEQ ID NO: 19 (nucleotides 1-8556):

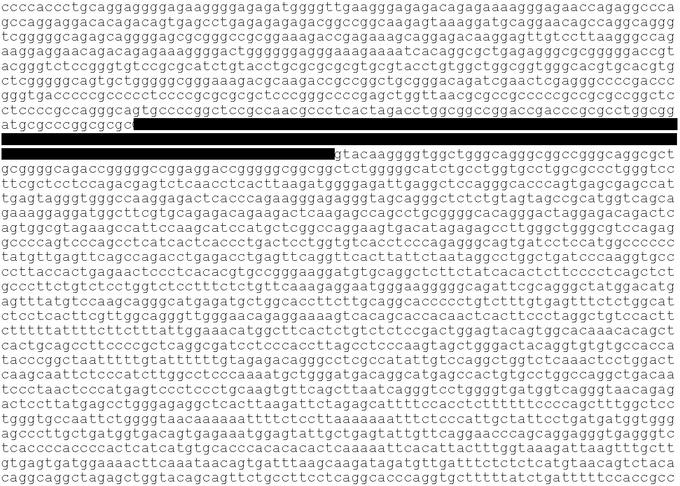

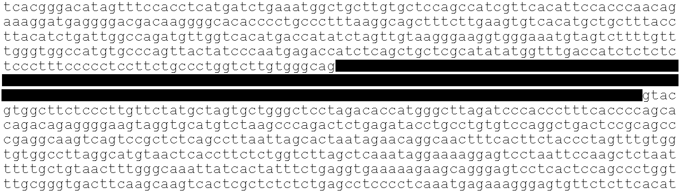

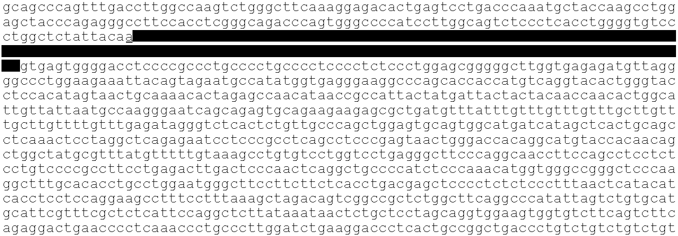

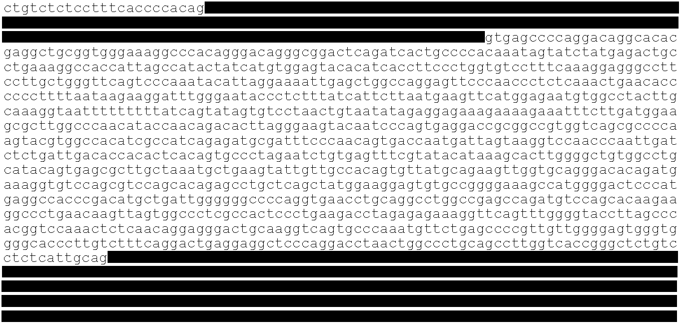

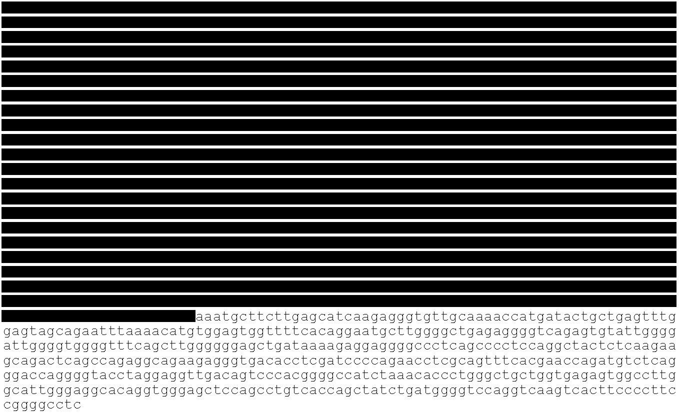

In yet other embodiments, the isolated human R-spondin 4 mutant molecule can comprise the nucleic acid sequence of SEQ ID NO: 20. For example, the nucleic acid sequence of this mutant contains a G>A mutation at the exon 1-intron 1 boundary (position 809 of SEQ ID NO: 19; see reference IVS1+1G>A in FIG. 9D), which comprises SEQ ID NO: 20, and generates a splice site mutant predicted to result in aberrant splicing of RSPO4.

SEQ ID NO: 20 is the human mutant nucleic acid sequence corresponding to the R-spondin 4 G>A mutation at nucleic acid position 809 (bold and underlined) of SEQ ID NO: 19 (nucleotides 1-8556):

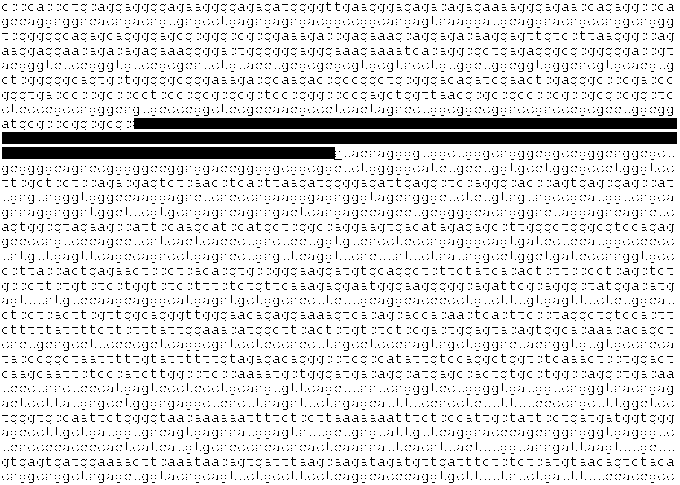

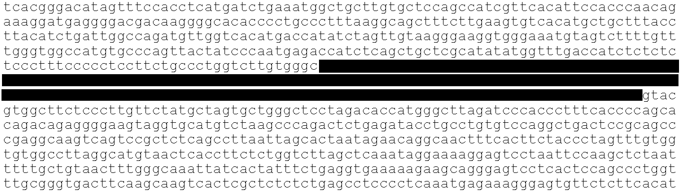

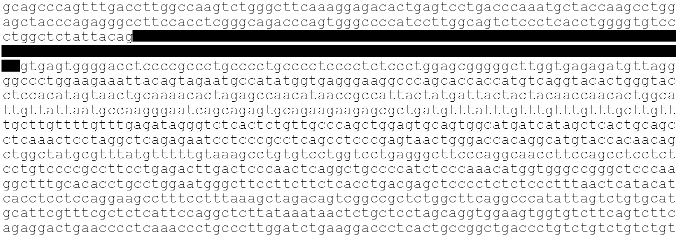

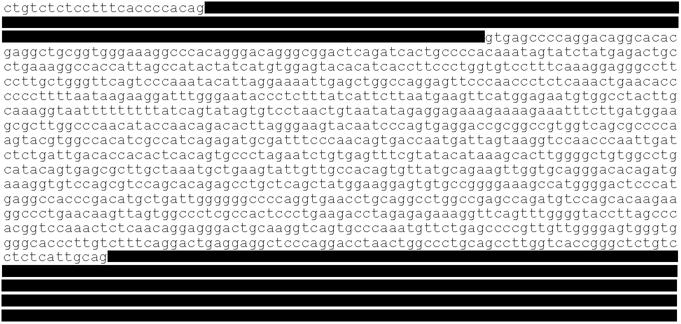

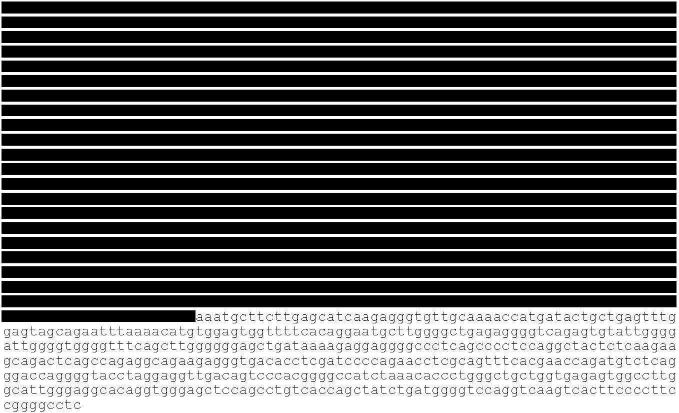

In yet other embodiments, the isolated human R-spondin 4 mutant molecule can comprise the nucleic acid sequence of SEQ ID NO: 21. For example, the nucleic acid sequence of this mutant contains a G>A mutation at the intron 1-exon 2 boundary (at about position 2887 of SEQ ID NO: 19; see reference IVS1−1G>A in FIG. 9D), which comprises SEQ ID NO: 21, and generates a splice site mutant predicted to result in aberrant splicing of RSPO4.

SEQ ID NO: 21 is the human mutant nucleic acid sequence corresponding to the R-spondin 4 G>A mutation at nucleic acid position 2887 (bold and underlined) of SEQ ID NO: 19 (nucleotides 1-8556):

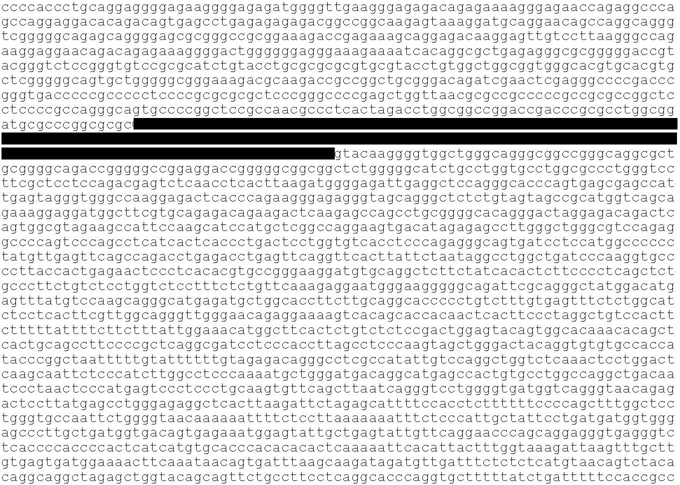

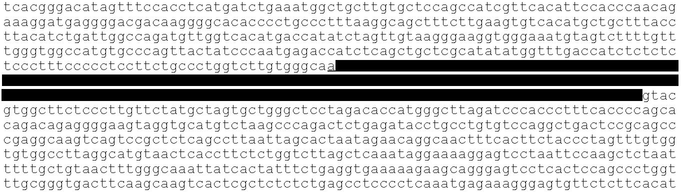

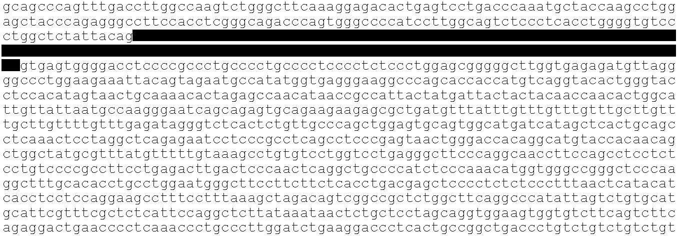

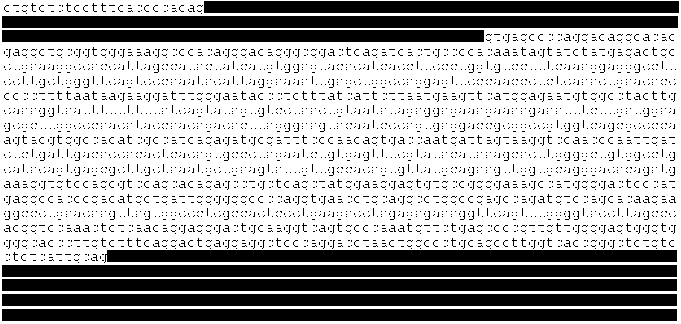

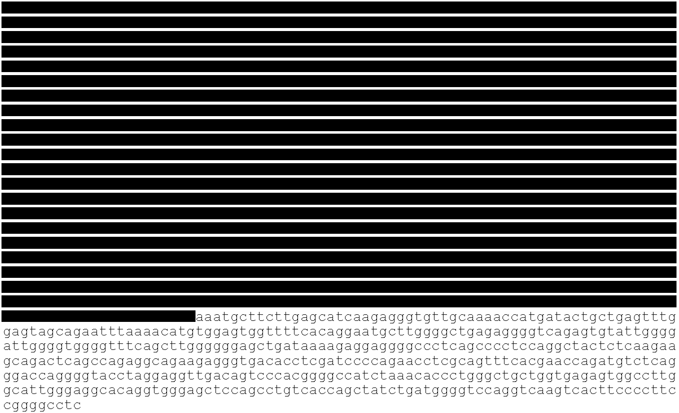

In other embodiments, human R-spondin 4 mutant molecules can arise from a G>A nucleic acid mutation at the intron 3-exon 3 boundary (see FIG. 9D), the intron 3-exon 4 boundary (see FIG. 9D), the intron 4-exon 4 boundary (see FIG. 9D), the intron 4-exon 5 boundary (see FIG. 9D), any of the mutants that give rise to RSPO4 splice variants described above, or a combination thereof, thus generating a splice site mutant predicted to result in aberrant splicing of RSPO4. The intron-exon boundaries are denoted as red nucleotides that precede or follow the shaded exon sequences (shadowed) in SEQ ID NO: 19.

Substantial changes in function or immunological identity are made by selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

Minor variations in the amino acid sequences of R-spondin 4 mutant proteins, variants, and fragments thereof can be encompassed by the present invention, providing that the variations in the amino acid sequence maintain at least 30%, 40%, 50%, 60%, 70%, 75%, 80%, 90%, 95%, or 99% of SEQ ID NO: 1. In particular, conservative amino acid replacements are contemplated. Conservative replacements are those that take place within a family of amino acids that are related in their side chains, wherein the interchangeability of residues have similar side chains.

Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) a group of amino acids having aliphatic-hydroxyl side chains, such as serine and threonine; (ii) a group of amino acids having amide-containing side chains, such as asparagine and glutamine; (iii) a group of amino acids having aliphatic side chains such as glycine, alanine, valine, leucine, and isoleucine; (iv) a group of amino acids having aromatic side chains, such as phenylalanine, tyrosine, and tryptophan; and (v) a group of amino acids having sulfur-containing side chains, such as cysteine and methionine. Particularly useful conservative amino acids substitution groups are: valine-leucine-isoleucine, phenylalanine-tyrosine, lysine-arginine, alanine valine, glutamic-aspartic, and asparagine-glutamine.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Substitutional or deletional mutagenesis can be employed to insert sites for N-glycosylation (Asn-X-Thr/Ser) or O-glycosylation (Ser or Thr). Deletions of cysteine or other labile residues also can be desirable. Deletions or substitutions of potential proteolysis sites, e.g. Arg, is accomplished for example by deleting one of the basic residues or substituting one by glutaminyl or histidyl residues.

The gene encoding a polypeptide or protein molecule of interest, (for example, R-spondin 4 polypeptide molecules or variants thereof, such as the R-spondin 4 mutants described above), can be cloned from either a genomic library or a cDNA according to standard protocols familiar to one skilled in the art. A cDNA, can be obtained by isolating total mRNA from a suitable cell line. Double stranded cDNAs can be prepared from the total mRNA using methods known in the art, and subsequently can be inserted into a suitable plasmid or bacteriophage vector. Genes can also be cloned using PCR techniques well established in the art. In one embodiment, a gene that encodes for example, a R-spondin 4 polypeptide molecule or a variant thereof, such as a R-spondin 4 mutant described above, can be cloned via PCR in accordance with the nucleotide sequence information provided by GenBank, and additionally by this invention. In a further embodiment, a DNA vector containing a cDNA encoding a R-spondin 4 polypeptide molecules or variants thereof, such as the R-spondin 4 mutants described above, can act as a template in PCR reactions wherein oligonucleotide primers designed to amplify a region of interest can be used, so as to obtain an isolated DNA fragment encompassing that region.

Paralogues are homologous genes in the same organism derived from a gene/chromosome/genome duplication, i.e. the common ancestor of the genes occurred since the last speciation event. Paralogues can be variants which have diverged within the same organism after a gene duplication event. Thus, there is a direct evolutionary relationship between homologues that may be reflected in structural and/or functional similarities. For example, R-spondin 4 orthologues may perform the same role in each organism in which they are found, while paralogues may perform functionally related (but distinct) roles within the same organism.

A peptidomimetic is a small protein-like chain designed to mimic a peptide that can arise from modification of an existing peptide in order to protect that molecule from enzyme degradation and increase its stability, and/or alter the molecule's properties (for example modifications that change the molecule's stability or biological activity). These modifications involve changes to the peptide that will not occur naturally (such as altered backbones and the incorporation of non-natural amino acids). Drug-like compounds may be able to be developed from existing peptides. A peptidomimetic can be a peptide, partial peptide or non-peptide molecule that mimics the tertiary binding structure or activity of a selected native peptide or protein functional domain (e.g., binding motif or active site). These peptide mimetics include recombinantly or chemically modified peptides, as well as non-peptide agents such as small molecule drug mimetics.

In one embodiment, the R-spondin 4 polypeptide molecule comprising SEQ ID NO: 1, variants, or fragments thereof, can be modified to produce peptide mimetics by replacement of one or more naturally occurring side chains of the 20 genetically encoded amino acids (or D amino acids) with other side chains, for instance with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4,5-, 6-, to 7-membered heterocyclics. For example, proline analogs can be made in which the ring size of the proline residue is changed from 5 members to 4, 6, or 7 members. Cyclic groups can be saturated or unsaturated, and if unsaturated, can be aromatic or non-aromatic. Heterocyclic groups can contain one or more nitrogen, oxygen, and/or sulphur heteroatoms. Examples of such groups include the furazanyl, ifuryl, imidazolidinyl imidazolyl, imidazolinyl, isothiazolyl, isoxazolyl, morpholinyl (e.g. morpholino), oxazolyl, piperazinyl (e.g. 1-piperazinyl), piperidyl (e.g. 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g. 1-pyrrolidinyl), pyrrolinyl, pyrrolyl, thiadiazolyl, thiazolyl, thienyl, thiomorpholinyl (e.g. thiomorpholino), and triazolyl. These heterocyclic groups can be substituted or unsubstituted. Where a group is substituted, the substituent can be alkyl, alkoxy, halogen, oxygen, or substituted or unsubstituted phenyl. Peptidomimetics may also have amino acid residues that have been chemically modified by phosphorylation, sulfonation, biotinylation, or the addition or removal of other moieties. For example, peptidomimetics can be designed and directed to amino acid sequences encoded by exon 1 (SEQ ID NO: 22), exon 2 (SEQ ID NO: 23), exon 3 (SEQ ID NO: 24), exon 4 (SEQ ID NO: 25), or exon 5 (SEQ ID NO: 26) of a R-spondin 4 polynucleotide molecule comprising SEQ ID NO: 1. For example, the molecule can be directed to a particular portion of R-spondin 4, such as a polypeptide molecule encoded by a portion of the nucleic acid sequence of exon 1, exon 2, exon 3, exon 4, or exon 5 of R-spondin 4, having SEQ ID NO: 27, 28, 29, 30 or 31, respectively.

SEQ ID NO: 22 is the human wild type amino acid sequence corresponding to exon 1 of R-spondin of SEQ ID NO: 1 (residues 1-27):

MRAPLCLLLL VAHAVDMLAL NRRKKQV

SEQ ID NO: 23 is the human wild type amino acid sequence corresponding to exon 2 of R-spondin of SEQ ID NO: 1 (residues 28-90):

GTG LGGNCTGCII CSEENGCSTC QQRLFLFIRR EGIRQYGKCL
HDCPPGYFGI RGQEVNRCKK

SEQ ID NO: 24 is the human wild type amino acid sequence corresponding to exon 3 of R-spondin of SEQ ID NO: 1 (residues 91-137):

CGATCESCFS QDFCIRCKRQ FYLYKGKCLP TCPPGTLAHQ
NTRECQG

SEQ ID NO: 25 is the human wild type amino acid sequence corresponding to exon 4 of R-spondin of SEQ ID NO: 1 (residues 138-199):

ECE LGPWGGWSPC THNGKTCGSA WGLESRVREA GRAGHEEAAT
CQVLSESRKC PIQRPCPGE

SEQ ID NO: 26 is the human wild type amino acid sequence corresponding to exon 5 of R-spondin of SEQ ID NO: 1 (residues 200-234):

R SPGQKKGRKD RRPRKDRKLD RRLDVRPRQP GLQP

SEQ ID NO: 27 is the human nucleic acid sequence corresponding to exon 1 of R-spondin 4 of SEQ ID NO: 2 (nucleotides 1-81):

ATGCGGGCGCCACTCTGCCTGCTCCTGCTCGTCGCCCACGCCGTGGACAT
GCTCGCCCTGAACCGAAGGAAGAAGCAAGTG

SEQ ID NO: 28 is the human nucleic acid sequence corresponding to exon 2 of R-spondin 4 of SEQ ID NO: 2 (nucleotides 82-270):

GGCACTGGCCTGGGGGGCAACTGCACAGGCTGTATCATCTGCTCAGAGGA
GAACGGCTGTTCCACCTGCCAGCAGAGGCTCTTCCTGTTCATCCGCCGGG
AAGGCATCCGCCAGTACGGCAAGTGCCTGCACGACTGTCCCCCTGGGTAC
TTCGGCATCCGCGGCCAGGAGGTCAACAGGTGCAAAAAG

SEQ ID NO: 29 is the human nucleic acid sequence corresponding to exon 3 of R-spondin 4 of SEQ ID NO: 2 (nucleotides 271-411):

TGTGGGGCCACTTGTGAGAGCTGCTTCAGCCAGGACTTCTGCATCCGGTG
CAAGAGGCAGTTTTACTTGTACAAGGGGAAGTGTCTGCCCACCTGCCCGC
CGGGCACTTTGGCCCACCAGAACACACGGGAGTGCCAGGGG

SEQ ID NO: 30 is the human nucleic acid sequence corresponding to exon 4 of R-spondin 4 of SEQ ID NO: 2 (nucleotides 412-597):

GAGTGTGAACTGGGTCCCTGGGGCGGCTGGAGCCCCTGCACACACAATGG
AAAGACCTGCGGCTCGGCTTGGGGCCTGGAGAGCCGGGTACGAGAGGCTG
GCCGGGCTGGGCATGAGGAGGCAGCCACCTGCCAGGTGCTTTCTGAGTCA
AGGAAATGTCCCATCCAGAGGCCCTGCCCAGGAGAG

SEQ ID NO: 31 is the human nucleic acid sequence corresponding to exon 5 of R-spondin 4 of SEQ ID NO: 2 (nucleotides 598-702):

AGGAGCCCCGGCCAGAAGAAGGGCAGGAAGGACCGGCGCCCACGCAAGGA
CAGGAAGCTGGACCGCAGGCTGGACGTGAGGCCGCGCCAGCCCGGCCTGC
AGCCC

A variety of techniques are available for constructing peptide mimetics with the same or similar desired biological activity as the corresponding native but with more favorable activity than the peptide with respect to solubility, stability, and/or susceptibility to hydrolysis or proteolysis (see, e.g., Morgan & Gainor, Ann. Rep. Med. Chem. 24, 243-252, 1989). Certain peptidomimetic compounds are based upon the amino acid sequence of the peptides of the invention. Peptidomimetic compounds can be synthetic compounds having a three-dimensional structure (i.e. a peptide motif) based upon the three-dimensional structure of a selected peptide. The peptide motif provides the peptidomimetic compound with the desired biological activity, wherein the binding activity of the mimetic compound is not substantially reduced, and is often the same as or greater than the activity of the native peptide on which the mimetic is modeled. Peptidomimetic compounds can have additional characteristics that enhance their therapeutic application, such as increased cell permeability, greater affinity and/or avidity and prolonged biological half-life.

Peptidomimetic design strategies are readily available in the art (see, e.g., Ripka & Rich, Curr. Op. Chem. Biol. 2, 441-452, 1998; Hruby et al., Curr. Op. Chem. Biol. 1, 114119, 1997; Hruby & Balse, Curr. Med. Chem. 9, 945-970, -2000). One class of peptidomimetics a backbone that is partially or completely non-peptide, but mimics the peptide backbone atom for a turn and comprises side groups that likewise mimic the functionality of the side groups of the native amino acid residues. Several types of chemical bonds, e.g. ester, thioester, thioamide, retroamide, reduced carbonyl, dimethylene and ketomethylene bonds, are known in the art to be generally useful substitutes for peptide bonds in the construction of protease-resistant peptidomimetics. Another class of peptidomimetics comprises a small non-peptide molecule that binds to another peptide or protein, but which is not necessarily a structural mimetic of the native peptide. Yet another class of peptidomimetics has arisen from combinatorial chemistry and the generation of massive chemical libraries. These generally comprise novel templates which, though structurally unrelated to the native peptide, possess necessary functional groups positioned on a nonpeptide scaffold to serve as topographical mimetics of the original peptide (Ripka & Rich, 1998, supra).

In a particular embodiment of the invention, the R-spondin 4 polypeptide molecule variants, fragments, or peptidomimetics are functional, in that they retain the nail growth stimulating activity of the wild type R-spondin 4 protein, and/or the Wnt activating function of wild type R-spondin 4 protein. In another embodiment, a R-spondin 4 polypeptide molecule variant or fragment thereof can comprise an amino acid sequence of exon 1 (SEQ ID NO: 22), exon 2 (SEQ ID NO: 23), exon 3 (SEQ ID NO: 24), exon 4 (SEQ ID NO: 25), and/or exon 5 (SEQ ID NO: 26) of RSPO4, wherein the RSPO4 comprises the amino acid sequence of SEQ ID NO: 1. In a further embodiment, a R-spondin 4 polypeptide molecule variant or fragment thereof can comprise an amino acid sequence encoded by the nucleic acid sequence of exon 1, exon 2, exon 3, exon 4, and/or exon 5 of a RSPO4 gene, wherein the RSPO4 gene comprises the nucleic acid sequence of SEQ ID NO: 2. For example, the amino acid sequence encoded by the nucleic acid sequences of exon 1, exon 2, exon 3, exon 4, or exon 5 of the RSPO4 gene (SEQ ID NOS: 27, 28, 29, 30, or 31, respectively) can be R-spondin 4 fragments. These fragments can also be used as competitive inhibitors of R-spondin 4 protein function.

R-Spondin 4 Production

One of skill in the art can readily produce, or isolate, the R-spondin 4 polypeptide molecules, or variants or fragments, thereof, for example using standard techniques that are well known to those of skill in the art. Any suitable technique can be used to produce or isolate the R-spondin 4 polypeptide molecules of the invention.

In one embodiment, the nucleic acid sequence encoding the R-spondin 4 polypeptide molecules is obtained, isolated or generated, and is then inserted into an expression vector containing a suitable promoter. The vector containing the R-spondin 4 molecule coding sequence under the control of a suitable promoter, can then be delivered to cells in which the R-spondin protein will be expressed. The R-spondin 4 polypeptide molecules may then be isolated from the cells, and optionally purified for use in the methods and compositions of the invention.

Expression of Recombinant R-Spondins in Host Cells

Standard molecular biology techniques can be used to obtain, isolate, or generate the nucleic acid encoding a R-spondin 4 protein, such as those techniques previously described.

The nucleic acid encoding the R-spondin 4 molecule can be inserted into any suitable expression vector using standard recombinant DNA and cloning techniques, such as those described in Sambrook et al. (1989) Molecular Cloning: A Laboratory Manual, 2nd Ed., Cold Spring Harbor Laboratory, Cold Spring Harbor, N.Y. (“Sambrook”).

Any expression vector capable of driving expression of the R-spondin 4 nucleic acid sequence in the desired cellular system can be used. An expression vector is a nucleic acid construct with a series of specified nucleic acid elements that permit transcription of a particular nucleic acid in a host cell. The expression vector can be a plasmid, virus, or a nucleic acid fragment. Typically, the expression vector includes a site for insertion of an exogenous coding sequence, such that insertion of the exogenous coding sequence will result in the coding sequence being operably linked to a promoter. To obtain high level expression of the R-spondin 4 nucleic acid sequence, it may be desirable to use an expression vector that contains a strong promoter to direct transcription, a transcription/translation terminator, and a ribosome binding site for translational initiation. Many suitable promoters and expression vectors are well known in the art, and described, e.g., in Sambrook. Furthermore, kits containing expression vectors and instructions for expressing proteins from these expression vectors, are available commercially. Expression vectors for expression of proteins in eukaryotic cells (such as mammalian cells, yeast, and insect cells), and prokaryotic cells, are well known in the art and are also commercially available.

Bacterial and Yeast Expression Systems: In bacterial systems, a number of expression vectors can be selected. For example, when a large quantity of R-spondin 4 molecules is needed for the induction of antibodies, vectors which direct high level expression of fusion proteins that are readily purified can be used. Non-limiting examples of such vectors include multifunctional E. coli cloning and expression vectors such as BLUESCRIPT (Stratagene). pIN vectors or pGEX vectors (Promega, Madison, Wis.) also can be used to express foreign polypeptide molecules as fusion proteins with glutathione S-transferase (GST). In general, such fusion proteins are soluble and can easily be purified from lysed cells by adsorption to glutathione-agarose beads followed by elution in the presence of free glutathione. Proteins made in such systems can be designed to include heparin, thrombin, or factor Xa protease cleavage sites so that the cloned polypeptide of interest can be released from the GST moiety at will.

Plant and Insect Expression Systems: If plant expression vectors are used, the expression of sequences encoding a R-spondin 4 molecule or a variant thereof, such as a mutant described above, can be driven by any of a number of promoters. For example, viral promoters such as the 35S and 19S promoters of CaMV can be used alone or in combination with the omega leader sequence from TMV. Alternatively, plant promoters such as the small subunit of RUBISCO or heat shock promoters can be used. These constructs can be introduced into plant cells by direct DNA transformation or by pathogen-mediated transfection.

An insect system also can be used to express R-spondin 4 molecules or a variant thereof, such as a R-spondin 4 mutant described above. For example, in one such system Autographa californica nuclear polyhedrosis virus (AcNPV) is used as a vector to express foreign genes in Spodoptera frugiperda cells or in Trichoplusia larvae. Sequences encoding, a R-spondin 4 molecule or a variant thereof, can be cloned into a non-essential region of the virus, such as the polyhedrin gene, and placed under control of the polyhedrin promoter. Successful insertion of R-spondin 4 or a variant thereof, will render the polyhedrin gene inactive and produce recombinant virus lacking coat protein. The recombinant viruses can then be used to infect S. frugiperda cells or Trichoplusia larvae in which R-spondin 4 or a variant thereof can be expressed.

Mammalian Expression Systems: An expression vector of the current invention can include a nucleotide sequence that encodes either a R-spondin 4 polypeptide molecule or a variant thereof, linked to at least one regulatory sequence in a manner allowing expression of the nucleotide sequence in a host cell.

A number of viral-based expression systems can be used to express a R-spondin 4 polypeptide molecule or a variant thereof, such as a R-spondin 4 mutant described above, in mammalian host cells. For example, if an adenovirus is used as an expression vector, sequences encoding a R-spondin 4 polypeptide molecule or a variant thereof, such as a R-spondin 4 mutant described above, can be ligated into an adenovirus transcription/translation complex comprising the late promoter and tripartite leader sequence. Insertion into a non-essential E1 or E3 region of the viral genome can be used to obtain a viable virus which is capable of expressing a R-spondin 4 polypeptide molecule or a variant thereof, such as a R-spondin 4 mutant described above, in infected host cells. Transcription enhancers, such as the Rous sarcoma virus (RSV) enhancer, can also be used to increase expression in mammalian host cells.

The expression vectors used will typically contain a suitable promoter capable of directing expression in the desired host cell type, such as in eukaryotic or prokaryotic cells. For example, expression vectors suitable for expression of the R-spondin 4 proteins of the invention in eukaryotic cells include, but are not limited to, the SV40 early promoter, SV40 later promoter, metallothionein promoter and the Rous sarcoma virus promoter. Regulatory sequences are well known to those skilled in the art, and can be selected to direct the expression of a protein or polypeptide molecule of interest (such as R-spondin 4 or a variant thereof, such as a R-spondin 4 mutant described above) in an appropriate host cell as described in Goeddel, Gene Expression Technology: Methods in Enzymology 185, Academic Press, San Diego, Calif. (1990). Non-limiting examples of regulatory sequences include: polyadenylation signals, promoters (such as CMV, ASV, SV40, or other viral promoters such as those derived from bovine papilloma, polyoma, and Adenovirus 2 viruses (Fiers, et al., 1973, Nature 273:113; Hager G L, et al., Curr Opin Genet Dev, 2002, 12(2):137-41) enhancers, and other expression control elements.

One skilled in the art also understands that enhancer regions, which are those sequences found upstream or downstream of the promoter region in non-coding DNA regions, are also important in optimizing expression. If needed, origins of replication from viral sources can be employed, such as if a prokaryotic host is utilized for introduction of plasmid DNA. However, in eukaryotic organisms, chromosome integration is a common mechanism for DNA replication.

The nucleic acid sequence encoding the R-spondin 4 polypeptide molecule may also be linked to a cleavable signal peptide sequence to promote secretion of the R-spondin 4 protein by the transformed cell. Suitable signal peptides include, but are not limited to, the signal peptides from tissue plasminogen activator, insulin, and neuron growth factor, and juvenile hormone esterase of Heliothis virescens.

A gene that encodes a selectable marker (for example, resistance to antibiotics or drugs, such as ampicillin, G418, and hygromycin) can be introduced into host cells along with the gene of interest in order to identify and select clones that stably express a gene encoding a protein of interest. The gene encoding a selectable marker can be introduced into a host cell on the same plasmid as the gene of interest or can be introduced on a separate plasmid. Cells containing the gene of interest can be identified by drug selection wherein cells that have incorporated the selectable marker gene will survive in the presence of the drug. Cells that have not incorporated the gene for the selectable marker die. Surviving cells can then be screened for the production of the desired protein molecule (for example, R-spondin 4 or a variant thereof, such as a R-spondin 4 mutant described above).

Some expression vector systems contain gene amplification sequences such as those from the thymidine kinase, hygromycin B phosphotransferase, and dihydrofolate reductase genes. Such expression vectors may be used to express the R-spondin 4 polypeptide molecules of the invention, and will typically result in a high level of expression of the R-spondin 4 proteins of the invention. Other high yield expression systems, not involving gene amplification are also known, such as the baculovirus expression system in which a baculovirus vector is used to drive expression of a recombinant protein in insect cells. Such baculovirus expression systems may be used to express the R-spondin 4 proteins of the invention, and will typically result a high level of expression of the R-spondin 4 proteins of the invention.

The R-spondin 4-encoding nucleic acids may optionally be fused to sequences encoding labels or tags, in order to produce a recombinant R-spondin 4 fusion protein containing the an N- or C-terminal tag, such as a GST tag, a LacZ tag, a FLAG tag, a c-myc tag, a His tag, a green fluorescent protein tag, and the like. Such tags and labels can be used to monitor expression of the R-spondin 4 proteins in host cells, and/or in methods for the isolation of the R-spondin 4 proteins.

Delivery of the Expression Vector to Host Cells

Standard transfection and transformation techniques may be used to deliver expression vectors containing R-spondin 4 coding sequences to the host cells in which the protein is to be expressed and produced. An exogenous nucleic acid can be introduced into a cell via a variety of techniques known in the art, such as lipofection, microinjection, calcium phosphate or calcium chloride precipitation, DEAE-dextrin-mediated transfection, or electroporation. Electroporation is carried out at approximate voltage and capacitance to result in entry of the DNA construct(s) into cells of interest (for example, keratinocytes). Other methods used to transfect cells can also include modified calcium phosphate precipitation, polybrene precipitation, liposome fusion, and receptor-mediated gene delivery. Transformation of eukaryotic and prokaryotic cells can be performed according to standard the techniques described in Morrison, J. Bact. 132:349 351 (1977); and Clark-Curtiss & Curtiss, Methods in Enzymology 101:347 362 (Wu et al., eds, 1983).

It is understood by those skilled in the art that for stable transfection of mammalian cells, a small fraction of cells can integrate introduced DNA into their genomes. The expression vector and transfection method utilized can be factors that contribute to a successful integration event. For stable amplification and expression of a desired protein, a vector containing DNA encoding a protein molecule of interest (for example, R-spondin 4 or a variant thereof, such as a R-spondin 4 mutant described above) is stably integrated into the genome of eukaryotic cells (for example mammalian cells), resulting in the stable expression of transfected genes. An exogenous nucleic acid sequence can be introduced into a cell (such as a mammalian cell, either primary or secondary cell) by homologous recombination as disclosed in U.S. Pat. No. 5,641,670, the contents of which are herein incorporated by reference.

A eukaryotic expression vector can be used to transfect cells in order to produce proteins (for example, R-spondin 4 or a variant thereof, such as a R-spondin 4 mutant described above) encoded by nucleotide sequences of the vector. Mammalian cells (such as isolated cells from the epidermis) can contain an expression vector (for example, one that contains a gene encoding R-spondin 4 or a variant thereof, such as a R-spondin 4 mutant described above) via introducing the expression vector into an appropriate host cell via methods known in the art.

A host cell strain can be chosen for its ability to modulate the expression of the inserted sequences or to process the expressed a R-spondin 4 polypeptide molecule or a variant thereof, such as a R-spondin 4 mutant described above, in the desired fashion. Such modifications of the polypeptide include, but are not limited to, acetylation, carboxylation, glycosylation, phosphorylation, lipidation, and acylation. Post-translational processing which cleaves a “prepro” form of the polypeptide also can be used to facilitate correct insertion, folding and/or function. Different host cells which have specific cellular machinery and characteristic mechanisms for post-translational activities (e.g., CHO, HeLa, MDCK, HEK293, and W138), are available from the American Type Culture Collection (ATCC; 10801 University Boulevard, Manassas, Va. 20110-2209) and can be chosen to ensure the correct modification and processing of the foreign protein.

Cells to be genetically engineered can be primary and secondary cells that can be obtained from various tissues, and include cell types which can be maintained and propagated in culture. Non-limiting examples of primary and secondary cells include epithelial cells (for example, dermal papilla cells), neural cells, endothelial cells, glial cells, fibroblasts, muscle cells (such as myoblasts) keratinocytes, formed elements of the blood (e.g., lymphocytes, bone marrow cells), and precursors of these somatic cell types.

Vertebrate tissue can be obtained by methods known to one skilled in the art, such a punch biopsy or other surgical methods of obtaining a tissue source of the primary cell type of interest. In one embodiment, a punch biopsy or removal can be used to obtain a source of keratinocytes, fibroblasts, endothelial cells, or mesenchymal cells. A mixture of primary cells can be obtained from the tissue, using methods readily practiced in the art, such as explanting or enzymatic digestion (for examples using enzymes such as pronase, trypsin, collagenase, elastase dispase, and chymotrypsin). Biopsy methods have also been described in United States Patent Application Publication No. 2004/0057937 and PCT application publication WO 2001/32840, and are hereby incorporated by reference.

Primary cells can be acquired from the individual to whom the genetically engineered primary or secondary cells are administered. However, primary cells can also be obtained from a donor, other than the recipient, of the same species. The cells may also be obtained from another species (for example, rabbit, cat, mouse, rat, sheep, goat, dog, horse, cow, bird, or pig). Primary cells can also include cells from an isolated vertebrate tissue source grown attached to a tissue culture substrate (for example, flask or dish) or grown in a suspension; cells present in an explant derived from tissue; both of the aforementioned cell types plated for the first time; and cell culture suspensions derived from these plated cells. Secondary cells can be plated primary cells that are removed from the culture substrate and replated, or passaged, in addition to cells from the subsequent passages. Secondary cells can be passaged one or more times. These primary or secondary cells can contain expression vectors having a gene that encodes a protein molecule of interest (for example, R-spondin 4 or a variant thereof, such as a R-spondin 4 mutant described above).

Cell Culturing

Various culturing parameters can be used with respect to the host cell being cultured. Appropriate culture conditions for mammalian cells are well known in the art (Cleveland W L, et al., J Immunol Methods, 1983, 56(2): 221-234) or can be determined by the skilled artisan (see, for example, Animal Cell Culture: A Practical Approach 2nd Ed., Rickwood, D. and Hames, B. D., eds. (Oxford University Press: New York, 1992)). Cell culturing conditions can vary according to the particular host cell selected. Commercially available medium can be utilized. Non-limiting examples of medium include, for example, Minimal Essential Medium (MEM, Sigma, St. Louis, Mo.); Dulbecco's Modified Eagles Medium (DMEM, Sigma); Ham's F10 Medium (Sigma); HyClone cell culture medium (HyClone, Logan, Utah); RPMI-1640 Medium (Sigma); and chemically-defined (CD) media, which are formulated for particular cell types, e.g. CD-CHO Medium (Invitrogen, Carlsbad, Calif.).

The media described above can be supplemented as necessary with supplementary components or ingredients, including optional components, in appropriate concentrations or amounts, as necessary or desired. Cell medium solutions provide at least one component from one or more of the following categories: (1) an energy source, usually in the form of a carbohydrate such as glucose; (2) all essential amino acids, and usually the basic set of twenty amino acids plus cysteine; (3) vitamins and/or other organic compounds required at low concentrations; (4) free fatty acids or lipids, for example linoleic acid; and (5) trace elements, where trace elements are defined as inorganic compounds or naturally occurring elements that are typically required at very low concentrations, usually in the micromolar range.

The medium also can be supplemented electively with one or more components from any of the following categories: (1) salts, for example, magnesium, calcium, and phosphate; (2) hormones and other growth factors such as, serum, insulin, transferrin, and epidermal growth factor; (3) protein and tissue hydrolysates, for example peptone or peptone mixtures which can be obtained from purified gelatin, plant material, or animal byproducts; (4) nucleosides and bases such as, adenosine, thymidine, and hypoxanthine; (5) buffers, such as HEPES; (6) antibiotics, such as gentamycin or ampicillin; (7) cell protective agents, for example pluronic polyol; and (8) galactose. In one embodiment, soluble factors can be added to the culturing medium.

Isolation and Purification of the R-spondin 4 Polypeptide Molecules

After the recombinant R-spondin 4 proteins of the invention have been expressed in host cells, they can be then be purified using standard protein purification techniques, such as those described in Guide to Protein Purification, in Methods in Enzymology, vol. 182 (Deutscher, ed., 1990)). A R-spondin 4 polypeptide molecule or a variant thereof, such as a R-spondin 4 mutant described above, can be obtained, for example, by purification from human cells, via expression of a R-spondin 4 molecule or a variant thereof, polynucleotides, or by direct chemical synthesis.

Protein Purification: A R-spondin 4 polypeptide molecule or a variant thereof, such as a R-spondin 4 mutant described above, can be purified from any human cell which expresses the receptor, including those which have been transfected with expression constructs which express a R-spondin 4 molecule or a variant thereof. A purified R-spondin 4 molecule or a R-spondin 4 mutant described above can be separated from other compounds which normally associate with R-spondin 4 or a variant thereof, in the cell, such as certain proteins, carbohydrates, or lipids, using methods well-known in the art. Such methods include, but are not limited to, size exclusion chromatography, ammonium sulfate fractionation, ion exchange chromatography, affinity chromatography, and preparative gel electrophoresis.

Detecting Polypeptide Expression: Although the presence of marker gene expression suggests that a polynucleotide of R-spondin 4 or a variant thereof is also present, its presence and expression may need to be confirmed. For example, if a sequence encoding a R-spondin 4 polypeptide molecule or a variant thereof, such as a R-spondin 4 mutant described above, is inserted within a marker gene sequence, transformed cells containing sequences which encode a R-spondin 4 polypeptide molecule or such a variant previously described, can be identified by the absence of marker gene function. Alternatively, a marker gene can be placed in tandem with a sequence encoding a R-spondin 4 polypeptide molecule or a variant thereof, such as a R-spondin 4 mutant described above, under the control of a single promoter. Expression of the marker gene in response to induction or selection usually indicates expression of a polynucleotide of R-spondin 4 or a R-spondin 4 mutant described above.

Alternatively, host cells which contain a polynucleotide of R-spondin 4 or a variant thereof, such as a R-spondin 4 mutant described above, and which express R-spondin 4 or a variant thereof, can be identified by a variety of procedures known to those of skill in the art. These procedures include, but are not limited to, DNA-DNA or DNA-RNA hybridizations and protein bioassay or immunoassay techniques which include membrane, solution, or chip-based technologies for the detection and/or quantification of nucleic acid or protein. For example, the presence of a polynucleotide sequence encoding R-spondin 4 or a variant thereof, such as a R-spondin 4 mutant described above, can be detected by DNA-DNA or DNA-RNA hybridization or amplification using probes or fragments or fragments of polynucleotides encoding R-spondin 4 or a variant thereof. Nucleic acid amplification-based assays involve the use of oligonucleotides selected from sequences encoding a R-spondin 4 polypeptide molecule or a R-spondin 4 mutant described above, to detect transformants which contain a polynucleotide of R-spondin 4 or a variant thereof.

A variety of protocols are known in the art for detecting and measuring the expression of R-spondin 4 or a variant thereof, such as a R-spondin 4 mutant described above, using either polyclonal or monoclonal antibodies specific for the polypeptide. Examples include enzyme-linked immunosorbent assay (ELISA), radioimmunoassay (RIA), and fluorescence activated cell sorting (FACS). A two-site, monoclonal-based immunoassay using monoclonal antibodies reactive to two non-interfering epitopes on a R-spondin 4 polypeptide molecule or a variant thereof can be used, or a competitive binding assay can be employed.

A wide variety of labels and conjugation techniques are known by those skilled in the art and can be used in various nucleic acid and amino acid assays. Methods for producing labeled hybridization or PCR probes for detecting sequences related to polynucleotides encoding R-spondin 4 or a variant thereof, such as a R-spondin 4 mutant described above, include oligolabeling, nick translation, end-labeling, or PCR amplification using a labeled nucleotide. Alternatively, sequences encoding a R-spondin 4 polypeptide molecule or a R-spondin 4 mutant described above, can be cloned into a vector for the production of an mRNA probe. Such vectors are known in the art, are commercially available, and can be used to synthesize RNA probes in vitro by addition of labeled nucleotides and an appropriate RNA polymerase such as T7, T3, or SP6. These procedures can be conducted using a variety of commercially available kits (Amersham Pharmacia Biotech, Promega, and US Biochemical). Suitable reporter molecules or labels which can be used for ease of detection include radionuclides, enzymes, and fluorescent, chemiluminescent, or chromogenic agents, as well as substrates, cofactors, inhibitors, magnetic particles, and the like.

Expression and Purification of Polypeptides: Host cells transformed with polynucleotides of R-spondin 4 or a variant thereof, such as a R-spondin 4 mutant described above, can be cultured under conditions suitable for the expression and recovery of the protein from cell culture. The polypeptide produced by a transformed cell can be secreted or contained intracellularly depending on the sequence and/or the vector used. As will be understood by those of skill in the art, expression vectors containing polynucleotides of R-spondin 4 or a R-spondin 4 mutant described above, can be designed to contain signal sequences which direct secretion of soluble R-spondin 4 polypeptide molecules or a variant thereof, through a prokaryotic or eukaryotic cell membrane or which direct the membrane insertion of membrane-bound R-spondin 4 polypeptide molecule or a variant thereof.

As discussed above, other constructions can be used to join a sequence encoding a R-spondin 4 polypeptide molecule or a variant thereof, such as a R-spondin 4 mutant described above, to a nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). Including cleavable linker sequences (i.e., those specific for Factor Xa or enterokinase (Invitrogen, San Diego, Calif.)) between the purification domain and a R-spondin 4 polypeptide molecule or a variant thereof also can be used to facilitate purification. One such expression vector provides for expression of a fusion protein containing R-spondin 4 or a variant thereof and 6 histidine residues preceding a thioredoxin or an enterokinase cleavage site. The histidine residues facilitate purification by immobilized metal ion affinity chromatography, while the enterokinase cleavage site provides a means for purifying the R-spondin 4 polypeptide molecule or a variant thereof from the fusion protein.

Chemical Synthesis of Polypeptides

The above describes the expression and isolation of recombinant R-spondin 4 proteins using a cellular expression system. However, various other methods can be used to obtain or produce the R-spondin 4 proteins of the invention. For example, in one embodiment, the R-spondin 4 proteins may be synthetically generated, expressed using an in vitro transcription and translation system, or may be isolated from a natural source, such as from tissues or bodily fluids of an animal that expresses R-spondin 4 proteins. Any suitable technique may be used.

Sequences encoding a R-spondin 4 polypeptide molecule or a variant thereof, such as a R-spondin 4 mutant described above, can be synthesized, in whole or in part, using chemical methods well known in the art. Alternatively, a R-spondin 4 molecule or a variant thereof can be produced using chemical methods to synthesize its amino acid sequence, such as by direct peptide synthesis using solid-phase techniques. Protein synthesis can either be performed using manual techniques or by automation. Automated synthesis can be achieved, for example, using Applied Biosystems 431A Peptide Synthesizer (Perkin Elmer). Optionally, fragments of R-spondin 4 molecules or variants thereof, such as a R-spondin 4 mutant described above, can be separately synthesized and combined using chemical methods to produce a full-length molecule.

The newly synthesized peptide can be substantially purified via high performance liquid chromatography (HPLC). The composition of a synthetic R-spondin 4 molecule or a variant thereof can be confirmed by amino acid analysis or sequencing. Additionally, any portion of the amino acid sequence of R-spondin 4 or a R-spondin 4 mutant described above, or fragments comprising R-spondin 4 exon 1, exon 2, exon 3, and the like, can be altered during direct synthesis and/or combined using chemical methods with sequences from other proteins to produce a variant polypeptide or a fusion protein. For example, such fragments can function as competitive inhibitors or can be used to rescue a mutant R-spondin 4 phenotype (for example, a keratin-related abnormality such as nail, hook, or claw hypoplasia).

Identifying R-spondin Modulating Compounds

The invention provides methods for identifying compounds which can be used for controlling and/or regulating nail growth and strength in a subject. In addition, the invention provides methods for identifying compounds which can be used for the treatment of a claw, nail, or hoof keratin-related abnormality in a subject. In one embodiment, the abnormality can be an inherited abnormality. Non-limiting examples of inherited abnormalities include: anonychia congenita, hyponychia congenita, Cooks syndrome, nail patella syndrome, ectodermal dysplasias, and epidermolysis bullosa. The claw, nail, or hoof abnormality can also be caused by an infection (such as a bacterium, a fungus, a yeast, a mold, a virus, or any combination thereof), or can be characterized by slow or absent growth or repair of the nail, hoof, or claw.

The methods can comprise the identification of test compounds or agents (e.g., peptides, fragments, peptidomimetics, small molecules, or other molecules) that can bind to a R-spondin 4 polypeptide molecule and/or have a stimulatory or inhibitory effect on the biological activity of R-spondin 4 or its expression, and subsequently determining whether these compounds can regulate nail, hoof, or claw growth in a subject (i.e., examining an increase or reduction in nail, claw, or hoof growth and/or strength).

Knowledge of the primary sequence of a molecule of interest, such as a R-spondin 4 polypeptide or a variant thereof, and the similarity of that sequence with proteins of known function (such as other R-spondin proteins within the organism or in other species), can provide an initial clue as to the inhibitors or antagonists of the protein of interest. Identification and screening of antagonists is further facilitated by determining structural features of the protein, e.g., using X-ray crystallography, neutron diffraction, nuclear magnetic resonance spectrometry, and other techniques for structure determination. These techniques provide for the rational design or identification of agonists and antagonists.

Test compounds, such as R-spondin 4 modulating compounds, can be screened from large libraries of synthetic or natural compounds (see Wang et al., (2007) Curr Med Chem, 14(2):133-55; Mannhold (2006) Curr Top Med Chem, 6 (10):1031-47; and Hensen (2006) Curr Med Chem 13(4):361-76). Numerous means are currently used for random and directed synthesis of saccharide, peptide, and nucleic acid based compounds. Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (N.C.), or are readily producible. Additionally, natural and synthetically produced libraries and compounds are readily modified through conventional chemical, physical, and biochemical means (Blondelle et al., (1996) Tib Tech 14:60).

Computer modeling and searching technologies permit the identification of compounds, or the improvement of already identified compounds, that can modulate R-spondin 4 expression or activity. Upon identifying such a compound or composition, the active sites or regions of a R-spondin 4 polypeptide molecule can be subsequently identified via examining the sites as to which the compounds bind. These active sites may be ligand binding sites and can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the active site by finding where on the factor the complexed ligand is found.

Screening the libraries can be accomplished by any variety of commonly known methods. See, for example, the following references, which disclose screening of peptide libraries: Parmley and Smith, (1989) Adv. Exp. Med. Biol. 251:215-218; Scott and Smith, (1990) Science 249:386-390; Fowlkes et al., (1992) BioTechniques 13:422-427; Oldenburg et al., (1992) Proc. Natl. Acad. Sci. USA 89:5393-5397; Yu et al., (1994) Cell 76:933-945; Staudt et al., (1988) Science 241:577-580; Bock et al., (1992) Nature 355:564-566; Tuerk et al., (1992) Proc. Natl. Acad. Sci. USA 89:6988-6992; Ellington et al., (1992) Nature 355:850-852; U.S. Pat. Nos. 5,096,815; 5,223,409; and 5,198,346, all to Ladner et al.; Rebar et al., (1993) Science 263:671-673; and PCT Publication WO 94/18318.

The three dimensional geometric structure of an active site, for example that of a R-spondin 4 polypeptide molecule or a variant thereof, can be determined by known methods in the art, such as X-ray crystallography, which can determine a complete molecular structure. Solid or liquid phase NMR can be used to determine certain intramolecular distances. Any other experimental method of structure determination can be used to obtain partial or complete geometric structures. The geometric structures may be measured with a complexed ligand, natural or artificial, which may increase the accuracy of the active site structure determined.

One of skill in the art will be familiar with methods for predicting the effect on protein conformation of a change in protein sequence, and can thus design a variant which functions as an antagonist according to known methods. One example of such a method is described by Dahiyat and Mayo in Science (1997) 278:82 87, which describes the design of proteins de novo. The method can be applied to a known protein to vary only a portion of the polypeptide sequence. By applying the computational methods of Dahiyat and Mayo, R-spondin 4 modulating compounds confined to regions which bind the active site of a R-spondin 4 polypeptide molecule can be proposed and tested to determine whether the compound or the variant retains a desired conformation. Similarly, Blake (U.S. Pat. No. 5,565,325) teaches the use of known ligand structures to predict and synthesize variants with similar or modified function.

The present invention is also directed to methods for inhibiting or decreasing the growth of, or weakening, a keratin-containing limb appendage, such as a nail, hoof, or claw in a subject, comprising administering to the subject a composition comprising an agent inhibits or decreases the expression of an R-spondin 4 polypeptide molecule. In one embodiment, the agent can inhibit or decrease expression of R-spondin 4 via RNA interference. Thus, in certain aspects, the invention is directed to “interfering RNA” or “iRNA” molecules which target nucleic acids encoding R-spondin 4 polypeptide molecules, to compositions containing such iRNA molecules, and to methods of inhibiting or decreasing the growth of, or weakening, a keratin-containing limb appendage, such as a nail, hoof, or claw in a subject, comprising administering to the subject a composition comprising an iRNA molecule.

An iRNA agent is an RNA agent, which can down-regulate the expression of a target gene, e.g. a gene encoding a R-spondin 4 protein. An iRNA agent may act by one or more of a number of mechanisms, including post-transcriptional cleavage of a target mRNA sometimes referred to in the art as RNAi, or pre-transcriptional or pre-translational mechanisms.

An iRNA agent can be a double stranded (ds) iRNA agent. A ds iRNA agent is an iRNA agent which includes more than one, and in certain embodiments two, strands in which interchain hybridization can form a region of duplex structure. A strand can be a contiguous sequence of nucleotides (including non-naturally occurring or modified nucleotides). The two or more strands may be, or each form a part of, separate molecules, or they may be covalently interconnected, e.g. by a linker, e.g. a polyethyleneglycol linker, to form but one molecule. At least one strand can include a region which is sufficiently complementary to a target RNA. Such strand is the antisense strand. A second strand comprised in the dsRNA agent which comprises a region complementary to the antisense strand is referred to as the sense strand. However, a ds iRNA agent can also be formed from a single RNA molecule which is, at least partly; self-complementary, forming, e.g., a hairpin or panhandle structure, including a duplex region. In such case, the term “strand” can refer to one of the regions of the RNA molecule that is complementary to another region of the same RNA molecule.

Although, in animal cells, long ds iRNA agents can induce the interferon response, which is frequently deleterious, short ds iRNA agents do not trigger the interferon response, at least not to an extent that is deleterious to the cell and/or host. The iRNA agents of the present invention include molecules that are sufficiently short that they do not trigger a deleterious interferon response in mammalian cells. Thus, the administration of a composition of an iRNA agent (e.g., formulated as described herein) to an animal can be used to block expression of the R-spondin 4 gene while circumventing a deleterious interferon response.

Molecules that are short enough that they do not trigger a deleterious interferon response are termed siRNA agents or siRNAs herein. “siRNA agent” or “siRNA” as used herein, refers to an iRNA agent, e.g., a ds iRNA agent, that is sufficiently short that it does not induce a deleterious interferon response in a human cell, e.g., it has a duplexed region of less than about 30 nucleotide pairs.

iRNA agents as described herein, including ds iRNA agents and siRNA agents, can mediate silencing of a gene, e.g., by RNA degradation. For convenience, such RNA is also referred to herein as the RNA to be silenced. Such a gene is also referred to as a target gene. In certain embodiments, the RNA to be silenced is a gene product of a picornavirus gene, for example but not limited to viral VP1, 2, 3, and 4 gene product. As used herein, the phrase “mediates RNAi” refers to the ability of an agent to silence, in a sequence specific manner, a target gene. “Silencing a target gene” means the process whereby a cell containing and/or secreting a certain product of the target gene when not in contact with the agent, will contain and/or secrete at least 10%, 20%, 30%, 40%, 50%, 60%, 70%, 80%, or 90% less of such gene product when contacted with the agent, as compared to a similar cell which has not been contacted with the agent. Such product of the target gene can, for example, be a messenger RNA (mRNA), a protein, or a regulatory element.

siRNA comprises a double stranded structure typically containing 15 to 50 base pairs and preferably 21 to 25 base pairs and having a nucleotide sequence identical or nearly identical to an expressed target gene or RNA within the cell. Antisense polynucleotides include, but are not limited to: morpholinos, 2′-O-methyl polynucleotides, DNA, RNA and the like. RNA polymerase III transcribed DNAs contain promoters, such as the U6 promoter. These DNAs can be transcribed to produce small hairpin RNAs in the cell that can function as siRNA or linear RNAs that can function as antisense RNA. The inhibitor may be polymerized in vitro, recombinant RNA, contain chimeric sequences, or derivatives of these groups. The inhibitor may contain ribonucleotides, deoxyribonucleotides, synthetic nucleotides, or any suitable combination such that the target RNA and/or gene is inhibited. In addition, these forms of nucleic acid may be single, double, triple, or quadruple stranded. (see for example Bass (2001) Nature, 411, 428 429; Elbashir et al., (2001) Nature, 411, 494 498; and PCT Publication Nos. WO 00/44895, WO 01/36646, WO 99/32619, WO 00/01846, WO 01/29058, WO 99/07409, WO 00/44914). For example, siRNA molecule can be directed to a particular portion of R-spondin 4 (such as exon 1, exon 2, exon 3, exon 4, or exon 5 of the RSPO4 gene (SEQ ID NOS: 27, 28, 29, 30, or 31, respectively)).

In another embodiment, the agent that inhibits or decreases the expression of the R-spondin 4 polypeptide molecule via RNA interference can be antisense molecules which target nucleic acids encoding R-spondin 4 polypeptide molecules. Antisense oligonucleotides, including antisense DNA, RNA, and DNA/RNA molecules, act to directly block the translation of mRNA by binding to targeted mRNA and preventing protein translation. For example, antisense oligonucleotides of at least about 15 bases and complementary to unique regions of the DNA sequence encoding a neuraminidase polypeptide can be synthesized, e.g., by conventional phosphodiester techniques (Dallas et al., (2006) Med. Sci. Monit. 12(4):RA67-74; Kalota et al., (2006) Handb. Exp. Pharmacol. 173:173-96; Lutzelburger et al., (2006) Handb. Exp. Pharmacol. 173:243-59). For example, the antisense RNA can be directed to a particular portion of R-spondin 4 (such as exon 1, exon 2, exon 3, exon 4, or exon 5 of the RSPO4 gene (SEQ ID NOS: 27, 28, 29, 30, or 31, respectively)).

In a further embodiment, the agent that inhibits or decreases expression of R-spondin 4 via RNA interference can be an inhibitory transcription factor. The inhibitory transcription factor can be a repressor protein coded by the repressor mRNA transcript and may be capable of directly interacting with the regulatory sequences of the repressed gene, whether endogenous or engineered, as known in the art, or may indirectly interact with other biomolecules present in the cell to repress the repressed gene. For further reference, see Latchman, D (1996) Int J Biochem Cell Biol. 28(9):965-74, which is hereby incorporated by reference.

In other aspects, the invention is directed to isolated nucleic acid sequences such as primers and probes, comprising nucleic acid sequences derived from any one of SEQ ID NOS: 2, 7-12, 15-21, or 27-31, or those sequences listed in Table 2. Such primers and/or probes may be useful for detecting the presence of the picornaviruses of the invention, for example in samples of bodily fluids such as blood, saliva, or urine from a subject, and thus may be useful in the diagnosis of picornavirus infection. Such probes can detect polynucleotides of SEQ ID NOS: 2, 7-12, 15-21, or 27-31 in samples which comprise picornaviruses represented by SEQ ID NOS: 2, 7-12, 15-21, or 27-31. The isolated nucleic acids which can be used as primer and/probes are of sufficient length to allow hybridization with, i.e. formation of duplex with a corresponding target nucleic acid sequence, a nucleic acid sequences of any one of SEQ ID NOS: 2, 7-12, 15-21, or 27-31, or a variant thereof.

The isolated nucleic acid of the invention which can be used as primers and/or probes can comprise about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 consecutive nucleotides from any one of SEQ ID NOS: 2, 7-12, 15-21, or 27-31, or sequences complementary to any one of SEQ ID NOS: 2, 7-12, 15-21, or 27-31. In one embodiment, the number of consecutive nucleotides that can be used as primers and/or probes can comprise from about 4 to about 40 nucleotides, from about 5 to about 35 nucleotides, from about 6 to about 30 nucleotides, from about 7 to about 25 nucleotides, from about 8 to about 20 nucleotides, from about 9 to about 15 nucleotides, or any range therein, wherein the consecutive nucleotides are obtained from any one of SEQ ID NOS: 2, 7-12, 15-21, or 27-31, or sequences complementary to any one of SEQ ID NOS: 2, 7-12, 15-21, or 27-31.

The isolated nucleic acid of the invention which can be used as primers and/or probes can comprise from about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 and up to about 20, 25, 30, 35, 40, 45, 50, 55, 60, 65, 70, 75, 80, 85, 90, 95 and 100 consecutive nucleotides from any one of SEQ ID NOS: 2, 7-12, 15-21, or 27-31, or sequences complementary to any one of SEQ ID NOS: 2, 7-12, 15-21, or 27-31. In another embodiment, the number of consecutive nucleotides that can be used as primers and/or probes can comprise from about 4 to about 100 nucleotides, from about 5 to about 95 nucleotides, from about 6 to about 90 nucleotides, from about 7 to about 85 nucleotides, from about 8 to about 80 nucleotides, from about 9 to about 75 nucleotides, from about 10 to about 70 nucleotides, from about 11 to about 65 nucleotides, from about 12 to about 60 nucleotides, from about 13 to about 55 nucleotides, from about 14 to about 50 nucleotides, from about 15 to about 45 nucleotides, from about 16 to about 40 nucleotides, from about 17 to about 35 nucleotides, from about 18 to about 30 nucleotides, from about 19 to about 25 nucleotides, or any range therein, wherein the consecutive nucleotides are obtained from any one of SEQ ID NOS: 2, 7-12, 15-21, or 27-31, or sequences complementary to any one of SEQ ID NOS: 2, 7-12, 15-21, or 27-31. The invention is also directed to primer and/or probes which can be labeled by any suitable molecule and/or label known in the art, for example but not limited to fluorescent tags suitable for use in Real Time PCR amplification, for example TaqMan™, cybergreen, TAMRA and/or FAM probes; radiolabels, and so forth. In certain embodiments, the oligonucleotide primers and/or probe further comprises a detectable non-isotopic label selected from the group consisting of: a fluorescent molecule, a chemiluminescent molecule, an enzyme, a cofactor, an enzyme substrate, and a hapten.

In certain aspects, the invention is directed to primer sets comprising isolated nucleic acids as described herein, which primer set are suitable for amplification of nucleic acids from samples which comprises picornaviruses represented by any one of SEQ ID NOS: 2, 7-12, 15-21, or 27-31, or variants thereof. Primer sets can comprise any suitable combination of primers which would allow amplification of a target nucleic acid sequences in a sample which comprises picornaviruses represented by any one of SEQ ID NOS: 2, 7-12, 15-21, or 27-31, or variants thereof. Amplification can be performed by any suitable method known in the art, for example but not limited to PCR, RT-PCR, transcription mediated amplification (TMA).

Hybridization conditions: As used herein, the phrase “stringent hybridization conditions” refers to conditions under which a probe, primer or oligonucleotide will hybridize to its target sequence, and can hybridize, for example but not limited to, variants of the disclosed polynucleotide sequences, including allelic or splice variants, or sequences that encode orthologs or paralogs of presently disclosed polypeptides. The precise conditions for stringent hybridization are typically sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Nucleic acid hybridization methods are disclosed in detail by Kashima et al. (1985) Nature 313:402-404, and Sambrook et al. (1989) Molecular Cloning: A Laboratory Manual, 2nd Ed., Cold Spring Harbor Laboratory, Cold Spring Harbor, N.Y. (“Sambrook”); and by Haymes et al., “Nucleic Acid Hybridization: A Practical Approach”, IRL Press, Washington, D.C. (1985), which references are incorporated herein by reference.

In general, stringency is determined by the temperature, ionic strength, and concentration of denaturing agents (e.g., formamide) used in a hybridization and washing procedure. The degree to which two nucleic acids hybridize under various conditions of stringency is correlated with the extent of their similarity. Numerous variations are possible in the conditions and means by which nucleic acid hybridization can be performed to isolate nucleic sequences having similarity to the nucleic acid sequences known in the art and are not limited to those explicitly disclosed herein. Such an approach may be used to isolate polynucleotide sequences having various degrees of similarity with disclosed nucleic acid sequences, such as, for example, nucleic acid sequences having 60% identity, or about 70% identity, or about 80% or greater identity with disclosed nucleic acid sequences.

Stringent conditions are known to those skilled in the art and can be found in Current Protocols In Molecular Biology, John Wiley & Sons, N.Y. (1989), 6.3.1-6.3.6. In certain embodiments, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6× sodium chloride/sodium citrate (SSC), 50 mM Tris-HCl (pH 7.5), 1 nM EDTA, 0.02% PVP, 0.02% Ficoll, 0.02% BSA, and 500 mg/ml denatured salmon sperm DNA at 65° C. This hybridization is followed by one or more washes in 0.2×SSC, 0.01% BSA at 50° C. Another non-limiting example of stringent hybridization conditions are hybridization in 6× sodium chloride/sodium citrate (SSC) at about 45° C., followed by one or more washes in 0.2×SSC, 0.1% SDS at 50-65° C. Examples of moderate to low stringency hybridization conditions are well known in the art.

Polynucleotides homologous to the sequences illustrated in the Sequence Listing and figures can be identified, e.g., by hybridization to each other under stringent or under highly stringent conditions. Single stranded polynucleotides hybridize when they associate based on a variety of well characterized physical-chemical forces, such as hydrogen bonding, solvent exclusion, base stacking and the like. The stringency of a hybridization reflects the degree of sequence identity of the nucleic acids involved, such that the higher the stringency, the more similar are the two polynucleotide strands. Stringency is influenced by a variety of factors, including temperature, salt concentration and composition, organic and non-organic additives, solvents, etc. present in both the hybridization and wash solutions and incubations (and number thereof, as described in more detail in the references cited above.

Encompassed by the invention are polynucleotide sequences that are capable of hybridizing to the claimed polynucleotide sequences, including any of the nucleic acid sequences disclosed herein, and fragments thereof under various conditions of stringency (See, for example, Wahl and Berger (1987) Methods Enzymol. 152: 399-407; and Kimmel (1987) Methods Enzymol. 152: 507-511). With regard to hybridization, conditions that are highly stringent, and means for achieving them, are well known in the art. See, for example, Sambrook et al. (1989) “Molecular Cloning: A Laboratory Manual” (2nd ed., Cold Spring Harbor Laboratory); Berger and Kimmel, eds., (1987) “Guide to Molecular Cloning Techniques”, In Methods in Enzymology: 152: 467-469; and Anderson and Young (1985) “Quantitative Filter Hybridisation.” In: Hames and Higgins, ed., Nucleic Acid Hybridisation, A Practical Approach. Oxford, IRL Press, 73-111.

Stability of DNA duplexes is affected by such factors as base composition, length, and degree of base pair mismatch. Hybridization conditions may be adjusted to allow DNAs of different sequence relatedness to hybridize. The melting temperature (Tm) is defined as the temperature when 50% of the duplex molecules have dissociated into their constituent single strands. The melting temperature of a perfectly matched duplex, where the hybridization buffer contains formamide as a denaturing agent, may be estimated by the following equation: DNA-DNA: Tm(° C.)=81.5+16.6(log [Na+])+0.41(% G+C)−0.62(% formamide)−500/L (1) DNA-RNA: Tm(° C.)=79.8+18.5(log [Na+])+0.58(% G+C)+0.12(% G+C).sup.2-0.5(% formamide)-820/L (2) RNA-RNA: Tm(C)=79.8+18.5(log [Na+])+0.58(% G+C)+0.12(% G+C).sup.2-0.35(% formamide)-820/L (3) [0194] where L is the length of the duplex formed, [Na+] is the molar concentration of the sodium ion in the hybridization or washing solution, and % G+C is the percentage of (guanine+cytosine) bases in the hybrid. For imperfectly matched hybrids, approximately 1° C. is required to reduce the melting temperature for each 1% mismatch.

Hybridization experiments are generally conducted in a buffer of pH between 6.8 to 7.4, although the rate of hybridization is nearly independent of pH at ionic strengths likely to be used in the hybridization buffer (Anderson et al. (1985) supra). In addition, one or more of the following may be used to reduce non-specific hybridization: sonicated salmon sperm DNA or another non-complementary DNA, bovine serum albumin, sodium pyrophosphate, sodium dodecylsulfate (SDS), polyvinyl-pyrrolidone, ficoll and Denhardt's solution. Dextran sulfate and polyethylene glycol 6000 act to exclude DNA from solution, thus raising the effective probe DNA concentration and the hybridization signal within a given unit of time. In some instances, conditions of even greater stringency may be desirable or required to reduce non-specific and/or background hybridization. These conditions may be created with the use of higher temperature, lower ionic strength and higher concentration of a denaturing agent such as formamide.

Stringency conditions can be adjusted to screen for moderately similar fragments such as homologous sequences from distantly related organisms, or to highly similar fragments. The stringency can be adjusted either during the hybridization step or in the post-hybridization washes. Salt concentration, formamide concentration, hybridization temperature and probe lengths are variables that can be used to alter stringency (as described by the formula above). As a general guidelines high stringency is typically performed at Tm-5° C. to Tm-20° C., moderate stringency at Tm-20° C. to Tm-35° C. and low stringency at Tm-35° SC to Tm-50° C. for duplex>150 base pairs. Hybridization may be performed at low to moderate stringency (25-50° C. below Tm), followed by post-hybridization washes at increasing stringencies. Maximum rates of hybridization in solution are determined empirically to occur at Tm-25° C. for DNA-DNA duplex and Tm-15° C. for RNA-DNA duplex. Optionally, the degree of dissociation may be assessed after each wash step to determine the need for subsequent, higher stringency wash steps.

High stringency conditions may be used to select for nucleic acid sequences with high degrees of identity to the disclosed sequences. An example of stringent hybridization conditions obtained in a filter-based method such as a Southern or northern blot for hybridization of complementary nucleic acids that have more than 100 complementary residues is about 5° C. to 20° C. lower than the thermal melting point (Tm) for the specific sequence at a defined ionic strength and pH. Conditions used for hybridization may include about 0.02 M to about 0.15 M sodium chloride, about 0.5% to about 5% casein, about 0.02% SDS or about 0.1% N-laurylsarcosine, about 0.02 M to about 0.03 M sodium citrate, at hybridization temperatures between about 50° C. and about 70° C. In certain embodiments, high stringency conditions are about 0.02 M sodium chloride, about 0.5% casein, about 0.02% SDS, about 0.001 M sodium citrate, at a temperature of about 50° C. Nucleic acid molecules that hybridize under stringent conditions will typically hybridize to a probe based on either the entire DNA molecule or selected portions, e.g., to a unique subsequence, of the DNA.

Stringent salt concentration will ordinarily be less than about 750 mM NaCl and 75 mM trisodium citrate. Increasingly stringent conditions may be obtained with less than about 500 mM NaCl and 50 mM trisodium citrate, to even greater stringency with less than about 250 mM NaCl and 25 mM trisodium citrate. Low stringency hybridization can be obtained in the absence of organic solvent, e.g., formamide, whereas in certain embodiments high stringency hybridization may be obtained in the presence of at least about 35% formamide, and in other embodiments in the presence of at least about 50% formamide. In certain embodiments, stringent temperature conditions will ordinarily include temperatures of at least about 30° C., and in other embodiment at least about 37° C., and in other embodiments at least about 42° C. with formamide present. Varying additional parameters, such as hybridization time, the concentration of detergent, e.g., sodium dodecyl sulfate (SDS) and ionic strength, are well known to those skilled in the art. Various levels of stringency are accomplished by combining these various conditions as needed. In a certain embodiment, hybridization will occur at 30° C. in 750 mM NaCl, 75 mM trisodium citrate, and 1% SDS. In another embodiment, hybridization will occur at 37° C. in 500 mM NaCl, 50 mM trisodium citrate, 1% SDS, 35% formamide. In another embodiment, hybridization will occur at 42C in 250 mM NaCl, 25 mM trisodium citrate, 1% SDS, 50% formamide. Useful variations on these conditions will be readily apparent to those skilled in the art.

The washing steps that follow hybridization may also vary in stringency; the post-hybridization wash steps primarily determine hybridization specificity, with the most critical factors being temperature and the ionic strength of the final wash solution. Wash stringency can be increased by decreasing salt concentration or by increasing temperature. Stringent salt concentration for the wash steps can be less than about 30 mM NaCl and 3 mM trisodium citrate, and in certain embodiments less than about 15 mM NaCl and 1.5 mM trisodium citrate. For example, the wash conditions may be under conditions of 0.1×SSC to 2.0×SSC and 0.1% SDS at 50-65° C., with, for example, two steps of 10-30 min. One example of stringent wash conditions includes about 2.0×SSC, 0.1% SDS at 65° C. and washing twice, each wash step being about 30 min. The temperature for the wash solutions will ordinarily be at least about 25° C., and for greater stringency at least about 42° C. Hybridization stringency may be increased further by using the same conditions as in the hybridization steps, with the wash temperature raised about 3° C. to about 5° C., and stringency may be increased even further by using the same conditions except the wash temperature is raised about 6° C. to about 9° C. For identification of less closely related homolog, wash steps may be performed at a lower temperature, e.g., 50° C.

An example of a low stringency wash step employs a solution and conditions of at least 25° C. in 30 mM NaCl, 3 mM trisodium citrate, and 0.1% SDS over 30 min. Greater stringency may be obtained at 42° C. in 15 mM NaCl, with 1.5 mM trisodium citrate, and 0.1% SDS over 30 min. Even higher stringency wash conditions are obtained at 65° C.-68° C. in a solution of 15 mM NaCl, 1.5 mM trisodium citrate, and 0.1% SDS. Wash procedures will generally employ at least two final wash steps. Additional variations on these conditions will be readily apparent to those skilled in the art.

Stringency conditions can be selected such that an oligonucleotide that is perfectly complementary to the coding oligonucleotide hybridizes to the coding oligonucleotide with at least about a 5-10× higher signal to noise ratio than the ratio for hybridization of the perfectly complementary oligonucleotide to a nucleic acid. It may be desirable to select conditions for a particular assay such that a higher signal to noise ratio, that is, about 15× or more, is obtained. Accordingly, a subject nucleic acid will hybridize to a unique coding oligonucleotide with at least a 2× or greater signal to noise ratio as compared to hybridization of the coding oligonucleotide to a nucleic acid encoding known polypeptide. The particular signal will depend on the label used in the relevant assay, e.g., a fluorescent label, a calorimetric label, a radioactive label, or the like. Labeled hybridization or PCR probes for detecting related polynucleotide sequences may be produced by oligolabeling, nick translation, end-labeling, or PCR amplification using a labeled nucleotide.

Screening Assays and Diagnostic methods

Regulators (or modulators) of R-spondin 4 polypeptide molecules, according to the invention, can be compounds that affect the activity of a R-spondin 4 polypeptide molecule or a variant thereof, in vivo and/or in vitro. Regulators can be agonists and antagonists of a R-spondin 4 polypeptide molecule or a variant thereof, and can be compounds that exert their effect on the activity of R-spondin 4 or a R-spondin 4 mutant described above via the expression, via post-translational modifications or by other means.

Agonists of a R-spondin 4 molecule or a variant thereof, are molecules which, when bound to R-spondin 4, increase or prolong the activity of R-spondin 4 or a variant thereof. Agonists of R-spondin 4 or a R-spondin 4 mutant described above include proteins, nucleic acids, carbohydrates, small molecules, or any other molecule which activate R-spondin 4 or a variant thereof.

Antagonists of a R-spondin 4 molecule or a variant thereof, are molecules which, when bound to R-spondin 4, decrease the amount or the duration of the activity of R-spondin 4 or a R-spondin 4 mutant described above. Antagonists include proteins, nucleic acids, carbohydrates, antibodies, small molecules, or any other molecule which decrease the activity of R-spondin 4 or a variant thereof, such as a R-spondin 4 mutant described above.

The term “modulate”, as it appears herein, refers to a change in the activity of a polypeptide of R-spondin 4 or a variant thereof. For example, modulation may cause an increase or a decrease in protein activity, binding characteristics, or any other biological, functional, or immunological properties of R-spondin 4 or a variant thereof.

R-spondin 4 is a protein involved in Wnt and Frizzled signaling. Monitoring Wnt signaling in a cell may be carried out in vivo or in vitro according to methods known in the art. It can be effectively achieved in a Wnt-responsive cell in tissue culture. Since Wnt signaling is conserved in vertebrates and invertebrates, methods for activating the same may be carried out in tissue culture cells derived from either vertebrates or non-vertebrates. Examples of vertebrate cell lineages in which Wnt signaling is conserved include human, mouse, Xenopus, chicken and zebrafish. Examples of invertebrate cell lineages in which Wnt signaling is conserved include C. elegans and Drosophila. Wnt signaling can lead to the activation of the canonical, Wnt/β-catenin, pathway. The activation of Wnt signaling can be measured either by the increase in the cytoplasmic accumulation of β-catenin or the activation of T-cell factor/lymphoid enhancer factor (TCF/LEF)-reporter genes (Wodarz et al. (1998) Annu. Rev. Cell Dev. Biol. 14:59-88; Miller (2002) Genome Biol. 3: reviews 3001.1-3001.15). Microinjection of mRNA into Xenopus embryos is generally used to validate Wnt signaling; and in such a system, Wnt1-induced axis duplication is inhibited by SFRPs (Lin et al. (1997) Proc. Natl. Acad. Sci. USA 94:11196-11200).

Biochemical studies utilizing the co-immunoprecipitation or ELISA methods are also used to identify the interaction of Wnt and with other Wnt-pathway associated proteins (Lin et al. (1997) Proc. Natl. Acad. Sci. USA 94:11196-11200). Additional assays for monitoring Wnt signaling activity include, but are not limited to modulation of another Wnt-responsive transcription factor, LEF, as visualized by a reporter gene activity. One example includes the activation of the LEF1 promoter region fused to the luciferase reporter gene (Hsu et al., Mol. Cell. Biol. 18: 4807-18 (1999)); alterations in cell proliferation, cell cycle or apoptosis. There are numerous examples describing Wnt-mediated cellular transformations including Shimizu et al., Cell. Growth Differ. 8: 1349-58 (1997); and stabilization and cellular localization of de-phosphorylated β-catenin as an indicator of Wnt activation (Shimizu et al., 1997). Additional methods can also be found in U.S. Patent Application Publication Nos. 20070072239 and 20070059829, which are hereby Incorporated by reference.

Test compounds or agents which bind to a R-spondin 4 molecule or a variant thereof, and/or have a stimulatory or inhibitory effect on the activity or the expression of a R-spondin 4 molecule or a variant thereof, can be identified by assays that make use of isolated R-spondin 4 molecules or mutants thereof (also referred to as cell-free assays). The various assays can employ a variety of variants of R-spondin 4 molecules (e.g., a biologically active fragment of R-spondin 4, full-length R-spondin 4, a fusion protein which includes all or a portion of R-spondin 4, or a R-spondin 4 mutant previously presented—having the biochemical variations just described, i.e., a fusion protein or fragments thereof). A R-spondin 4 molecule or a variant thereof, such as a R-spondin 4 mutant described above, can be derived from any suitable mammalian species (e.g., a R-spondin 4 protein molecule from human, canine, feline, equine, porcine, bovine, murine, and the like). The assay can be a binding assay comprising direct or indirect measurement of the binding of a test compound or a known R-spondin 4 interacting protein. The assay can also be an activity assay comprising direct or indirect measurement of the activity of a R-spondin 4 molecule or a variant thereof (i.e., a polypeptide or a nucleic acid). The assay can also be an expression assay comprising direct or indirect measurement of the expression of mRNA of R-spondin 4 or a variant thereof, or a R-spondin 4 protein. The various screening assays can be combined with an in vivo assay comprising measuring the effect of the test compound on the symptoms of a nail, hoof, or claw keratin-related abnormality in a subject (for example, anonychia congenita, hyponychia congenita, Cooks syndrome, nail patella syndrome, ectodermal dysplasias, and epidermolysis bullosa).

Specific binding (or specifically binding) can be an interaction between a protein or peptide and an agonist, an antibody, or an antagonist. The interaction is dependent upon the presence of a particular structure of the protein recognized by the binding molecule (i.e., the antigenic determinant or epitope). For example, if an antibody is specific for epitope “A” the presence of a polypeptide containing the epitope A, or the presence of free unlabeled A, in a reaction containing free labeled A and the antibody will reduce the amount of labeled A that binds to the antibody.

The diagnostic assay of the screening methods of the invention can also involve monitoring the expression of a R-spondin 4 molecule or a variant thereof. For example, regulators of the expression of a R-spondin 4 molecule, or a variant thereof, can be identified via contacting a cell with a test compound and determining the expression of R-spondin 4 or R-spondin 4 mutant protein or R-spondin 4 or R-spondin 4 mutant mRNA in the cell. The level of expression of R-spondin 4 or R-spondin 4 mutant protein or R-spondin 4 or R-spondin 4 mutant mRNA in the presence of the test compound is compared to the level of expression of R-spondin 4 or R-spondin 4 mutant protein or R-spondin 4 or R-spondin 4 mutant mRNA in the absence of the test compound. The test compound can then be identified as a regulator of expression of R-spondin 4 or a variant thereof, based on this comparison. For example, when expression of R-spondin 4 or R-spondin 4 mutant protein or R-spondin 4 or R-spondin 4 mutant mRNA is statistically or significantly greater in the presence of the test compound than in its absence, the test compound is identified as a stimulator of expression of R-spondin 4 or R-spondin 4 mutant protein, or R-spondin 4 or R-spondin 4 mutant mRNA (i.e., the R-spondin 4 modulating compound is an agonist).

Alternatively, when expression of R-spondin 4 or R-spondin 4 mutant protein or R-spondin 4 or R-spondin 4 mutant mRNA is statistically or significantly less in the presence of the test compound than in its absence, the compound is identified as an inhibitor of the expression of R-spondin 4 or R-spondin 4 mutant protein or R-spondin 4 or R-spondin 4 mutant mRNA. The level of R-spondin 4 or R-spondin 4 mutant protein or R-spondin 4 or R-spondin 4 mutant mRNA expression in the cells can be determined by methods previously described (i.e., the R-spondin 4 modulating compound is an antagonist).

For example, the invention provides a method for diagnosing anonychia congenita in a subject. Here, the method can comprise testing the subject for a mutation in the R-spondin 4 gene, wherein a DNA sample is obtained from the subject. In one embodiment, the subject is a human. In another embodiment, the mutation can comprise a nucleic acid sequence comprising SEQ ID NO: 11, wherein the first 26 nucleic acid residues (nucleotide at position −9 to nucleotide at position +17) are deleted from SEQ ID NO: 2; a nucleic acid sequence comprising SEQ ID NO: 12, wherein 16 nucleic acid residues (nucleotide at position +95 to nucleotide at position +110) are deleted from SEQ ID NO: 2; a nucleic acid sequence comprising SEQ ID NO: 7, wherein an A>G mutation occurs at nucleic acid position +194 of SEQ ID NO: 2; a nucleic acid sequence comprising SEQ ID NO: 8, wherein a G>T mutation occurs at nucleic acid position +284 of SEQ ID NO: 2; a nucleic acid sequence comprising SEQ ID NO: 9, wherein a T>C mutation occurs at nucleic acid position +319 of SEQ ID NO: 2; a nucleic acid sequence comprising SEQ ID NO: 10, wherein a G>A mutation occurs at nucleic acid position +353 of SEQ ID NO: 2; a nucleic acid sequence comprising SEQ ID NO: 15, wherein an G>A mutation occurs at nucleic acid position +3 of SEQ ID NO: 2; or a combination thereof.

In a further embodiment, the mutation can comprise a nucleic acid encoding a polypeptide molecule comprising an amino acid sequence comprising SEQ ID NO: 3, wherein a Q>R mutation occurs at amino acid position 65 of SEQ ID NO: 1; a nucleic acid encoding a polypeptide molecule comprising an amino acid sequence comprising SEQ ID NO: 4, wherein a C>F mutation occurs at amino acid position 95 of SEQ ID NO: 1; a nucleic acid encoding a polypeptide molecule comprising an amino acid sequence comprising SEQ ID NO: 5, wherein a C>R mutation occurs at amino acid position 107 of SEQ ID NO: 1; a nucleic acid encoding a polypeptide molecule comprising an amino acid sequence comprising SEQ ID NO: 6, wherein a C>Y mutation occurs at amino acid position 118 of SEQ ID NO: 1; a nucleic acid encoding a polypeptide molecule comprising an amino acid sequence comprising SEQ ID NO: 14, wherein a M>I mutation occurs at amino acid position 1 of SEQ ID NO: 1; or a combination thereof.

In particular embodiments, the human R-spondin 4 mutation can comprise a nucleic acid comprising SEQ ID NO: 16, wherein a G>A mutation occurs at nucleic acid position 3077 of SEQ ID NO: 19; a nucleic acid comprising SEQ ID NO: 17, wherein a G>A mutation occurs at nucleic acid position 3711 of SEQ ID NO: 19; a nucleic acid comprising SEQ ID NO: 20, wherein a G>A mutation occurs at nucleic acid position 809 of SEQ ID NO: 19; a nucleic acid comprising SEQ ID NO: 21, wherein a G>A mutation occurs at nucleic acid position 2887 of SEQ ID NO: 19; or a combination thereof. These mutations can give rise to a RSPO4 splice variant. In some embodiments, the splice variant mutants of RSPO4 can arise from a G>A nucleic acid mutation at about nucleotide position 3853 of SEQ ID NO: 19, which lies at the intron 3-exon 3 boundary (see FIG. 9D); from a G>A nucleic acid mutation at about nucleotide position 4797 of SEQ ID NO: 19, which lies at the intron 3-exon 4 boundary (see FIG. 9D); from a G>A nucleic acid mutation at about nucleotide position 4984 of SEQ ID NO: 19, which lies at the intron 4-exon 4 boundary (see FIG. 9D); from a G>A nucleic acid mutation at about nucleotide position 6095 of SEQ ID NO: 19, which lies at the intron 4-exon 5 boundary (see FIG. 9D); or a combination thereof, thus generating a splice site mutant predicted to result in aberrant splicing of RSPO4. The intron-exon boundaries are denoted as red nucleotides that precede or follow the shaded exon nucleic acid sequences (shadowed) in SEQ ID NO: 19

In other embodiments, a mutation (such as a deletion, insertion, or substitution mutation) can occur in a nucleic acid encoding a polypeptide molecule comprising SEQ ID NO: 22 (exon 1 or RSPO4), SEQ ID NO: 23 (exon 2 or RSPO4), SEQ ID NO: 24 (exon 3 or RSPO4), SEQ ID NO: 25 (exon 4 or RSPO4), SEQ ID NO: 26 (exon 5 or RSPO4), or a combination thereof. In a further embodiment, the mutation can attenuate the function of the R-spondin 4 protein or produces a truncated R-spondin protein.

For binding assays, the test compound can be a small molecule which binds to and occupies the active site of a R-spondin 4 polypeptide molecule, or a variant thereof, such as a R-spondin 4 mutant described above. This can make the ligand binding site inaccessible to substrate such that normal biological activity is prevented. Examples of such small molecules include, but are not limited to, small peptides, fragments (such as those corresponding to R-spondin 4 exon 1, exon 2, exon 3, exon 4, or exon 5 that comprise SEQ ID NO: 22, 23, 24, 25, or 26, respectively) or peptide-like molecules. Potential ligands which bind to a polypeptide of the invention include, but are not limited to, the natural ligands of known R-spondin 4 homologues, paralogues, or orthologues. In binding assays, either the test compound or the R-spondin 4 polypeptide molecule or a variant thereof can comprise a detectable label, such as a fluorescent, radioisotopic, chemiluminescent, or enzymatic label (for example, alkaline phosphatase, horseradish peroxidase, or luciferase). Detection of a test compound which is bound to a polypeptide of R-spondin 4 or a R-spondin 4 mutant described above can then be determined via direct counting of radioemmission, by scintillation counting, or by determining conversion of an appropriate substrate to a detectable product.

Determining the ability of a test compound to bind to a R-spondin 4 molecule or a variant thereof, such as a R-spondin 4 mutant described above, also can be accomplished using real-time Bimolecular Interaction Analysis (BIA) [McConnell, (1992); Sjolander, (1991)]. BIA is a technology for studying biospecific interactions in real time, without labeling any of the interactants (for example, BIA-core™). Changes in the optical phenomenon surface plasmon resonance (SPR) can be used as an indication of real-time reactions between biological molecules.

Test compounds can be tested for the ability to increase or decrease the activity of a R-spondin 4 polypeptide molecule, or a variant thereof. Activity of a R-spondin 4 molecule or a R-spondin 4 mutant molecule described above can be measured after contacting either a purified R-spondin 4 molecule or a variant thereof, a cell membrane preparation, or an intact cell with a test compound. A test compound that decreases the activity of a R-spondin 4 molecule or a variant thereof, such as a R-spondin 4 mutant described above, by at least about 10%, 20%, 30%, 40%, 50%, 60%, 70%, 75%, 80%, 90%, 95% or 100% is identified as a potential agent for decreasing the activity of a R-spondin 4 molecule or a variant thereof. A test compound that increases the activity of a R-spondin 4 molecule or a variant thereof, such as a R-spondin 4 mutant described above, by at least about 10%, 20%, 30%, 40%, 50%, 60%, 70%, 75%, 80%, 90%, 95% or 100% is identified as a potential agent for increasing the activity of R-spondin 4 or a variant thereof.

Pharmaceutical Compositions and Administration for Therapy

This invention further pertains to agents identified by the above-described screening assays and uses thereof for treatments as described herein. The R-spondin 4 polynucleotide or polypeptide molecules of the invention can be formulated into composition suitable for delivery.

The nucleic acid molecules, polypeptides, small molecules, compounds, antibodies, and the like, of the invention can be incorporated into pharmaceutical compositions suitable for administration. Such compositions typically comprise the nucleic acid molecule, protein, small molecule, compound, or antibody and a pharmaceutically acceptable carrier.

According to the invention, a pharmaceutically acceptable carrier can comprise any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention can also comprise pharmaceutical compositions comprising a regulator (or modulator) of R-spondin 4 expression or activity (and/or a regulator of the activity or expression of a protein in the R-spondin 4-mediated signaling pathway) as well as methods for preparing such compositions by combining one or more such regulators and a pharmaceutically acceptable carrier. The invention also provides for a kit that comprises a R-spondin 4 modulator identified using the screening assays described above, packaged with instructions for use. For modulators that are antagonists of the activity of R-spondin 4 or a variant thereof, such as a R-spondin 4 mutant described above, or which reduce the expression of R-spondin 4 or a R-spondin 4 mutant previously described, the instructions would specify use of the pharmaceutical composition for decreasing growth of a claw or hoof, such as in a dog, cat, bird, horse, cow, pig, and the like.

For regulators that are agonists of the activity of a R-spondin 4 molecule or a variant thereof, or increase the expression of a R-spondin 4 polypeptide molecule or a R-spondin 4 mutant previously described, the instructions would specify use of the pharmaceutical composition for regulating the growth of keratinized structures (such as a nail, a hoof, or a claw). In one embodiment, the instructions would specify use of the composition for the treatment of nail, hoof, or claw keratin-related abnormalities in a subject. In another embodiment, the instructions would specify use of the pharmaceutical composition for promoting the growth of keratinized structures in a subject. In some embodiments, the instructions would specify use of the pharmaceutical composition for strengthening keratinized structures in a subject. In a further embodiment, the instructions would specify use of the pharmaceutical composition for the weakening of keratinized structures. For example, administering a R-spondin 4 agonist could increase the rate of nail growth in a subject afflicted with a keratin related abnormality (i.e., a nail hypoplastic abnormality).

An antagonist or agonist of a R-spondin 4 molecule or a variant thereof, may be produced using methods which are generally known in the art. In a particular embodiment, a purified R-spondin 4 polypeptide molecule, or a variant thereof, may be used to produce antibodies or to screen libraries of pharmaceutical agents to identify those which specifically bind R-spondin 4 molecules. Antibodies to R-spondin 4 may also be generated using methods that are well known in the art. Non-limiting examples of such antibodies may include polyclonal, monoclonal, chimeric, single chain antibodies, Fab fragments, and fragments produced by a Fab expression library. Neutralizing antibodies, such as those that inhibit dimer formation can be of particular therapeutic use.

In one embodiment, a polynucleotide encoding a R-spondin 4 molecule, or any fragment or complement thereof, may be used for therapeutic purposes. For example, the complement of the polynucleotide encoding a R-spondin 4 molecule, or a variant thereof, may be used in situations in which it would be desirable to block the transcription of the mRNA. It may be particularly useful that cells be transformed with sequences complementary to polynucleotides encoding R-spondin 4 or a variant thereof. Thus, complementary molecules or fragments may be used to modulate the activity of R-spondin 4 or a variant thereof, or to achieve regulation of gene function. Such technology is well known in the art (see discussion above), and sense or antisense oligonucleotides or larger fragments can be designed from various locations along the coding or control regions of sequences encoding R-spondin 4 or a variant thereof, such as a R-spondin 4 mutant described above. For example, fragments can be designed from various locations along the coding or control regions of sequences encoding either exon 1, exon 2, exon 3, exon 4, or exon 5 of R-spondin 4. For example, the antisense RNA or siRNA molecule can be directed to a particular portion of R-spondin 4 (such as nucleic acid sequences of exon 1, exon 2, exon 3, exon 4, or exon 5 of the RSPO4 gene (SEQ ID NOS: 27, 28, 29, 30, or 31, respectively)).

Any of the therapeutic methods described above may be applied to any subject in need of such therapy, including, for example, a mammal such as a dog, a cat, a cow, a horse, a rabbit, a monkey, a pig, a sheep, a goat, and most particularly, a human.

A pharmaceutical composition containing a R-spondin 4 molecule or a variant thereof, can be administered in conjunction with a pharmaceutically acceptable carrier, for any of the therapeutic effects discussed above. Such pharmaceutical compositions may comprise a R-spondin 4 molecule or a variant thereof, antibodies to R-spondin 4 or a variant thereof, and fragments, peptidomimetics, agonists, antagonists, or inhibitors of R-spondin 4 molecules.

The compositions may be administered alone or in combination with at least one other agent, such as a stabilizing compound, which may be administered in any sterile, biocompatible pharmaceutical carrier including, but not limited to, saline, buffered saline, dextrose, and water. The compositions may be administered to a patient alone, or in combination with other agents, drugs or hormones.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, particularly useful preparation methods are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art

In some embodiments, the composition of the invention may be applied via transdermal delivery systems, which slowly releases the active compound for percutaneous absorption. Permeation enhancers may be used to facilitate transdermal penetration of the active factors in the conditioned media. Transdermal patches are described in for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

In other embodiments, the compositions of the present invention can be useful for regulating keratinous tissue, in particularly keratinous tissue afflicted with a keratin related abnormality, particularly nail, hoof, or claw conditions. Such regulation of keratinous tissue conditions (for example a keratin-related abnormality discussed above) can include prophylactic and therapeutic regulation. For example, regulating keratinous issue can include, but is not limited to thickening keratinous tissue (i.e., building the keratinous layers of the nail, hoof, or claw) or weakening keratinous tissue, such as a hoof or claw.

Regulating a keratin related abnormality (for example those previously described) can be practiced by applying a composition in the form of a lotion, cream, gel, foam, ointment, paste, serum, stick, emulsion, spray, conditioner, tonic, cosmetic, nail polish, or the like to portions of the tissue. The compositions are preferably intended to be left on the keratin structure for some esthetic, prophylactic, therapeutic or other benefit (i.e., a “leave-on” composition). After applying the composition to the nail, hoof, or claw, it is can be left on the tissue for a period of at least about 15 minutes, or at least about 30 minutes, or at least about 1 hour, and more particularly for at least several hours (for example, up to about 12 hours). The composition can be applied with the fingers or with an implement or device (e.g., pad, cotton ball, applicator pen, spray applicator, and the like).

For example, R-spondin 4 modulator molecules of the present invention may be used in nail polish compositions for treating fingernails and toenails, a hoof, or claws having a keratin-related abnormality. An effective amount of a R-spondin 4 modulator molecule for use in a nail polish composition can be a proportion of from about 0.001% to about 20% by weight relative to the total weight of the composition. Components of a cosmetically acceptable medium for nail polishes are described by Philippe et al. in U.S. Pat. No. 6,280,747. The nail polish composition typically contains a solvent and a film forming substance, such as cellulose derivatives, polyvinyl derivatives, acrylic polymers or copolymers, vinyl copolymers and polyester polymers. Additionally, the nail polish may contain a plasticizer, such as tricresyl phosphate, benzyl benzoate, tributyl phosphate, butyl acetyl ricinoleate, triethyl citrate, tributyl acetyl citrate, dibutyl phthalate or camphor.

A therapeutically effective amount can be the amount of a R-spondin 4 modulator molecule (i.e., a R-spondin 4 binding protein, small molecule, compound, a R-spondin 4 variant, fragment, or peptidomimetic thereof) which is capable of producing a medically desirable result in a treated subject. As is well known in the medical arts, the dosage for any one patient depends upon many factors, including the patient's size, body surface area, age, the particular compound to be administered, sex, time and route of administration, general health, and other drugs being administered concurrently. Dosages will vary, but a preferred dosage for administration of a R-spondin 4 modulator molecule, for example a R-spondin 4 polynucleotide of the invention, can be from approximately 10⁶ to 10¹² copies of the polynucleotide molecule. This dose can be repeatedly administered, as needed.

It should be understood that the embodiments of the present invention shown and described in the specification are only preferred embodiments of the inventor who is skilled in the art and are not limiting in any way. Therefore, various changes, modifications or alterations to these embodiments may be made or resorted to without departing from the spirit of the invention and the scope of the following claims.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods may be utilized to obtain similar results.

### Example 1

### R-spondin 4 (RSPO4) a Secreted Protein Implicated in Wnt Signaling, is Mutated in Inherited Anonychia

Anonychia/hyponychia congenita (OMIM 206800) is a rare autosomal recessive condition in which the only presenting phenotype is the absence or severe hypoplasia of all fingernails and toenails. Genome-wide mapping using Affymetrix 10K SNP arrays, revealed a region of linkage on chromosome 20p13 with a maximum LOD score of >4.0 in one Pakistani, one Finnish and one Irish family. Further recombination mapping in unrelated Pakistani families reduced the minimal region harbouring the disease gene to a small ˜300 kb region including only four genes. Homozygous or compound heterozygous mutations were identified in eight anonychia pedigrees in the gene encoding RSPO4, a secreted protein implicated in wnt signalling. RSPO4 expression was specifically localized to developing e14.5 mouse nail mesenchyme, suggesting a crucial role in nail morphogenesis.

Methods

Clinical details: Informed consent was obtained from all subjects and approval for this study was provided by the East London and City Health Authority (ELCHA) and through Columbia University.

Linkage analysis: Genome-wide linkage analysis was performed using 400 microsatellite markers that were analysed on the ABI3700. Fine mapping was performed using the Affymetrix Human Mapping 10Kv2 SNP array, DNA samples were processed in accordance with the standard GeneChip Mapping 10K Xba Assay protocol. Briefly, 350 ng of DNA was digested with XbaI and ligation to the XbaI adaptor prior to PCR amplification by use of AmpliTaq Gold with Buffer II (Applied Biosystems). For each DNA sample, four 100-ml PCRs were set up to obtain sufficient purified PCR product (20 mg), by use of Ultrafree MC filtration column (Millipore), for subsequent fragmentation with DNase I. Fragmentation was visualized by 4% agarose-gel electrophoresis to confirm the production of 50-100-bp PCR fragments prior to 3′ labeling with biotin and hybridization to the SNP array. Hybridized arrays were processed with an Affymetrix Fluidics Station 450, and fluorescence signals were detected using the Affymetrix GeneChip Scanner 3000. Raw SNP call data were exported to Microsoft Excel for analysis. Data management and cleaning was done with the ALOHOMORA package (Ruschendorf, F. & Nurnberg, P. ALOHOMORA: a tool for linkage analysis using 10K SNP array data. Bioinformatics 21, 2123-5 (2005)), GRR (Abecasis, G. R., Chemy, S. S., Cookson, W. O. & Cardon, L. R. GRR: graphical representation of relationship errors. Bioinformatics 17, 742-3 (2001)), and PedCheck (O'Connell, J. R. & Weeks, D. E. PedCheck: a program for identification of genotype incompatibilities in linkage analysis. Am J Hum Genet. 63, 259-66 (1998)). Parametric multipoint linkage analysis was performed with Allegro (Gudbjartsson, D. F., Jonasson, K., Frigge, M. L. & Kong, A. Allegro, a new computer program for multipoint linkage analysis. Nat Genet. 25, 12-3 (2000)) using a recessive model and complete penetrance.

Mutation analysis: The five coding exons of RSPO4 were amplified from affected individuals. PCR primers were designed using the Ensembl database and PRIMER3 software (http://frodo.wi.mit.edu/cgi-bin/primer3/primer3_www.cgi). PCR products were cleaned using ExoSAP-IT (Amersham Pharmacia Biotech), followed by sequencing with either the forward or reverse primer using BigDye BigDye Terminator v3.1 Cycle Sequencing Kit and analysed on the ABI PRISM® 3700 DNA Analyzer (Applied Biosystems). Sequence analysis was performed using Phred, Phrap, and Consed, and variants were detected using reference sequences taken from the Ensembl Genome Browser (Ewing B, Green P. Base-calling of automated sequencer traces using Phred. II. Error probabilities. Genome Res 8:186-194 (1998); Ewing B, Hillier L, Wendl M C, Green P. Base-calling of automated sequencer traces using Phred. I. Accuracy assessment. Genome Res 8:175-185 (1998); Gordon D, Abajian C, Green P. Consed: a graphical tool for sequence finishing. Genome Res 8:195-202 (1998)).

In situ hybridization: Approval for the mouse work was obtained through Columbia University.

GenBank Accession numbers: RSPO4 mRNA NM_{—}001029871, protein NP_{—}001025042

Results and Discussion

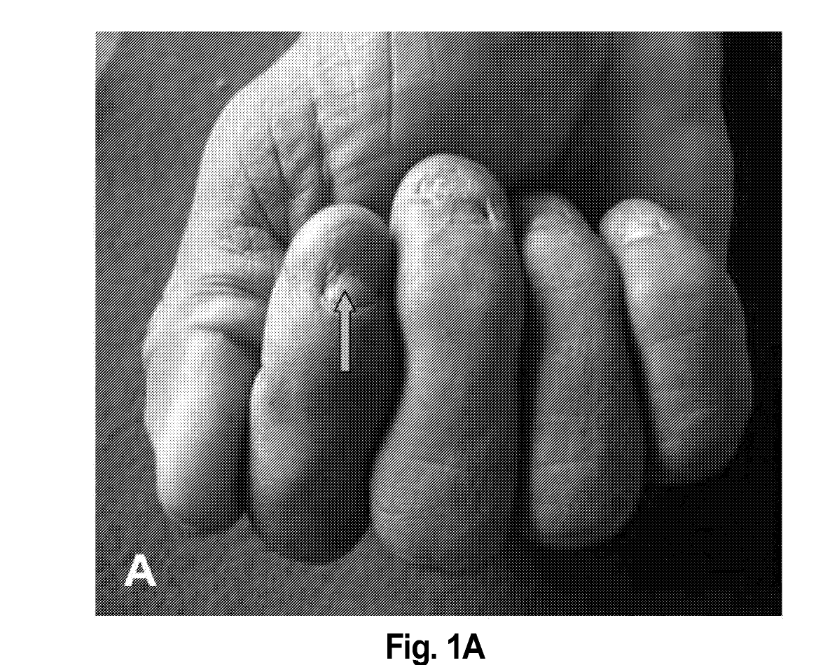

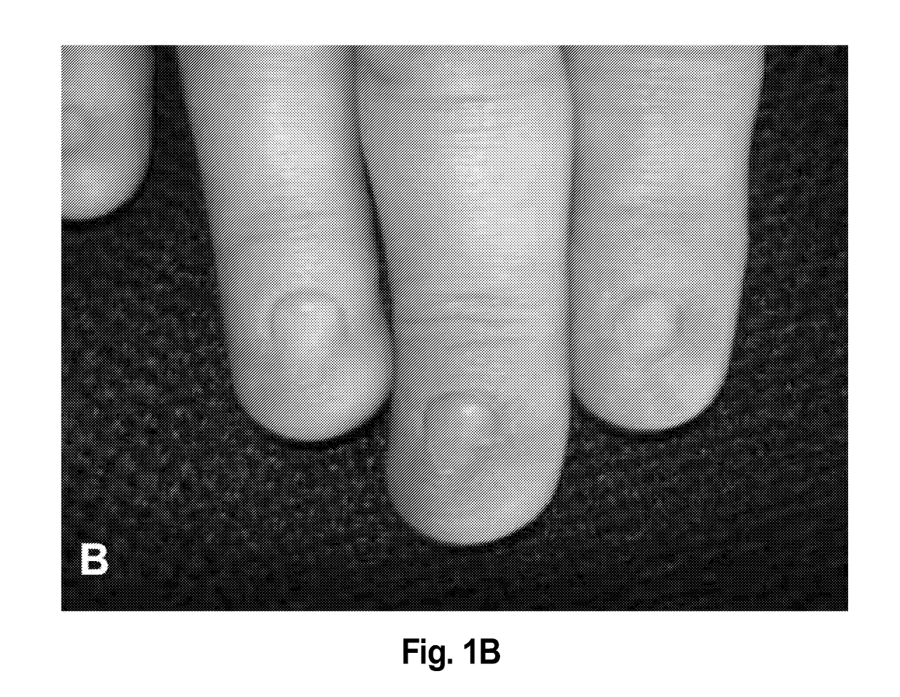

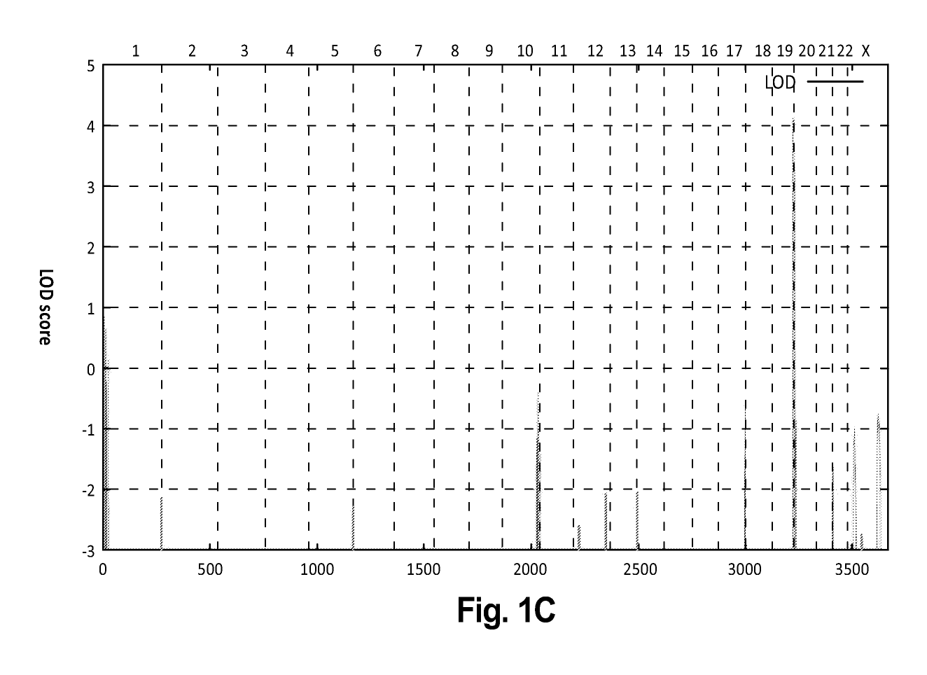

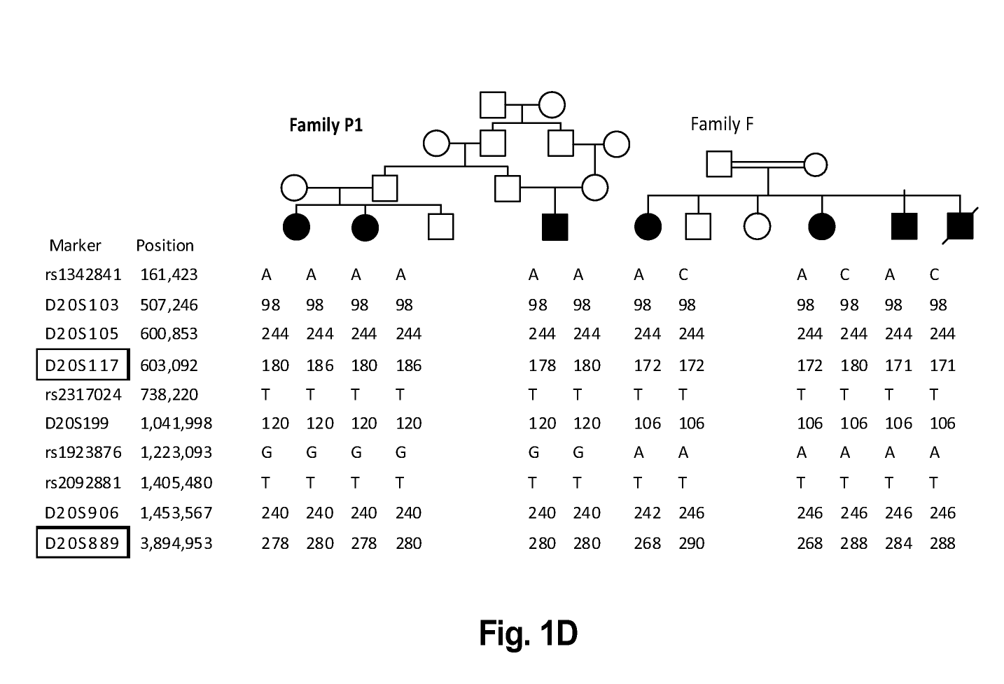

We studied a number of families that show recessive inheritance of a combination of isolated anonychia/hyponychia. A consanguineous family of Pakistani origin (PI) presented with two affected siblings and an affected first cousin. A consanguineous Finnish family (F), reported previously (Hopsu-Havu, V. K. & Jansen, C. T. Anonychia congenita. Arch Dermatol 107, 752-3 (1973)), had four out of ten siblings affected. An additional family of Irish descent (I) with three affected siblings showed no evidence of consanguinity. All patients exhibited complete absence of the nail plate, with only the nail bed and a swollen nail matrix present (FIG. 1A and FIG. 1B).

An initial genome-wide linkage analysis of only the affected individuals from the Pakistani (P1), Finnish and Irish families, performed using 400 microsatellite markers, did not reveal any regions of linkage. Therefore, higher resolution mapping was applied on the same affected individuals, as well as on two unaffecteds, in a whole-genome sampling analysis (WGSA) (Kennedy, G. C. et al. Large-scale genotyping of complex DNA. Nat Biotechnol 21, 1233-7 (2003)) approach using the Affymetrix Human Mapping 10Kv2 SNP array. Data management and cleaning was done with the ALOHOMORA package (Ruschendorf, F. & Nurnberg, P. ALOHOMORA: a tool for linkage analysis using 10K SNP array data. Bioinformatics 21, 2123-5 (2005)), GRR (Abecasis, G. R., Chemy, S. S., Cookson, W. O. & Cardon, L. R. GRR: graphical representation of relationship errors. Bioinformatics 17, 742-3 (2001)), and PedCheck (O'Connell, J. R. & Weeks, D. E. PedCheck: a program for identification of genotype incompatibilities in linkage analysis. Am J Hum Genet. 63, 259-66 (1998)).

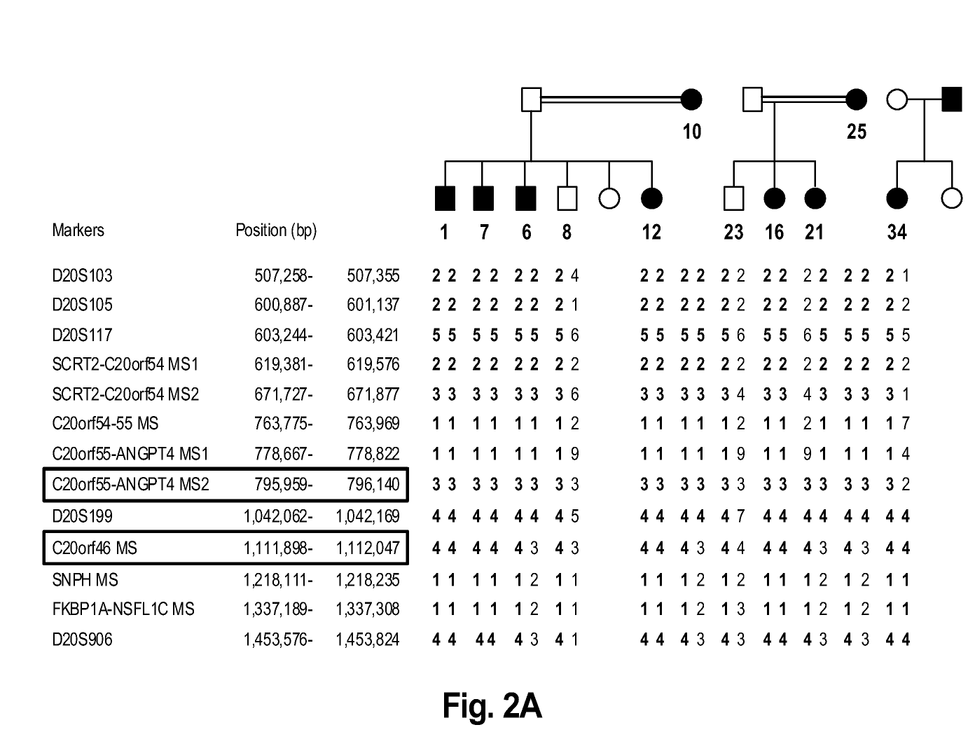

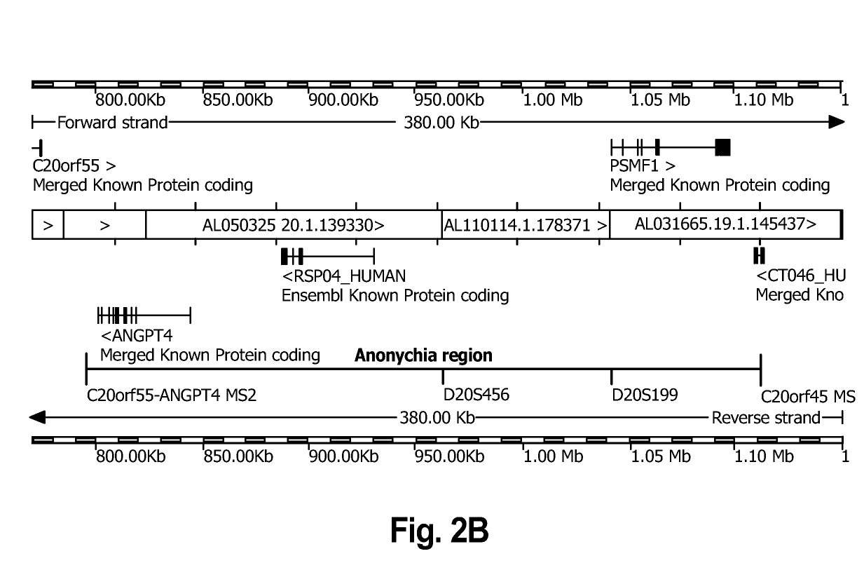

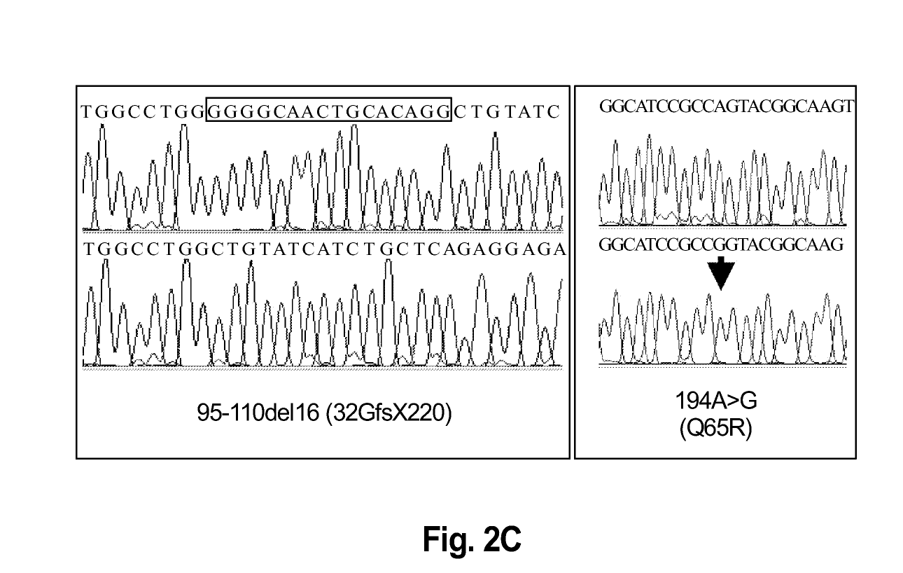

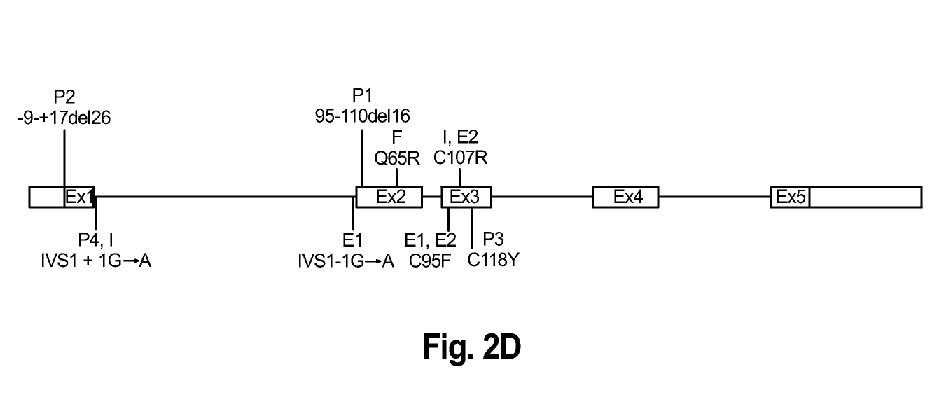

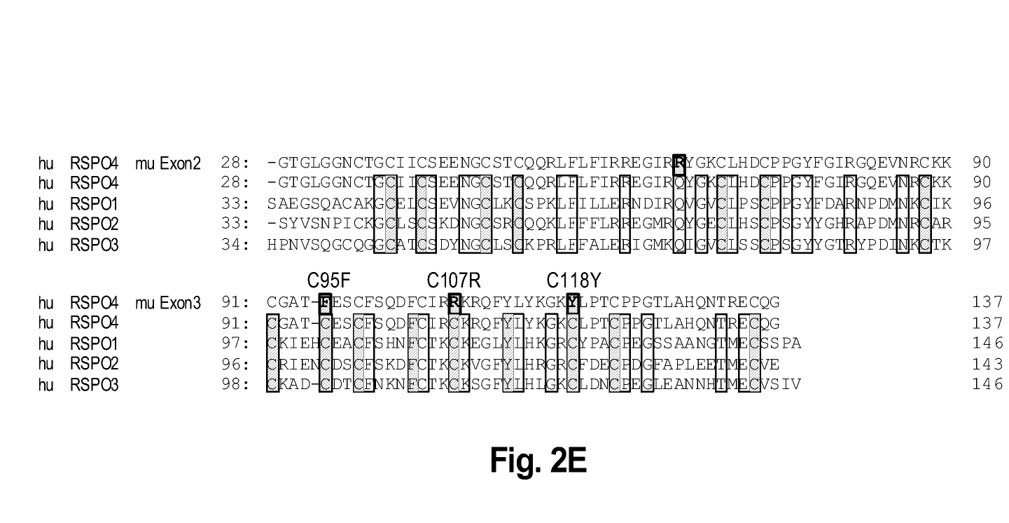

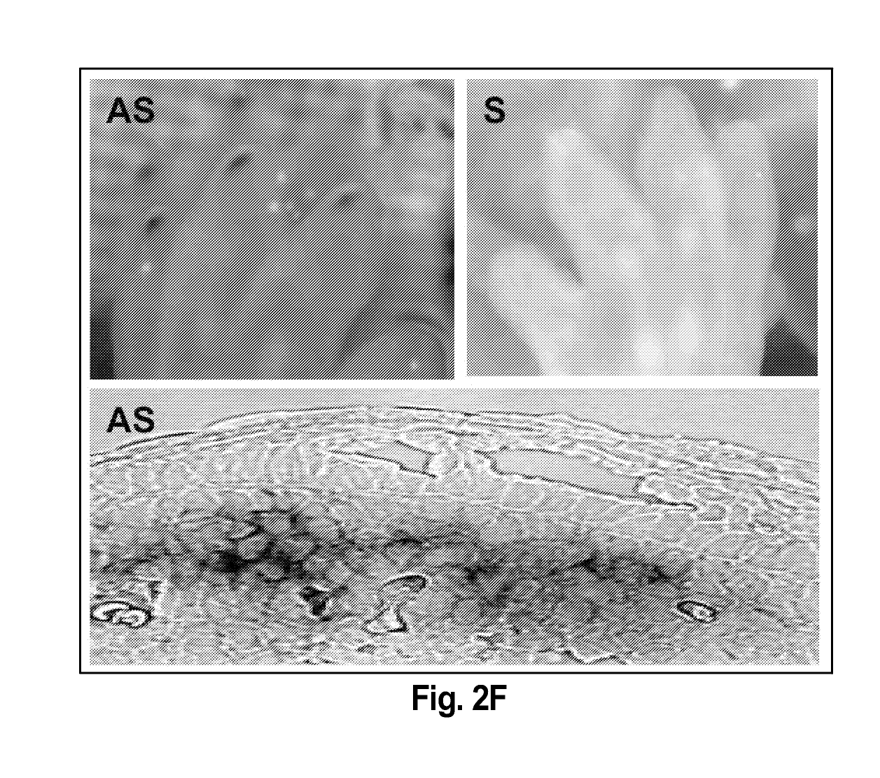

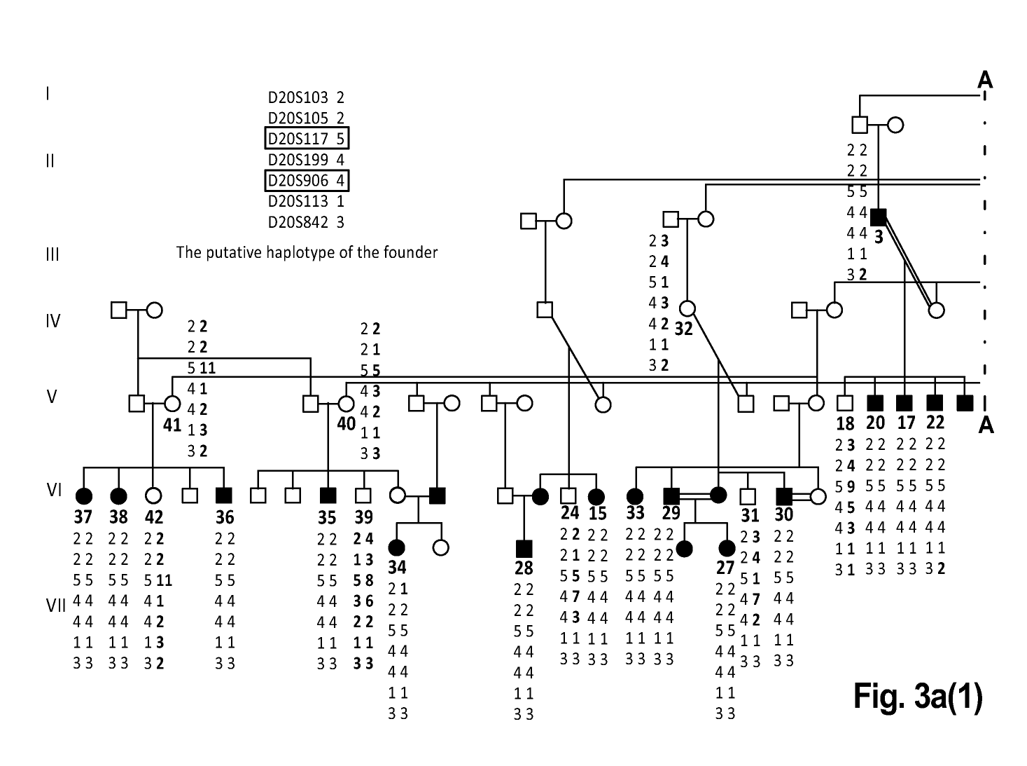

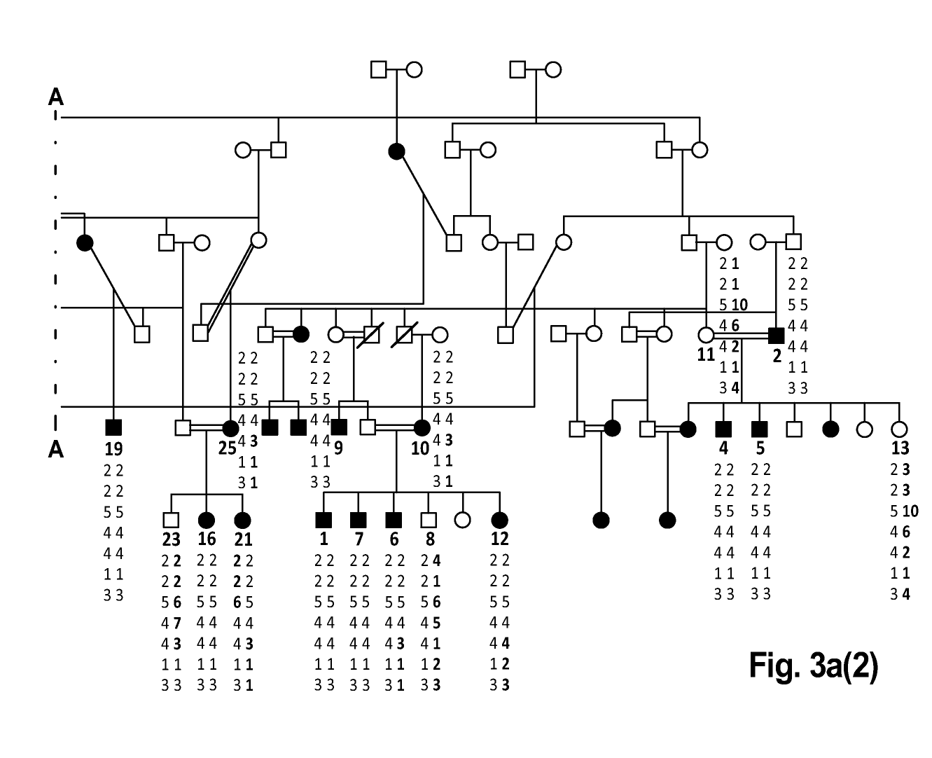

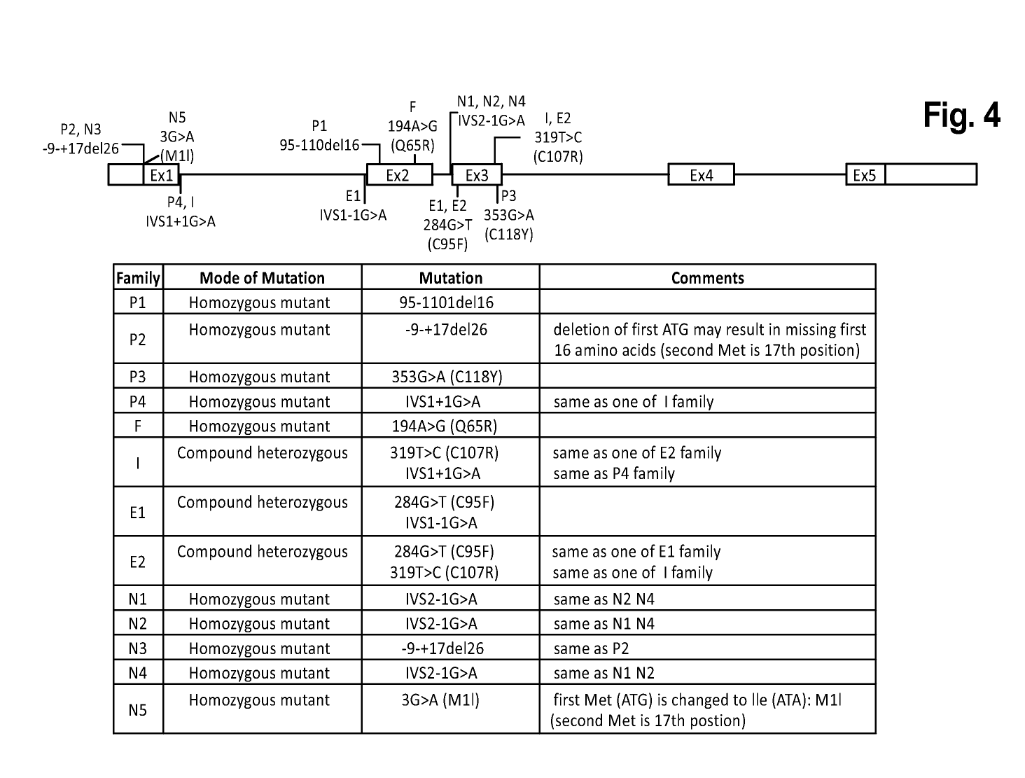

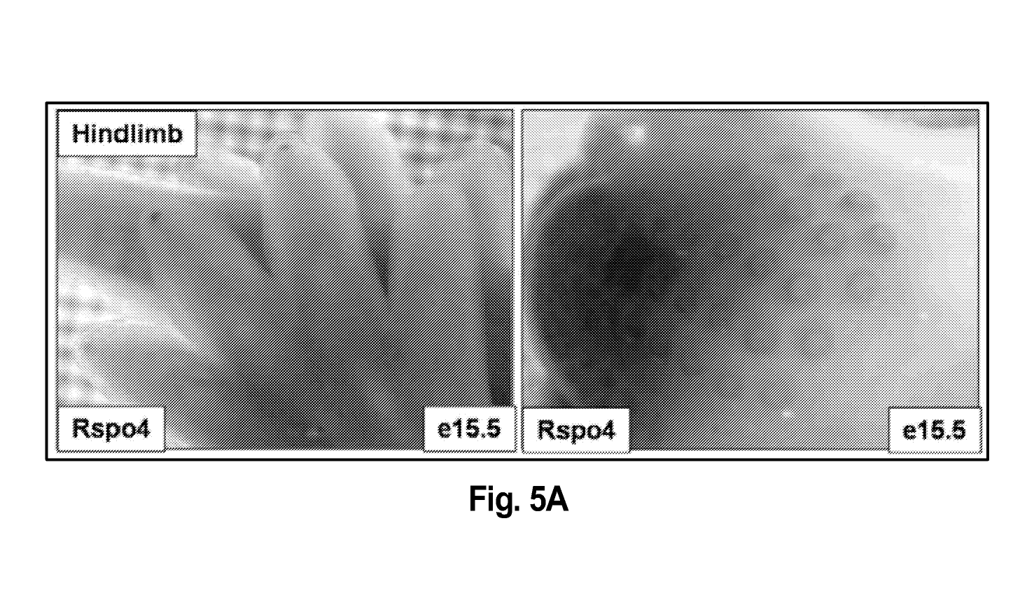

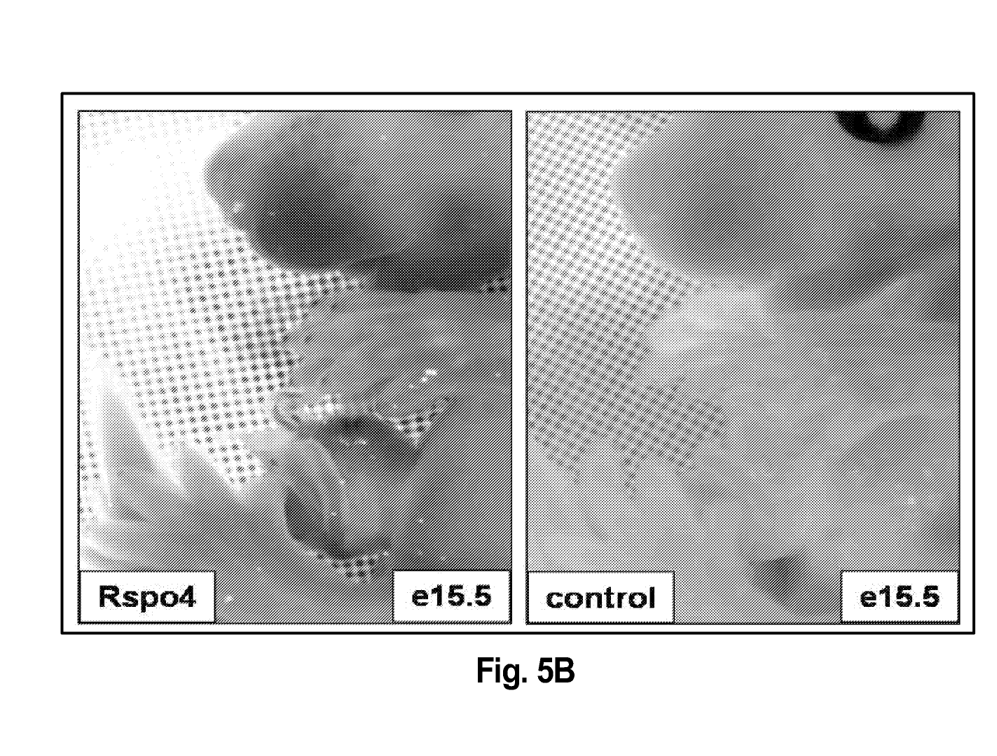

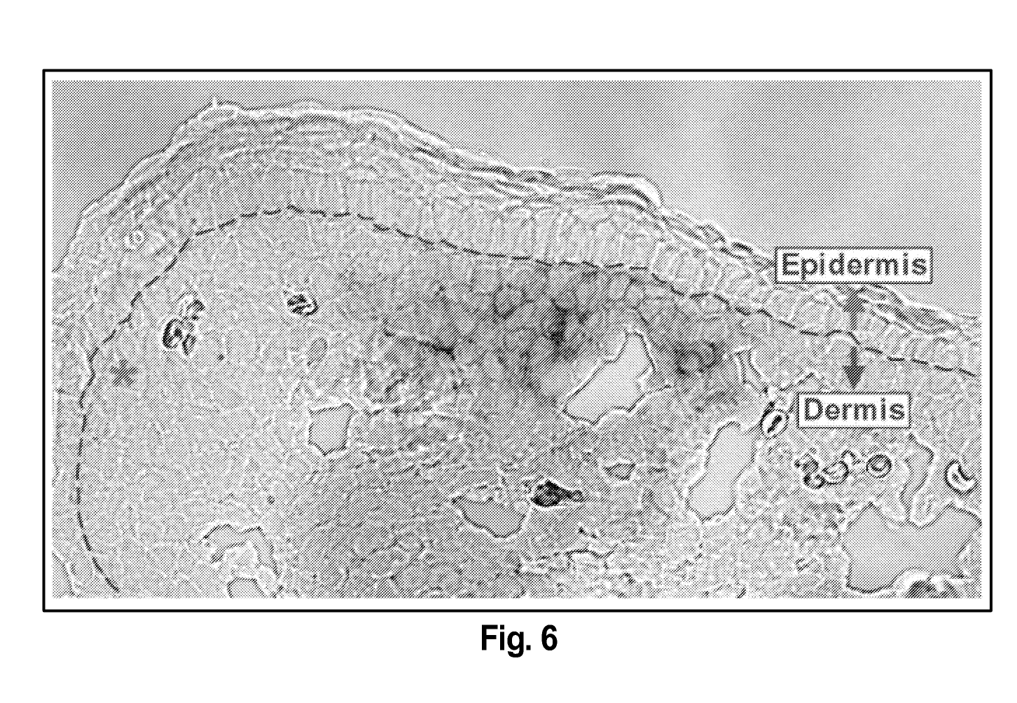

Parametric multipoint linkage analysis was performed with Allegro (Gudbjartsson, D. F., Jonasson, K., Frigge, M. L. & Kong, A. Allegro, a new computer program for multipoint linkage analysis. Nat Genet. 25, 12-3 (2000)) using a recessive model and complete penetrance. Analysis of three of the anonychia families revealed a single region with a LOD score of 4 on chromosome 20p13 (FIG. 1C). By combining the SNP data with the original microsatellite data in the consanguineous Pakistani and Finnish families, the minimal region of homozygosity was mapped to between 161,423 bp (SNP rs1342841) and 1,453,576 bp (D20S906) on chromosome 20p13, a region of 1292 Kbp (FIG. 1D). Three additional consanguineous Pakistani families (P2-P4) with non-syndromic hyponychia containing 28, 7 and 6 affected individuals respectively were also found to map to the same region on chromosome 20p13 (FIG. 3). A recombination in the P2 family reduced the region to 850 Kbp between microsatellite markers D20S117 and D20S906. Additional microsatellite markers in the region (Table 2 for primer sequences) were genotyped in family P2 (FIG. 2A and FIG. 2B) and informative further recombination events narrowed the minimal region harboring the disease gene to a small region of approximately 300 Kbp between 796,140 bp and 1,111,898 bp on chromosome 20p13 (FIG. 2A and FIG. 2B and FIG. 1D).

TABLE 2

Primers for additional microsatellite markers between D20S117 and
D20S906 and for mutation analysis of genes in the candidate region.

| | | | | | | |
|---|---|---|---|---|---|---|
| | | | | | Re- | For- |
| | | | | Ampli- | verse | ward |
| | | | | fied | Primer | primer |
| | | | | Size | SEQ ID | SEQ ID |
| Primer Name | Position (bp) | Forward (5′ to 3′) | Reverse (5′ to 3′) | (bp) | NO. | NO |

| | | | | | | |
|---|---|---|---|---|---|---|
| SCRT2-C20orf54 | 619,381-619,576 | CCCTGTGGTGTTAGATTGGA | CGTGGCTACATGGTGTATTG | 196 | 32 | 89 |
| MS1 | | | | | | |
| SCRT2-C20orf54 | 671,727-671,877 | CACCCCAGCAGGCATTGATT | GAGTGAGGACCTTCTAGGAA | 151 | 33 | 90 |
| MS2 | | | | | | |
| C20orf54-55 MS | 763,775-763,969 | CTCACACAGCCTTCATGAAG | GGACAGGGCAGTGGTTTCAT | 195 | 34 | 91 |
| C20orf55- | 778,667-778,822 | CTTCTGGTACTTTCCTCCAT | GGCAACAGAGCAAGATTCTG | 156 | 35 | 92 |
| ANGPT4 MS1 | | | | | | |
| C20orf55- | 795,958-796,140 | GACCTACCACTGATCTTGTT | ACCTTGGGCAACATAGCAAG | 182 | 36 | 93 |
| ANGPT4 MS2 | | | | | | |
| C20orf46 Ms | 1,111,898-1,112,047 | GCACTGAGGCTCTTTGAGTT | CCGGAGTTTATTCTCCAGTG | 150 | 37 | 94 |
| SNPH MS | 1,218,111-1,216,235 | TCTGGACCATGCCTGTCTTT | TTACAGGTGTGAGCCACCAT | 125 | 38 | 95 |
| FKBP1A-NSFL1C | 1,337,189-1,337,308 | GCCACTTCTTTGAGTCTTCA | ATTGCACCACTGCACTCCAA | 120 | 39 | 96 |
| MS | | | | | | |
| ANGPT4 ex1 | SEQ ID NO: 40 | CAGCCGTGGTATTCAGAGCAAGTA | GGATGGACACTCCACCTGCTGATT | 559 | 63 | |
| ANGPT4 ex2 | SEQ ID NO: 41 | GGATAGTCCAGGCAAGACGTAATG | ACCCTACTCAGGGTCAGAGATCAA | 418 | 64 | |
| ANGPT4 ex3 | SEQ ID NO: 42 | CTGGGAGAGTGGAAATGGGTAAGT | CCCTAGGTCCTAAACTTGACTCCA | 308 | 65 | |
| ANGPT4 ex4 | SEQ ID NO: 43 | CACAGGACGTTCCACCACACTTGA | GCCTGGATTGGGATTGTTGTTGAC | 561 | 66 | |
| ANGPT4 ex5 | SEQ ID NO: 44 | CAACCCAGAACCTGGCACAAAGCA | CCCCTACACCTCTGGTATTTCAGA | 340 | 67 | |
| ANGPT4 ex6 | SEQ ID NO: 45 | GGTCTGTCTGCTTAGCCACATTTG | GCAGATGTGCACTGTCAGCTTTAG | 332 | 68 | |
| ANGPT4 ex7 | SEQ ID NO: 46 | GCCTTAGTCTTTTCCCTCTAGCAG | GACCGTTGGAGCAGACTCTGTAGA | 458 | 69 | |
| ANGPT4 ex8 | SEQ ID NO: 47 | GACAAAGCCACTGGGGAAGTTCTT | TGGAGGGGACTTCAAGGACTCAAT | 414 | 70 | |
| ANGPT4 ex9 | SEQ ID NO: 48 | GCAACAGCCCCGATTAGTCTTTGT | GGCAGCTTCCGATGTGCAAATACA | 642 | 71 | |
| RSPO4 ex1 | SEQ ID NO: 49 | CCAACGCCCTCACTAGACCT | GTTGAGACTCGTCTGGAGGAGCGA | 381 | 72 | |
| RSPO4 ex2 | SEQ ID NO: 50 | CCATCTCAGCTGCTCGCATATATG | CTGGGCTTAGACATGCACCTACTT | 385 | 73 | |
| RSPO4 ex3 | SEQ ID NO: 51 | CACTGAGTCCTGACCCAAATGCTA | CCCTCACCATATGGCATTCTACTG | 383 | 74 | |
| RSPO4 ex4 | SEQ ID NO: 52 | TCAAACCCTGCCCTTGGATCTGAA | CCTTTCAGGCAGTCTCATAGATAC | 378 | 75 | |
| RSPO4 ex5 | SEQ ID NO: 53 | GGCACCCTTGTCTTTCAGGACTGA | CGAGGACTAGGACCAGAGAGT | 234 | 76 | |
| PSMF1 ex1 | SEQ ID NO: 54 | CTGCAGCCACCAGCCAAGTTCTTT | CCCCTGATCCATCCAGCACTTTCT | 192 | 77 | |
| PSMF1 ex2 | SEQ ID NO: 55 | CGTCTCCATTTTGGTCTCAGGTGT | GGTACTCTGAGTTTGGGCAGAAGA | 335 | 78 | |
| PSMF1 ex3 | SEQ ID NO: 56 | CATCTGTGAAGTGAGGTGGGTAAG | GAAGCTCTGGATTCGTACCGTTAA | 347 | 79 | |
| PSMF1 ex4 | SEQ ID NO: 57 | GTCTCCAAGCCTTAGGAAGGTATT | GGACACATCCACCCTATTCCTCAT | 329 | 80 | |
| PSMF1 ex5 | SEQ ID NO: 58 | GGTGAGGACAGAGGAGTAGCCAAT | GGATAGCTGGCTGGAATCCCTCTA | 557 | 81 | |
| PSMF1 ex6 | SEQ ID NO: 59 | CCCTTGTGCTATGGTCTCATGCAA | GGCTGAAAAGCCACAAAAGCAAGT | 229 | 82 | |
| PSMF1 ex7 | SEQ ID NO: 60 | GCCTTTTCTCCAAGGGCAGTCCTT | ACCTTCATTGCTGCCACACTGAAC | 293 | 83 | |
| PSMF1 ex8 | SEQ ID NO: 61 | CCTCACACCGCCACATCATGTTGA | GTCTGCAAACACATGAGCAGAATC | 290 | 84 | |
| PSMF1 ex8-2 | | | AGCCTGGTGCTCTATCGTGCTCTT | 1386 | 85 | |
| C20orf46 ex2 | SEQ ID NO: 62 | CCTGACTCACCTTCATGTGCTTAG | TGGACCTTGTAGCACTGGAGCTAA | 885 | 86 | |

The minimal region contained three genes; ANGPT4, RSPO4, PSMF1 and an open-reading frame, C20orf46 (FIG. 2B). Interestingly, RSPO4 encodes Rspondin 4, a member of the R-spondin family of secreted proteins that appear to act as a new class of frizzled ligands and activate the Wnt/beta-catenin pathway, leading to TCF-dependent target gene transactivation (Kamata, T. et al. R-spondin, a novel gene with thrombospondin type 1 domain, was expressed in the dorsal neural tube and affected in Wnts mutants. Biochim Biophys Acta 1676, 51-62 (2004); Nam, J. S., Turcotte, T. J., Smith, P. F., Choi, S. & Yoon, J. K. Mouse cristin/Rspondin family proteins are novel ligands for the Frizzled 8 and LRP6 receptors and activate beta-catenin-dependent gene expression. J Biol Chem 281, 13247-57 (2006); Kim, K. A. et al. R-Spondin proteins: a novel link to beta-catenin activation. Cell Cycle 5, 23-6 (2006)). Hence, as a potential developmental signalling molecule, RSPO4 was the best candidate for playing a role in nail development and was analysed first. All five exons were amplified by PCR and sequenced (Table 2).

Homozygous mutations were identified in RSPO4 in all four Pakistani families as well as the Finnish family while compound heterozygous mutations were identified in the three families from the UK (Table 1). Examples of sequence traces of some of the mutations are shown in FIG. 2C. Family PI has a 16 bp deletion in exon 2 that is predicted to cause a frameshift and premature downstream termination codon. Family P2 has a homozygous 26 bp deletion, which includes the initiating methionine codon in exon 1 and is predicted to lead to expression of a protein lacking the first 16 amino acid residues. Family P3 has a homozygous missense mutation of a cysteine to a tyrosine in exon 3 (C118Y). Family P4 has a homozygous 5′ donor splice site mutation and the Finnish family has a missense mutation of a glutamine to arginine residue in exon 2 (Q65R). In addition, affected individuals from the non-consanguineous Irish family and two additional families from England were found to be compound heterozygotes for a combination of splice site mutations and missense mutations involving cysteine residues in exon 3. Several of the mutations identified in this group of 8 families are recurrent.

TABLE 1
Summary of mutations detected in
all anonychia families analysed.

| Family | Mutation |
|---|---|
| P1 | 95 − 110del16 |
| P2 | −9 −+ 17del26 |
| P3 | 353G > A (C118Y) |
| P4 | IVS1 + 1G > A |
| F | 194A > G (Q65R) |
| I | 319T > C (C107R) |
| | IVS1 + 1G > A |
| E1 | 284G > T (C95F) |
| | IVS1 − 1G > A |
| E2 | 284G > T (C95F) |
| | 319T > C (C107R) |

RSPO4 mRNA and protein sequences according to NM_001029871 with nucleotide numbering starting from the first ATG codon. Amino acid substitutions are shown in brackets with reference to protein sequence NP_001025042.

The R-spondin family of proteins, of which there are four members in both the human and mouse genomes, share a common genomic and protein domain organization, and are conserved through vertebrate evolution. Each consists of five coding exons that are predicted to encode an N-terminal signal peptide (exon 1), followed by two furin-type cysteine-rich domains (exons 2 and 3), a thrombospondin-type domain (exon 4) and ending in a C-terminal basic region that scores highly as a putative nuclear localization signal (exon 5) (FIG. 2D). The furin-like repeats encoded by exons 2 and 3 are believed to be required for activation and stabilization of beta-catenin(Kazanskaya, O. et al. R-Spondin2 is a secreted activator of Wnt/beta-catenin signaling and is required for Xenopus myogenesis. Dev Cell 7, 525-34 (2004)). Therefore, mutations that disrupt the furin-like domains may affect signalling through beta-catenin. In this regard, it is noteworthy that all three cysteine mutations identified reside in exon 3. The residues affected in each of the missense mutations identified here are highly conserved across all four human R-spondin paralogues, as well as in all four mouse R-spondin paralogues and in a predicted protein from the invertebrate sea urchin, S. purpuratus 13,14. The conservation of the cysteine mutated at residue 95, 107, 118 (C95F, C107R and C118Y) is shown in FIG. 2E.

The splice site mutations identified here all alter the highly conserved GT or AG consensus sequences found at the 5′ and 3′ ends of introns, respectively, and hence would be predicted to lead to inappropriate exon skipping or intron inclusion in the mature mRNA transcript. The 26 bp deletion identified in family P2 encompasses the first ATG codon, however, it is predicted that protein translation may commence from the second ATG codon and result in a protein lacking the first 16 residues encoding the putative signal peptide. Finally, the 16 bp deletion in family P1 results in a frameshift and downstream premature termination codon. A truncated protein, if synthesized, would result from missense coding after residue 32 and is predicted to a give rise to a putative 220-residue truncated protein that would lack any features of an R-spondin protein.

To visualise the expression of RSPO4 in early nail development, whole mount in situ hybridization was performed using a 598 bp cDNA murine RSPO4 probe. In situ hybridization was performed on el 5.5 embryos as per detailed published protocols (Wilkinson, D. G. In situ hybridization: a practical approach. Oxford University Press (1992)). The expression pattern of RSPO4 is very specific and was only detectable in the mesenchyme from which the nails are derived, with some expression in the whisker pad (FIG. 2F). Additionally, it was also noted that RSPO4 expression was absent in mouse tissues at embryonic day 14.5 and appeared for the first time at day 15.5, initially in the forelimbs and later in the hindlimbs. This expression data further supports a highly specific and essential role for RSPO4 in nail development.

The Wnt signalling pathway (FIG. 7) plays a crucial role in numerous processes in animal development and so it is not surprising that a member of the R-spondin family of proteins implicated in the Wnt pathway is essential for nail development. Though other members of the R-spondin family have recently been implicated in vertebrate development (Kamata, T. et al. R-spondin, a novel gene with thrombospondin type 1 domain, was expressed in the dorsal neural tube and affected in Wnts mutants. Biochim Biophys Acta 1676, 51-62 (2004); Nam, J. S., Turcotte, T. J., Smith, P. F., Choi, S. & Yoon, J. K. Mouse cristin/Rspondin family proteins are novel ligands for the Frizzled 8 and LRP6 receptors and activate beta-catenin-dependent gene expression. J Biol Chem 281, 13247-57 (2006); Kazanskaya, O. et al. R-Spondin2 is a secreted activator of Wnt/beta-catenin signaling and is required for Xenopus myogenesis. Dev Cell 7, 525-34 (2004)), this is the first evidence of a role for R-spondins in human disease. R-spondin 4 is almost certainly involved in the later phases of embryonic nail development and/or maintenance during adult life, since the affected individuals have no underlying bone deformities in the distal phalanges plus the nail bed as well as the nail folds that delineate the edges of the nail bed all appear to be fully formed.

### Example 2

### Mutations in R-spondin 4 (RSPO4) Underlie Inherited Anonychia

Recently, mutations in the RSPO4 gene were reported to underlie inherited anonychia/hyponychia (see Example 1). Here, five consanguineous Pakistani families with recessive inheritance of a combination of anonychia and hyponychia were studied. Homozygous mutations were identified in the RSPO4 gene in all five families. Three families had a splice site mutation at the exon 2-intron 2 boundary. One family had a 26 bp deletion encompassing the start codon, and the final family had a missense mutation changing the initiating methionine to isoleucine. Using in situ hybridization, Rspo4 was shown to be exclusively expressed in the mesenchyme underlying the digit tip epithelium in the mouse at embryonic day 14.5 (e14.5). These findings expand the understanding of the role of RSPO4 in nail development and disease.

Case description: Congenital absence of the nails in humans is referred to as anonychia/hyponychia congenita (OMIM 206800), a rare autosomal recessive condition in which the only phenotype is the absence or severe hypoplasia of all fingernails and toenails. Using homozygosity mapping, a region of linkage on chromosome 20p13 has been identified and a spectrum of mutations in the R-spondin 4 (RSPO4) gene in several affected families from India, Pakistan, Finland and the UK (Example 1) has been demonstrated. An independent study similarly reported mutations in RSPO4 in a family with hyponychia from Germany (Bergmann et al, 2006 Am J Hum Genet. 79, 1105-1109).

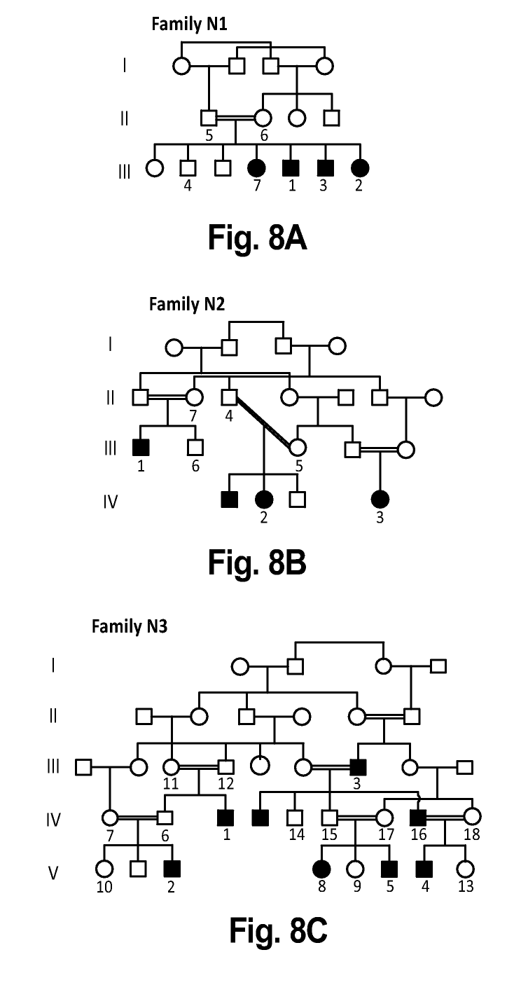

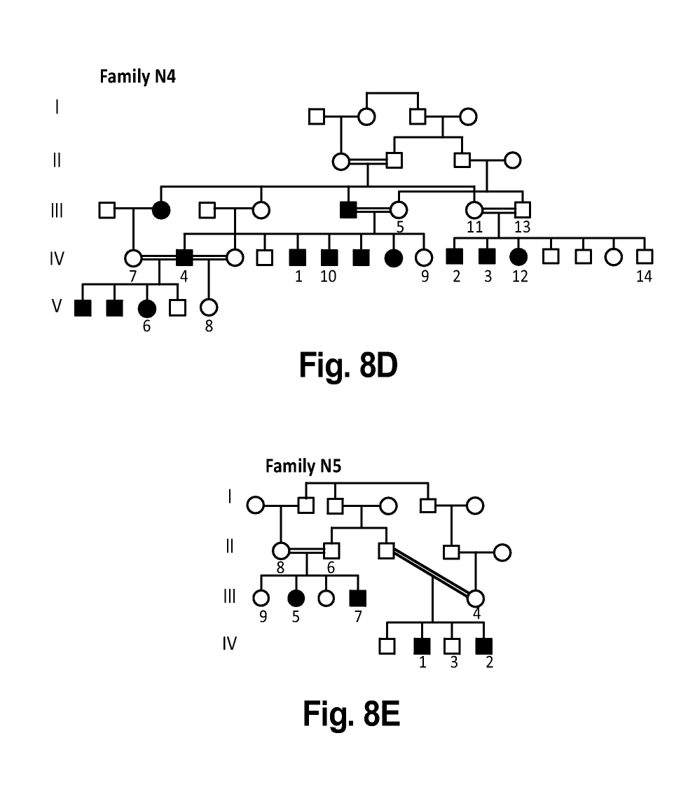

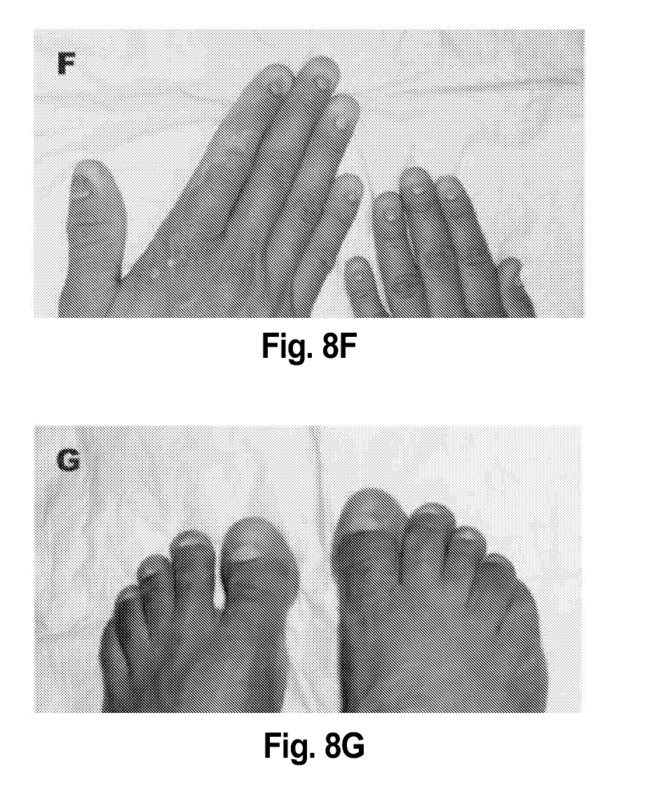

To further investigate the molecular basis of anonychia, five consanguineous families from Pakistan (N1-N5) that show recessive inheritance of a combination of anonychia and hyponychia (FIG. 8A-E) were studied. The five families come from different geographic regions of Pakistan. All patients exhibited either complete absence of the nail plate and matrix, with only the nail bed present, or hyponychia, with some remnants of rudimentary, fragile nail plates (FIG. 8F-G). No evidence for associated anomalies of ectodermal appendages, including hair, teeth, and sweat glands was noted in any of the affected individuals.

Mutation Identification

DNA was obtained from 55 members of the five families, including 25 affected and 30 unaffected individuals. The medical ethical committee of Columbia University approved all described studies. The study was conducted according to Declaration of Helsinki Principles and participants gave their written informed consent. Genomic DNA was isolated from peripheral blood collected in EDTA-containing tubes using the PUREGENE DNA isolation kit (Gentra System, USA). All samples were collected after informed consent had been obtained and in accordance with the local institutional review board.

To confirm that each family was linked to chromosome 20, genotyping was first performed using the markers D20S117, D20S199 and D20S906, which are closely mapped to the RSPO4 gene. All anonychia families were found to be linked for each of the three markers.

To screen for a mutation in the human RSPO4 gene, exons 1-5 as well as flanking splice junctions were PCR amplified from genomic DNA. The primers used for the PCR were previously described (see Example 1). After purification in Performa DTR gel filtration cartridges (Edge Biosystems, Gaithersburg, Md.), PCR fragments were directly sequenced by an ABI PRISM 310 automated sequencer (Applied Biosystems, Foster City, Calif.) with a Big Dye terminator cycle sequencing kit (Applied Biosystems, Foster City, Calif.) and the primers. Homozygous mutations were identified in RSPO4 in all five Pakistani families. Families N1, N2 and N4 have a novel splice site mutation at the exon 2-intron 2 boundary (IVS-1G>A), predicted to result in aberrant splicing of RSPO4 (FIG. 9A). Family N3 has a 26 bp deletion, which includes the start codon in exon 1 and is predicted to lead to expression of a protein lacking the first 16 amino acid residues (FIG. 9B). Family N5 has a missense mutation changing the initiating methionine of RSPO4 to isoleucine (M1I) (FIG. 9C). Each of these mutations is predicted to severely impair the synthesis of a functional RSPO4 protein. Two of these mutations are novel, while the third (N3: −9-+17del26) was previously identified in our earlier studies (see Example 1, family P2). No correlation has thus far been observed between the various mutations detected and specific phenotypic alterations in the small number of patients studied here and in previous studies. FIG. 9D and Table 3 summarizes all previously reported RSPO4 mutations from our group and others, as well as those identified in this study.

TABLE 3
Summary of RSPO4 mutations in various families.

| Family | Mode of mutation | Mutation |
|---|---|---|
| P1 | Homozygous mutant | 95 − 110del16 |
| P2 | Homozygous mutant | −9 −+ 17del26 |
| P3 | Homozygous mutant | 353G > A (C118Y) |
| P4 | Homozygous mutant | IVS1 + 1G > A |
| F | Homozygous mutant | 194A > G (Q65R) |
| I | compound heterozygous | 319T > C (C107R) |
| | | IVS1 + 1G > A |
| E1 | compound heterozygous | 284G > T (C95F) |
| | | IVS1 − 1G > A |
| E2 | compound heterozygous | 284G > T (C95F) |
| | | 319T > C (C107R) |
| N1 | Homozygous mutant | IVS2 − 1G > A |
| N2 | Homozygous mutant | IVS2 − 1G > A |
| N3 | Homozygous mutant | −9 −+ 17del26 |
| N4 | Homozygous mutant | IVS2 − 1G > A |
| N5 | Homozygous mutant | 3G > A (M1I) |

Recently, a detailed analysis of the expression patterns of the four Rspo family members during mouse embryogenesis was reported (Nam et al, 2006 Gene Expr Patterns 7, 306-312). Interestingly, Rspo4 expression was detected from e7-e17 by RT-PCR on cDNA derived from mouse embryos. Whole mount in situ hybridization during embryogenesis revealed Rspo4 expression in the groove of the neural fold at e8.5, in the forebrain at e9.5 and in the developing heart and limbs from e9.5-e10.5. From e15.5-e17.5, Rspo4 expression was observed in a number of tissues, with the highest level of expression in the developing tooth and various elements of the skeleton (Nam et al, 2006). As inferred from the anonychia phenotype, this broad pattern of expression suggests that in all body sites except the digit tip, the function of Rspo4 may be compensated for by the presence of another family member.

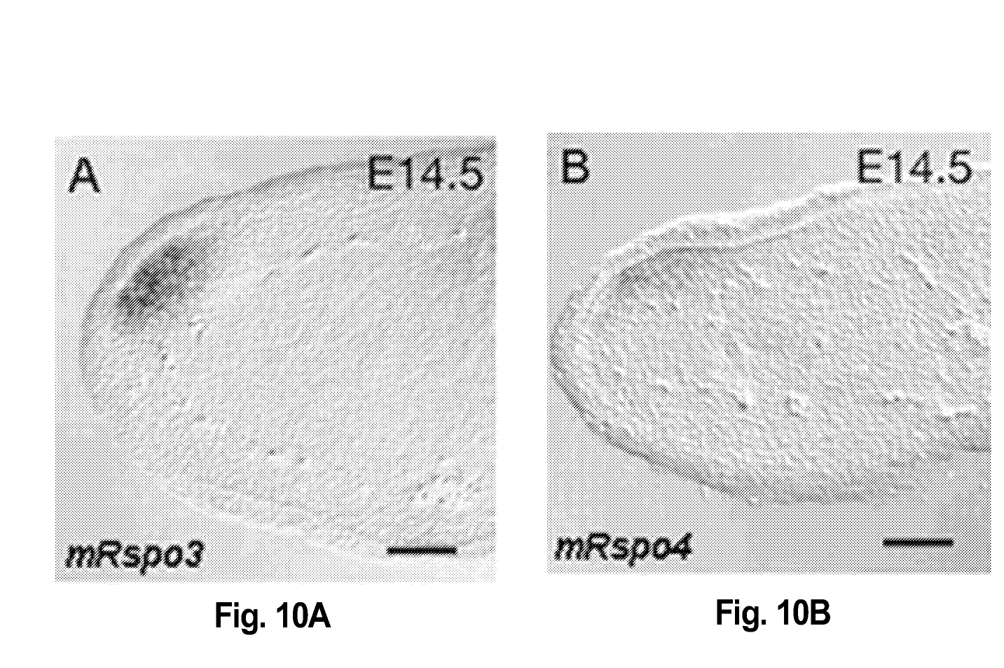

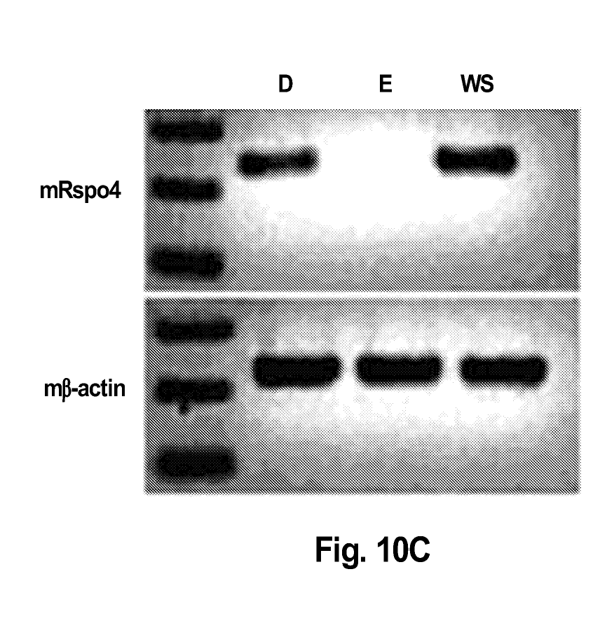

Rspo4 expression has previously been shown in the tip of the digits, arising between e14.5-e15.5 (see Example 1; Nam et al, (2006) Gene Expr Patterns 7, 306-312). In order to localize the expression of Rspo4 and Rspo3 to a particular compartment in early nail development, whole mount in situ hybridization was performed on e14.5 mouse embryos. Digoxigenin (DIG)-labeled antisense (AS) riboprobes specific to the mouse Rspo3 and Rspo4 genes were synthesized and in situ hybridization was performed on the limbs of e14.5 embryos as described (Nam et al, 2006 Gene Expr Patterns 7, 306-312). The stained embryos were post-fixed in 4% paraformaldehyde in PBS, and cryosectioned after embedding in Tissue-Tek® OCT compound (Fisher Scientific, Hampton, N.H.). The images of sections were obtained using an HRC Axiocam fitted onto an Axioskop2 plus microscope (Carl Zeiss, Thornwood, N.Y.). Rspo4 was shown to be exclusively expressed in the mesenchyme underlying the digit tip epithelium (FIG. 10B). Moreover, in comparison to Rspo3, which is expressed more intensely and in a broader region of the digit tip (FIG. 10A), Rspo4 expression is weaker and more restricted.

To further extend these findings, the expression of Rspo4 by RT-PCR was examined in e14.5 mouse dermis and epidermis from dorsal skin. Dissected skin was enzymatically digested, allowing for a separation of the epidermis from the dermis. RNA was subsequently extracted from each dissected tissue using an RNeasy Mini Kit (Qiagen, Valenica, Calif.). RNA was also extracted from adult mouse dorsal whole skin. Reverse transcription was carried out using Oligo (dT) primer and SuperScript™ III (Invitrogen, Carlsbad, Calif.) according to the manufacturer's instructions.

The primers mRspo4 F: 5′-CAGCAGAGGCTCTTCCTCTTCATC-3′ and mRspo4 R: 5′-GAGCCACAGGTCTTCCCATTGTGT-3′ were used to amplify a 326 bp product. Consistent with the in situ hybridization results, the expression of Rspo4 was restricted to the dermis and was not present in the epidermis (FIG. 10C).

We conclude that although Rspo3 is expressed at the same place and time as Rspo4 in the mouse digit tip, RSPO3 is apparently not able to compensate for RSPO4 in this region in humans, since the phenotype arises in the absence of RSPO4 despite the presence of RSPO3. Given that RSPO3 is located on human chromosome 6, it is not a candidate in human anonychia. In mice, Rspo3 knockouts die at e10 due to abnormal placental development (Aoki et al, 2006 Dev Biol. 301, 218-226). To date, we have found no evidence for locus heterogeneity, and all anonychia families studied thus far are both linked to and have mutations in RSPO4 on chromosome 20.

What is claimed is:

1. A method for treating a nail, hoof, or claw keratin-related abnormality in a subject, the method comprising:
a) administering to the subject an effective amount of a composition comprising a R-spondin 4 modulating compound,
thereby treating keratin-related abnormality in the subject.

2. The method of claim 1, wherein the abnormality is characterized by weakening of the nail, hoof, or claw.

3. The method of claim 1, wherein the abnormality is characterized by slow or absent growth or repair of the nail, hoof, or claw.

4. The method of claim 1, wherein the abnormality is characterized by hyperplasia of the nail, hoof, or claw.

5. The method of claim 1, wherein the abnormality is an inherited abnormality.

6. The method of claim 5, wherein the inherited abnormality is selected from the group consisting of anonychia congenita, hyponychia congenita, Cooks syndrome, nail patella syndrome, ectodermal dysplasias, and epidermolysis bullosa.

7. The method of claim 1, wherein the abnormality is caused by an infection of the nail, hoof, or claw.

8. The method of claim 7, wherein the infection is caused by a bacterium, a fungus, a yeast, a mold, a virus, or any combination thereof.

9. A method for strengthening, repairing, or stimulating growth of a nail, hoof, or claw in a subject, the method comprising:
a) administering to the subject an effective amount of a composition comprising a R-spondin 4 modulating compound, wherein the compound increases the activity or the expression of R-spondin 4.

10. A method for inhibiting the growth of, or weakening, a nail, hoof, or claw in a subject, the method comprising:
a) administering to the subject an effective amount of a composition comprising a R-spondin 4 modulating compound, wherein the compound decreases the activity or the expression of R-spondin 4.

11. The method of claim 1 or 10, wherein the compound comprises an antibody directed to R-spondin 4 comprising SEQ ID NO: 1, or a fragment thereof.

12. The method of claim 1 or 10, wherein the compound comprises a R-spondin 4 antisense RNA or antisense DNA; a R-spondin 4 siRNA; or a combination thereof.

13. The method of claim 12, wherein the compound decreases expression of R-spondin 4 via RNA interference.

14. The method of claim 12, wherein R-spondin 4 comprises a polynucleotide molecule comprising SEQ ID NO: 2, 27, 28, 29, 30, or 31.

15. The method of claim 1, 9 or 10, wherein the compound comprises a R-spondin 4 polypeptide molecule comprising at least 10 amino acids of SEQ ID NO: 1, or a fragment, variant, or peptidomimetic thereof.

16. The method of claim 1, 9 or 10, wherein the compound comprises a R-spondin 4 peptide comprising at least 5 amino acids of SEQ ID NO: 22, or a fragment, variant, or peptidomimetic thereof.

17. The method of claim 1, 9 or 10, wherein the compound comprises a R-spondin 4 peptide comprising at least 5 amino acids of SEQ ID NO: 23, or a fragment, variant, or peptidomimetic thereof.

18. The method of claim 1, 9 or 10, wherein the compound comprises a R-spondin 4 peptide comprising at least 5 amino acids of SEQ ID NO: 24, or a fragment, variant, or peptidomimetic thereof.

19. The method of claim 1, 9 or 10, wherein the compound comprises a R-spondin 4 peptide comprising at least 5 amino acids of SEQ ID NO: 25, or a fragment, variant, or peptidomimetic thereof.

20. The method of claim 1, 9 or 10, wherein the compound comprises a R-spondin 4 peptide comprising at least 5 amino acids of SEQ ID NO: 26, or a fragment, variant, or peptidomimetic thereof.

21. The method of claim 1, 9 or 10, wherein the compound comprises a R-spondin 4 peptide having at least 46%, 48%, 50%, 55%, 60%, 70%, 75%, 80%, 90%, 95%, or 99% identity to SEQ ID NO: 1.

22. The method of claim 1, 9 or 10, wherein the subject is a human, a cat, a dog, a horse, a cow, a sheep, a goat, a pig, a chicken, an avian, a domestic pet, or a mammal reared for agricultural uses.

23. The method of claim 1, 9 or 10, wherein the composition is administered to, or in the vicinity of, one of more nails, hooves, or claws.

24. The method of claim 1, 9 or 10, wherein the composition is administered topically.

25. The method of claim 1, 9 or 10, wherein the composition is formulated as a cream or lotion, an oil, or as a paint or lacquer.

26. The method of claim 1, 9 or 10, wherein the composition comprises one or more carriers, excipients, solvents or bases.

27. A method for identifying a compound that modulates R-spondin 4 activity, the method comprising:
a) expressing R-spondin 4 in a cell;
b) contacting the cell with a ligand source for an effective period of time;
c) measuring a secondary messenger response;
d) isolating the ligand from the ligand source; and
e) identifying the structure of the ligand that binds R-spondin 4,
thereby identifying which compound would modulate the activity of R-spondin 4.

28. The method of claim 27, further comprising:
f) obtaining or synthesizing the compound determined to bind to R-spondin 4 or to be a potential modulator of R-spondin 4 activity;
g) contacting R-spondin 4 protein with the compound under a condition suitable for binding; and
h) determining whether the compound modulates R-spondin 4 activity using a diagnostic assay.

29. The method of claim 27, wherein the compound is a R-spondin δ agonist or a R-spondin 4 antagonist.

30. The method of claim 29, wherein the antagonist decreases R-spondin 4 expression or R-spondin 4 activity by at least 10%, 20%, 30%, 40%, 50%, 60%, 70%, 75%, 80%, 90%, 95%, 99%, or 100%.

31. The method of claim 29, wherein the agonist increases R-spondin 4 expression or R-spondin 4 activity by at least 10%, 20%, 30%, 40%, 50%, 60%, 70%, 75%, 80%, 90%, 95%, 99%, or 100%.

32. The method of claim 27, wherein the compound comprises an antibody directed to R-spondin 4 or a fragment thereof, a R-spondin 4 antisense RNA or antisense DNA; a R-spondin 4 siRNA; or a R-spondin 4 peptide comprising at least 10 amino acids of SEQ ID NO: 1, or a fragment or variant thereof.

33. The method of claim 27, wherein the cell is a bacterium, a yeast, an insect cell, or a mammalian cell.

34. The method of claim 27, wherein the ligand source is a compound library.

35. The method of claim 27, wherein measuring comprises detecting an increase or decease in a secondary messenger concentration.

36. The method of claim 27, wherein the assay determines the concentration of the secondary messenger within the cell.

37. The method of claim 35 or 36, wherein the secondary messenger comprises Tcf1, Lef1, phosphorylated Dsh, Axin, β-catenin, or a combination thereof.

38. The method of claim 28, wherein contacting comprises administering the compound to a mammal in vivo or a cell in vitro.

39. The method of claim 38, wherein the mammal is a mouse.

40. An isolated mutant human R-spondin 4 polypeptide comprising a C>Y mutation at amino acid position 118 of SEQ ID NO: 1, comprising the amino acid sequence of SEQ ID NO: 6.

41. An isolated mutant human R-spondin 4 polypeptide comprising a M>I mutation at amino acid position 1 of SEQ ID NO: 1, comprising the amino acid sequence of SEQ ID NO: 14.

42. An isolated mutant human R-spondin 4 polypeptide encoded by a nucleic acid comprising the sequence of SEQ ID NO: 11.

43. An isolated mutant human R-spondin 4 polynucleotide comprising the nucleic acid sequence of SEQ ID NO: 10, 11, or 15.

44. A pharmaceutical, veterinary, or cosmetic composition comprising a R-spondin 4 polypeptide molecule having at least 46%, 48%, 50%, 55%, 60%, 70%, 75%, 80%, 90%, 95%, or 99% identity to SEQ ID NO: 1, or a variant, fragment, or peptidomimetic thereof.

45. The composition of claim 44, wherein R-spondin 4 comprises at least 5 amino acids of an amino acid sequence comprising SEQ ID NO: 22, 23, 24, 25, or 26.

46. The composition of claim 44, wherein R-spondin comprises at least 10 amino acids of the amino acid sequence comprising SEQ ID NO: 1.

47. The composition of claim 44, wherein R-spondin 4 comprises R-spondin 4 antisense RNA or antisense DNA; a R-spondin 4 siRNA; or a combination thereof.

48. The composition of claim 47, wherein R-spondin 4 comprises a polynucleotide molecule comprising SEQ ID NO: 2, 27, 28, 29, 30, or 31.

49. The composition of claim 44, wherein the composition is formulated for administration to, or in the vicinity of, one of more nails, hooves or claws.

50. The composition of claim 44, wherein the composition is formulated for topical administration.

51. The composition of claim 44, wherein the composition is formulated as a cream or lotion, an oil, or a paint or lacquer.

52. The composition of claim 44, further comprising one or more carriers, excipients, solvents or bases.

53. The composition of claim 44, wherein the R-spondin is present in a therapeutically or cosmetically effective amount.

54. A method for diagnosing anonychia congenita in a subject, the method comprising testing the subject for a mutation in the R-spondin 4 gene, wherein a DNA sample is obtained from the subject.

55. The method of claim 54, wherein the subject is a human.

56. The method of claim 54, wherein the mutation comprises a nucleic acid sequence comprising SEQ ID NO: 11, wherein the first 26 nucleic acid residues from SEQ ID NO: 2 are deleted; a nucleic acid sequence comprising SEQ ID NO: 10, wherein a G>A mutation occurs at nucleic acid position +353 of SEQ ID NO: 2; a nucleic acid sequence comprising SEQ ID NO: 15, wherein an G>A mutation occurs at nucleic acid position +3 of SEQ ID NO: 2; or a combination thereof.

57. The method of claim 54, wherein the mutation comprises a nucleic acid encoding a polypeptide molecule comprising an amino acid sequence comprising SEQ ID NO: 6, wherein a C>Y mutation occurs at amino acid position 118 of SEQ ID NO: 1; a nucleic acid encoding a polypeptide molecule comprising an amino acid sequence comprising SEQ ID NO: 14, wherein a M>I mutation occurs at amino acid position 1 of SEQ ID NO: 1; or a combination thereof.

58. The method of claim 54, wherein the mutation comprises a nucleic acid comprising SEQ ID NO: 16, wherein a G>A mutation occurs at nucleic acid position 3077 of SEQ ID NO: 19; a nucleic acid comprising SEQ ID NO: 17, wherein a G>A mutation occurs at nucleic acid position 3711 of SEQ ID NO: 19; a nucleic acid comprising SEQ ID NO: 20, wherein a G>A mutation occurs at nucleic acid position 809 of SEQ ID NO: 19; or a combination thereof.

59. The method of claim 54, wherein the mutation comprises a G>A nucleic acid mutation at about nucleotide position 3853 of SEQ ID NO: 19, which lies at the intron 3-exon 3 boundary; a G>A nucleic acid mutation at about nucleotide position 4797 of SEQ ID NO: 19, which lies at the intron 3-exon 4 boundary; a G>A nucleic acid mutation at about nucleotide position 4984 of SEQ ID NO: 19, which lies at the intron 4-exon 4 boundary; a G>A nucleic acid mutation at about nucleotide position 6095 of SEQ ID NO: 19, which lies at the intron 4-exon 5 boundary; or a combination thereof.

60. The method of claim 54, wherein the mutation occurs in a nucleic acid sequence encoding a polypeptide molecule comprising SEQ ID NO: 22, 23, 24, 25, 26, or a combination thereof.

61. The method of claim 54, wherein the mutation attenuates the function of the R-spondin 4 protein or produces a truncated R-spondin protein.
